(19) United States
(12) Patent Application Publication
(10) Pub. No.: US 2024/0399094 A1
(43) Pub. Date: Dec. 5, 2024

(54) SLEEVE WITH MAGNET FOR MODULARITY AND HEADGEAR FOR THE SAME

(71) Applicant: ResMed Asia Pte. Ltd., Singapore (SG)

(72) Inventor: Beng Hai TAN, Singapore (SG)

(21) Appl. No.: 18/697,440

(22) PCT Filed: Oct. 28, 2022
(86) PCT No.: PCT/SG2022/050777

§ 371 Date: Mar. 29, 2024

(30) Foreign Application Priority Data
Oct. 29, 2021 (SG) 10202112048R

(51) Int. Cl. A61M 16/06

(52) U.S. Cl. CPC A61M 16/0683; A61M 16/0616; A61M 2210/0618; A61M 2210/0625

(57) ABSTRACT

A patient interface includes a plenum chamber pressurisable to a therapeutic pressure, a seal-forming structure constructed and arranged to form a seal with a region of the patient's face surrounding an entrance to the patient's airways, and a positioning and stabilising structure to provide a force to hold the seal-forming structure in a therapeutically effective position on the patient's head. The positioning and stabilising structure includes an inextensible element configured to overlay the patient's cheeks, a sleeve configured to cover at least a portion of the inextensible element, and headgear straps configured to provide at least a portion of the force. The patient interface is configured to allow the patient to breathe from ambient through their mouth in the absence of a flow of pressurised air through the plenum chamber inlet port, or the patient interface is configured to leave the patient's mouth uncovered.

### 1 CROSS-REFERENCE TO RELATED APPLICATIONS

This application claims the benefit of Singapore Provisional Application No. 10202112048R, filed Oct. 29, 2021, the entire contents of which is incorporated herein by reference in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063328 and WO 2006/130903 (describing amongst other things aspects of the ResMed Limited MIRAGE LIBERTY™ full-face mask); International Patent Application WO 2009/052560 (describing amongst other things aspects of the ResMed Limited SWIFT™ FX nasal pillows).

### 2.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm an applied generally to the face area in and about the patient interface is more comfortable than cold a.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034665; International Patent Application Publication No. WO 2000/078381; U.S. Pat. No. 6,581,594; US Patent Application Publication No. US 2009/0050156; US Patent Application Publication No. 2009/0044808.

Table of noise of prior masks (ISO 17510-22007, 10 cmH20 pressure at 1 m)

| | | A-weighted | A-weighted | |
|---|---|---|---|---|
| | | sound power | sound | |
| | Mask | level dB(A) | pressure dB(A) | Year |
| Mask name | type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed | nasal | 32 (3) | 24 (3) | 2002 |
| Mirage | | | | |
| ActivaTM | | | | |
| ResMed | nasal | 30 (3) | 22 (3) | 2008 |
| Mirage | | | | |
| MicroTM | | | | |
| ResMed | nasal | 29 (3) | 22 (3) | 2008 |
| MirageTM | | | | |
| SoftGel | | | | |
| ResMed | nasal | 26 (3) | 18 (3) | 2010 |
| MirageTM FX | | | | |
| ResMed | nasal | 37 | 29 | 2004 |
| Mirage | pillows | | | |
| SwiftTM (*) | | | | |
| ResMed | nasal | 28 (3) | 20 (3) | 2005 |
| Mirage | pillows | | | |
| SwiftTM II | | | | |
| ResMed | nasal | 25 (3) | 17 (3) | 2008 |
| Mirage | pillows | | | |
| SwiftTM LT | | | | |
| ResMed AirFit | nasal | 21 (3) | 13 (3) | 2014 |
| P10 | pillows | | | |

(* one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a sleeve for facilitating the connection between headgear of the positioning and stabilizing structure and the cushion.

Another aspect of one form of the present technology is a series of modular elements that may be interconnected in order to form different styles of patient interfaces.

In one form, a) at least one sleeve is included in the modular elements; b) the sleeve is constructed from a comfortable material; and/or c) the at least one sleeve is selectively used in order to use a cushion in either a tube-up or tube-down arrangement.

In one form, a) a cushion is included in the modular elements; b) the cushion includes at least one first opening and at least one second opening; and/or c) a plenum chamber of the cushion is configured to receive pressurized air through either the at least one first opening or the at least one second opening depending on a selected style.

In one form, there are at least two versions or styles of each modular element. The versions or styles may be interchangeably used with one another in order to form different modular assemblies.

Another aspect of one form of the present technology is a sleeve configured to be selectively used in a modular patient interface, the sleeve configured to at least partially contain a portion of the positioning and stabilizing structure, and the sleeve having at least two connection points for removably connecting to straps of the headgear.

In one form, a) the sleeve is configured to substantially or completely cover rigidizer arms; b) the sleeve includes a pair of tabs configured to receive the straps; c) the sleeve includes an inferior extension with a connector configured to detachably connect to the straps; and/or d) the connector is a magnet.

In one form, a) the sleeve is configured to partially cover conduits for conveying pressurized air to the patient; b) the sleeve includes a pair of tabs configured to receive the straps; c) the sleeve includes an inferior extension with a connector configured to detachably connect to the straps; and/or d) the connector is a magnet.

In one form, the sleeve is configured to connect to a cushion for use in a tube-down configuration where pressurized airflow is conveyed to a patient from in front of the patient's head.

In one form, the sleeve is configured to connect to a cushion for use in a tube-up configuration where pressurized airflow is conveyed to a patient through conduit headgear.

Another aspect of one form of the present technology is a conduit sleeve configured to connect to a positioning and stabilizing structure of a patient interface, the conduit sleeve comprising: a longitudinal extension forming a pathway that extends between a superior opening and an inferior opening, the pathway configured to receive a fluid conduit; an inferior extension positioned outside of the pathway and proximate to the inferior opening; and a connection member connected to the inferior extension.

In some forms, a) the inferior extension is more rigid than the pathway; b) the inferior extension is form with a rigid material (e.g., plastic); c) the inferior extension is rigidized using a stitching method (e.g., flat knitting); and/or d) the inferior extension is substantially inextensible.

In some forms, a) the connection member is a magnet; and/or b) the connection member and the connection member are oriented in opposite directions.

In some forms, a) material around the superior opening and/or the inferior opening is elastic and configured to allow the superior opening and/or the inferior opening to stretch and expand a width of the respective opening; and/or b) the material between the superior opening and the inferior opening is substantially inextensible.

In some forms: a) the inferior opening is configured to be positioned proximate to a cushion of the patient interface, in use; b) the superior opening is configured to be positioned at a location inferior to the patient's ear, in use; and/or c) the conduit sleeve is one of a pair of conduit sleeves, each conduit sleeve of the pair of conduit sleeves configured to removably connect to a fluid conduit of a conduit headgear.

Another aspect of one form of the present technology is a four-point arm sleeve configured to connect to a positioning and stabilizing structure of a patient interface, the conduit sleeve comprising:
a superior section; and
a pair of inferior sections, each inferior section of the pair of inferior sections including,
   a pathway having an inferior opening, the pathway configured to receive a rigid arm,
   an inferior extension connected proximate to the inferior opening, the inferior extension positioned outside of the pathway,
   a connection member connected to the inferior extension, and
   a tab disposed proximate to the superior section and configured to receive a headgear strap.

In some forms, a) the inferior extension is more rigid than the pathway; b) the inferior extension is form with a rigid material (e.g., plastic); c) the inferior extension is rigidized using a stitching method (e.g., flat knitting); and/or d) the inferior extension is substantially inextensible.

In some forms, a) the connection member is a magnet; and/or b) the connection member and the connection member are oriented in opposite directions.

In some forms, a) material around the inferior opening is elastic and configured to allow the inferior opening to stretch and expand a width of the respective opening; b) the remaining inferior section is substantially inextensible; and/or c) the superior portion is substantially inextensible.

In some forms: a) the inferior opening is configured to be positioned proximate to a cushion of the patient interface, in use; b) the tab is configured to be positioned at a location superior to the patient's ear, in use; and/or c) the superior section.

Another aspect of one form of the present technology is a two-point arm sleeve configured to connect to a positioning and stabilizing structure of a patient interface, the conduit sleeve comprising:
a superior section; and
a pair of inferior sections, each inferior section of the pair of inferior sections including,
   a pathway having an inferior opening, the pathway configured to receive a rigid arm,
   a connection member connected to the inferior extension, and
   a tab disposed proximate to the superior section and configured to receive a headgear strap.

In some forms, a) material around the inferior opening is elastic and configured to allow the inferior opening to stretch and expand a width of the respective opening; b) the remaining inferior section is substantially inextensible; and/or c) the superior portion is substantially inextensible.

In some forms: a) the inferior opening is configured to be positioned proximate to a cushion of the patient interface, in use; b) the tab is configured to be positioned at a location superior to the patient's ear, in use; and/or c) the superior section.

Another aspect of one form of the present technology is a cushion configured to seal against a portion of a patient's face around an entrance to the patient's airways. The cushion includes at least one first opening and at least one second opening. One of the at least one first opening and at least one second opening configured to receive a flow of pressurized air and the other of the at least one first opening and at least one second opening configured to receive a plug to limit ingress and/or egress of pressurized air from a plenum chamber of the cushion.

In some forms, a) a first plug is removably received in the at least one first opening and a second plug is removably received in the at least one second opening; b) the first plug is different than the second plug; c) the at least one first opening includes a pair of first openings and a pair of first plugs are removably received within the pair of first openings when the at least one second opening is configured to receive the flow of pressurized air; and/or d) the first plug is connected to an elongated member.

Another aspect of one form of the present technology is a method of assembling a modular system comprising providing a positioning and stabilizing structure, and connecting the positioning and stabilizing structure to either a first cushion or a second cushion.

Another aspect of one form of the present technology is a method of assembling a modular system including:
providing interface structures including a first cushion configured to seal against a patient's nares and mouth and a second cushion configured to seal against the patient's nares and leave the patient's mouth exposed, wherein the first cushion and the second cushion each include inlet ports;
providing positioning and stabilizing structures including conduit headgear configured to convey the flow of pressurized air and rigidizer arms, wherein the conduit headgear and the rigidizer arms are configured to removably connect to the inlet ports of the first cushion or the second cushion;
providing sleeves including a conduit sleeve removably connected to the conduit headgear and an arm sleeve removably connected to the rigidizer arms, wherein the conduit sleeve and the arm sleeve each include at least two connection points;
providing headgear including a two-point headgear and a four-point headgear, wherein each of the two-point headgear and the four-point headgear configured to connect to the at least two connection points on either the conduit sleeve or the arm sleeve;
selecting one interface structure, one positioning and stabilizing structure, one sleeve, and one headgear; and
assembling the selected interface structure, positioning and stabilizing structure, sleeve, and headgear.

In some forms, at least one of the selected interface structure, positioning and stabilizing structure, sleeve, and headgear may be replaced with a different version or style and assembled into a different structure.

In some forms, different styles or versions of the interface structure, positioning and stabilizing structure, sleeve, and headgear may be interchangeable.

Another aspect of one form of the present technology comprises a patient interface comprising:
a plenum chamber pressurisable to a therapeutic pressure of at least 4 cmH2O above ambient air pressure, said plenum chamber including at least one plenum chamber inlet port sized and structured to receive a flow of air at the therapeutic pressure for breathing by a patient,
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a positioning and stabilising structure to provide a force to hold the seal-forming structure in a therapeutically effective position on the patient's head, the positioning and stabilising structure including:
   an inextensible element configured to overlay the patient's cheeks,
   a sleeve configured to cover at least a portion of the inextensible element, and
   headgear straps configured to provide at least a portion of the force;
wherein the patient interface is configured to allow the patient to breathe from ambient through their mouth in the absence of a flow of pressurised air through the plenum chamber inlet port, or the patient interface is configured to leave the patient's mouth uncovered.

In one form, the inextensible element is a conduit headgear including a pair of conduits each configured to convey the flow of air at the therapeutic pressure to the plenum chamber; and the sleeve is a first conduit sleeve, the positioning and stabilising structure further includes a second conduit sleeve having the same structure as the first conduit sleeve, the second conduit sleeve being removably connected to one conduit of the pair of conduits, wherein the first and second conduit sleeves each include: a longitudinal extension forming a pathway that extends between a superior opening and an inferior opening, the pathway configured to receive one conduit of the pair of conduits; an inferior extension positioned outside of the pathway and proximate to the inferior opening; and a connection member connected to the inferior extension.

In one form, the inextensible element is a pair of rigidizer arms configured to extend along a contour of the patient's face; and the sleeve is a single sleeve configured to receive both rigidizer arms of the pair of rigidizer arms, the sleeve includes: a superior section, and a pair of inferior sections, wherein each inferior section of the pair of inferior sections is connected to the superior section, wherein each inferior section of the pair of inferior sections includes a longitudinal extension that forms a pathway with an inferior opening, the pathway configured to receive one rigidizer arm of the pair of rigidizer arms, wherein each inferior section of the pair of inferior sections further includes: an inferior extension connected proximate to the inferior opening, the inferior extension being positioned outside of the pathway, and a connection member connected to the inferior extension.

In one form, the inextensible element is a pair of rigidizer arms configured to extend along a contour of the patient's face; and the sleeve is a single sleeve configured to receive both rigidizer arms of the pair of rigidizer arms, the sleeve includes: a superior section that is substantially inextensible; and a pair of inferior sections that are at least partially extensible, wherein each inferior section of the pair of inferior sections is connected to the superior section, wherein each inferior section of the pair of inferior sections includes a longitudinal extension that forms a pathway with an inferior opening, the pathway configured to receive one rigidizer arm of the pair of rigidizer arms, wherein each pathway is isolated from the other pathway.

In some forms: a) the inextensible element is a conduit headgear including a pair of conduits each configured to convey the flow of air at the therapeutic pressure to the plenum chamber; b) each conduit of the pair of conduits includes a tab; c) superior straps of the headgear straps configured to removably connect to the tabs; and/or d) each tab is configured to be positioned superior to the patient's ears, in use.

In some forms: a) the at least one plenum chamber inlet is a pair of plenum chamber inlet ports, the pair of conduits removably connected to the pair of plenum chamber inlet ports; b) each conduit of the pair of conduits includes a clip configured to engage one plenum chamber inlet port of the pair of plenum chamber inlet ports; c) the sleeve includes a longitudinal extension that forms a pathway that extends between a superior opening and an inferior opening, the pathway configured to receive a fluid conduit of the pair of fluid conduits; d) material around the superior opening and/or the inferior opening is elastic and configured to allow the superior opening and/or the inferior opening to stretch and expand a width of the respective opening; e) the material between the superior opening and the inferior opening is substantially inextensible; f) the inferior opening is configured to be positioned proximate to the plenum chamber, in use; and/or g) the superior opening is configured to be positioned at a location inferior to the patient's ear, in use.

In some forms, a) the sleeve includes an inferior extension positioned outside of the pathway and proximate to the inferior opening; b) the inferior extension is more rigid than the pathway; c) the inferior extension is formed with a rigid material; d) the inferior extension is rigidized using a stitching method; e) the inferior extension is substantially inextensible; f) a connection member is connected to the inferior extension; g) the connection member is a magnet; and/or h) the inferior opening and the connection member are oriented in opposite directions.

In some forms, a) the sleeve is a first conduit sleeve, the positioning and stabilising structure further includes a second conduit sleeve having the same structure as the first conduit sleeve, the second conduit sleeve being removably connected to one conduit of the pair of conduits; and/or b) the seal-forming structure is configured to form a seal around the patient's mouth and the patient's nares.

In some forms, a) the inextensible element is a pair of rigidizer arms configured to extend along a contour of the patient's face; b) the plenum chamber inlet port is configured to align with the patient's mouth; c) the plenum chamber further including a pair of arm openings; d) the pair of rigidizer arms being removably connected to the pair of arm openings; e) the pair of rigidizer arms are flexible in one direction and rigid in another direction, the pair of rigidizer arms are configured to bend in order to conform to the shape of the patient's cheeks; f) each rigidizer arm of the pair of rigidizer arms includes a free end and a clip opposite to the free end, the clip configured to engage one arm opening of the pair of arm openings; g) each clip is configured to limit airflow through the respective arm opening; and/or h) the sleeve is a single sleeve configured to receive both rigidizer arms of the pair of rigidizer arms.

In some forms, a) the sleeve includes a superior section; b) the sleeve includes a pair of inferior sections; c) each inferior section of the pair of inferior sections is connected to the superior section; d) each inferior section of the pair of inferior sections includes a longitudinal extension that forms a pathway with an inferior opening; and/or e) the pathway is configured to receive one rigidizer arm of the pair of rigidizer arms.

In some forms, a) each pathway is isolated from the other pathway; b) the superior section is constructed from a different material than the pair of inferior sections; c) the superior section is substantially inextensible and the pair of inferior sections are at least partially extensible; and/or d) material around the inferior opening is elastic and configured to allow the inferior opening to stretch and expand a width of the respective opening.

In some forms, a) the superior section includes a length-adjustable section and is configured to be adjusted based on a size of the patient's head; b) each inferior section of the pair of inferior sections further includes a tab disposed proximate to the superior section and configured to receive a headgear strap; c) each inferior section of the pair of inferior sections includes an inferior extension connected proximate to the inferior opening, the inferior extension being positioned outside of the pathway; d) the inferior extension is more rigid than the pathway; e) the inferior extension is form with a rigid material; f) the inferior extension is rigidized using a stitching method; g) the inferior extension is substantially inextensible; h) a connection member is connected to the inferior extension; i) the connection member is a magnet; and/or j) the connection member and the connection member are oriented in opposite directions.

In some forms, a) the seal-forming structure is configured to form a seal around the patient's mouth and the patient's nares; and/or b) the seal-forming structure is configured to form a seal around the patient's nares and is configured to leave the patient's mouth exposed to ambient.

Another aspect of one form of the present technology comprises a patient interface comprising:
a plenum chamber pressurisable to a therapeutic pressure of at least 4 cmH2O above ambient air pressure, said plenum chamber including a pair of first openings and a second opening, the plenum chamber being structured to receive a flow of air at the therapeutic pressure for breathing by a patient,
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a positioning and stabilising structure to provide a force to hold the seal-forming structure in a therapeutically effective position on the patient's head, the positioning and stabilising structure including:
   at least one inextensible element configured to overlay the patient's cheeks, the at least one inextensible element connected to the pair of first openings,
   a sleeve configured to cover at least a portion of the inextensible element, and
   headgear straps connected to the at least one inextensible element and/or to the sleeve, the headgear straps configured to provide at least a portion of the force;
wherein
the patient interface is configured to allow the patient to breathe from ambient through their mouth in the absence of a flow of pressurised air through the plenum chamber inlet port, or the patient interface is configured to leave the patient's mouth uncovered.

In one form, the inextensible element is a conduit headgear including a pair of conduits each configured to convey the flow of air at the therapeutic pressure to the plenum chamber; and the sleeve includes a longitudinal extension forming a pathway that extends between a superior opening and an inferior opening, the pathway configured to receive a fluid conduit of the pair of fluid conduits, wherein the sleeve further includes: an inferior extension positioned outside of the pathway and proximate to the inferior opening, and a connection member connected to the inferior extension; the headgear straps are directly connected to the connection member and to the conduit headgear.

In one form, the inextensible element is a pair of rigidizer arms configured to extend along a contour of the patient's face, each rigidizer arm of the pair of rigidizer arms is connected to a first opening of the pair of first openings; and the sleeve is a single sleeve configured to receive both rigidizer arms of the pair of rigidizer arms, the sleeve includes: a superior section, and a pair of inferior sections, wherein each inferior section of the pair of inferior sections is connected to the superior section, wherein each inferior section of the pair of inferior sections includes a longitudinal extension that forms a pathway with an inferior opening, the pathway configured to receive one rigidizer arm of the pair of rigidizer arms, wherein each inferior section of the pair of inferior sections includes an inferior extension connected proximate to the inferior opening, the inferior extension being positioned outside of the pathway.

In some forms, the inextensible element is a pair of rigidizer arms configured to extend along a contour of the patient's face, each rigidizer arm of the pair of rigidizer arms is connected to a first opening of the pair of first openings; and the sleeve is a single sleeve configured to receive both rigidizer arms of the pair of rigidizer arms, the sleeve includes, a superior section, and a pair of inferior sections constructed from a different material than the superior section, wherein each inferior section of the pair of inferior sections is connected to the superior section, wherein each inferior section of the pair of inferior sections includes a longitudinal extension that forms a pathway with an inferior opening, the pathway configured to receive one rigidizer arm of the pair of rigidizer arms, each inferior section of the pair of inferior sections further includes a tab disposed proximate to the superior section and configured to receive a headgear strap.

In some forms: a) the inextensible element is a conduit headgear including a pair of conduits each configured to convey the flow of air at the therapeutic pressure to the plenum chamber; b) the headgear straps are directly connected to the conduit headgear; and/or c) the conduit headgear is connected to the pair of first openings with a snap-fit.

In some forms: a) the sleeve includes a longitudinal extension that forms a pathway that extends between a superior opening and an inferior opening; b) the pathway configured to receive a fluid conduit of the pair of fluid conduits; c) material around the superior opening and/or the inferior opening is elastic and configured to allow the superior opening and/or the inferior opening to stretch and expand a width of the respective opening; d) the sleeve includes an inferior extension positioned outside of the pathway and proximate to the inferior opening; e) the inferior extension is more rigid than the pathway; f) the inferior extension is formed with a rigid material; g) the inferior extension is rigidized using a stitching method; and/or h) the inferior extension is substantially inextensible.

In some forms: a) a connection member is connected to the inferior extension; b) the headgear straps are directly connected to the connection member; c) the inferior opening and the connection member are oriented in opposite directions; and/or d) a vent is connected to the second opening and is configured to allow fluid to exit the plenum chamber.

In some forms: a) the inextensible element is a pair of rigidizer arms configured to extend along a contour of the patient's face; b) each rigidizer arm of the pair of rigidizer arms is connected to a first opening of the pair of first openings; c) each rigidizer arm of the pair of rigidizer arms includes a free end and a clip opposite to the free end; d) the clip configured to connect to a first opening of the pair of first openings using a snap-fit; and/or e) the sleeve is a single sleeve configured to receive both rigidizer arms of the pair of rigidizer arms.

In some forms: a) the sleeve includes a superior section; b) the sleeve includes a pair of inferior sections; c) each inferior section of the pair of inferior sections is connected to the superior section; d) each inferior section of the pair of inferior sections includes a longitudinal extension that forms a pathway with an inferior opening; and/or f) the pathway configured to receive one rigidizer arm of the pair of rigidizer arms.

In some forms: a) each pathway is isolated from the other pathway; b) the superior section is constructed from a different material than the pair of inferior sections; c) each inferior section of the pair of inferior sections further includes a tab disposed proximate to the superior section and configured to receive a headgear strap; d) each inferior section of the pair of inferior sections includes an inferior extension connected proximate to the inferior opening, the inferior extension being positioned outside of the pathway; e) the inferior extension is more rigid than the pathway; f) a connection member connected to the inferior extension; and/or g) the headgear straps are directly connected to the connection member.

Another aspect of one form of the present technology comprises a method comprising:
providing a plenum chamber pressurisable to a therapeutic pressure of at least 6 cmH2O above ambient air pressure, said plenum chamber including a pair of first openings and a second opening;
providing a first inextensible member and a second inextensible member, the first inextensible member being a conduit headgear, and the second inextensible member being a pair of rigidizer arms;
providing a first sleeve useable with the first inextensible member and a second sleeve usable with the second inextensible member;
selecting one of the first inextensible member and the second inextensible member;
selecting a corresponding one of the first sleeve and the second sleeve;
connecting the selecting one of the first inextensible member and the second inextensible member to the corresponding one of the first sleeve and the second sleeve; and
connecting the selected one of the first inextensible member and the second inextensible member to the pair of first openings of the plenum chamber.

In some forms the selected one of the first inextensible member and the second inextensible member to the pair of first openings of the plenum chamber using a snap-fit.

Additional steps may include: a) providing first headgear straps useable with the first inextensible member and second headgear straps usable with the second inextensible member; and/or b) connecting the selected one of the first headgear straps and the second headgear straps directly to the selected one of the selecting one of the first inextensible member and the second inextensible member and/or to the selected one of the first inextensible member and the second inextensible member.

Additional steps may include: a) providing a vent usable with the first inextensible member and a conduit tube usable with the second inextensible member, and connecting one of the conduit tube and the vent to the second opening; and/or b) the plenum chamber is a first plenum chamber, the method further comprising providing a second plenum chamber, and selecting one of the first plenum chamber and the second plenum chamber.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

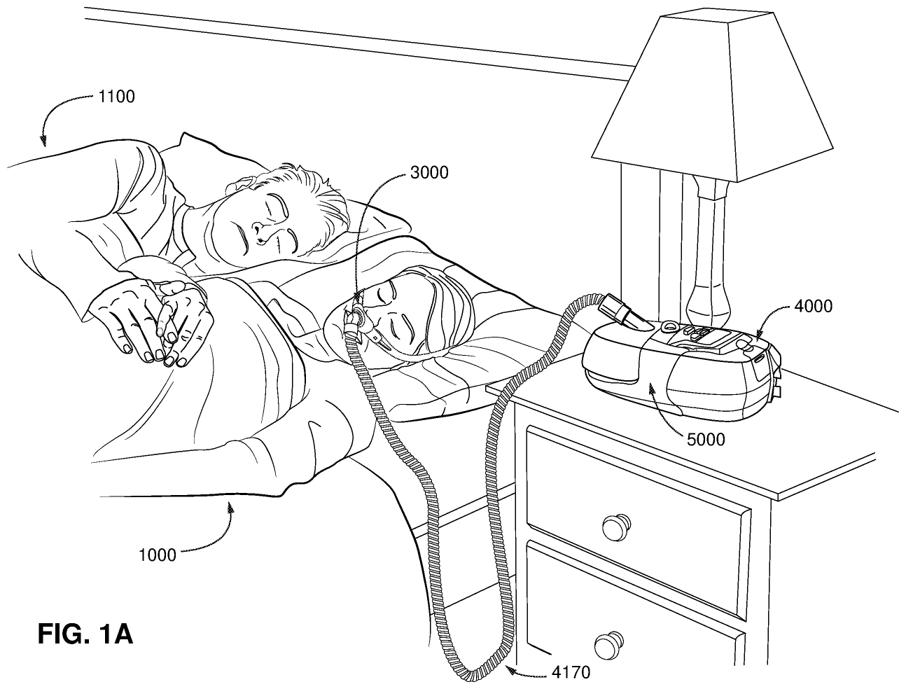

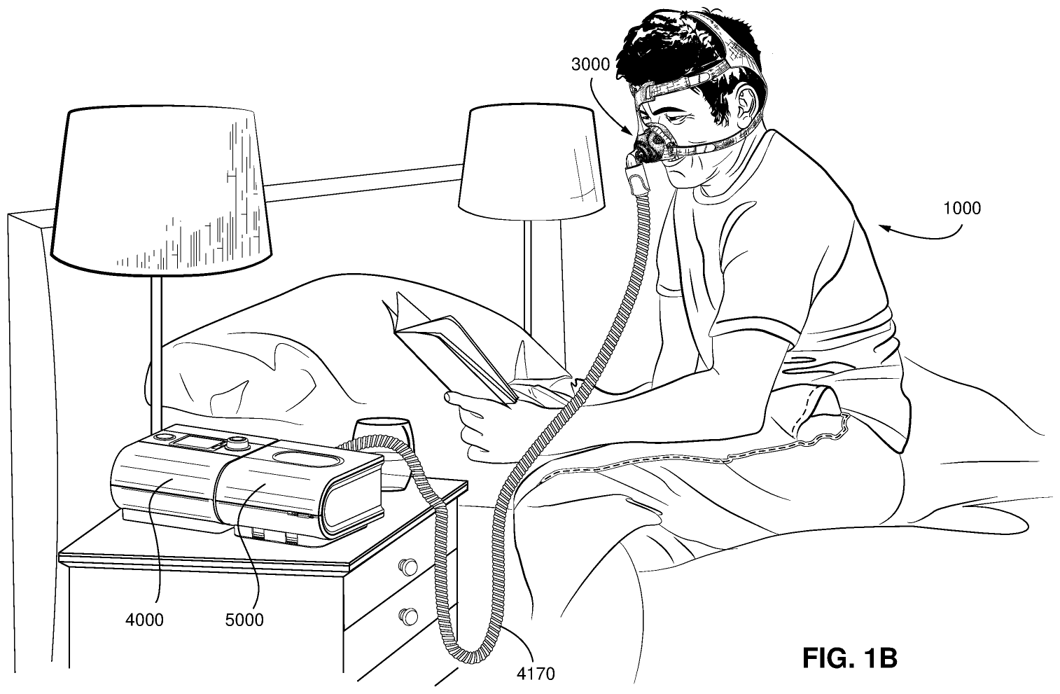

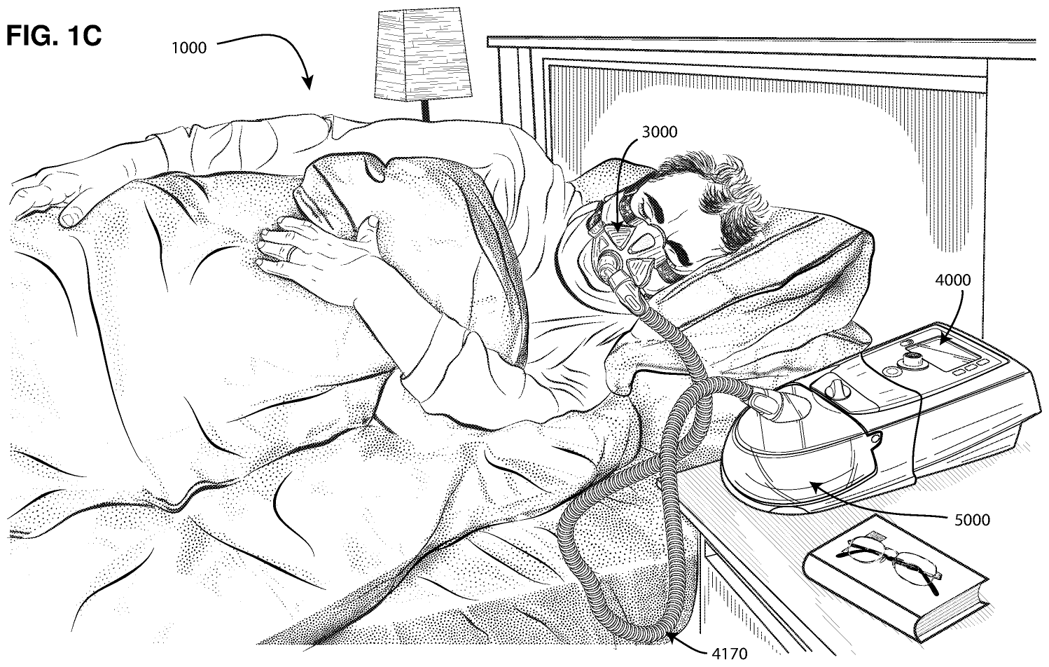

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

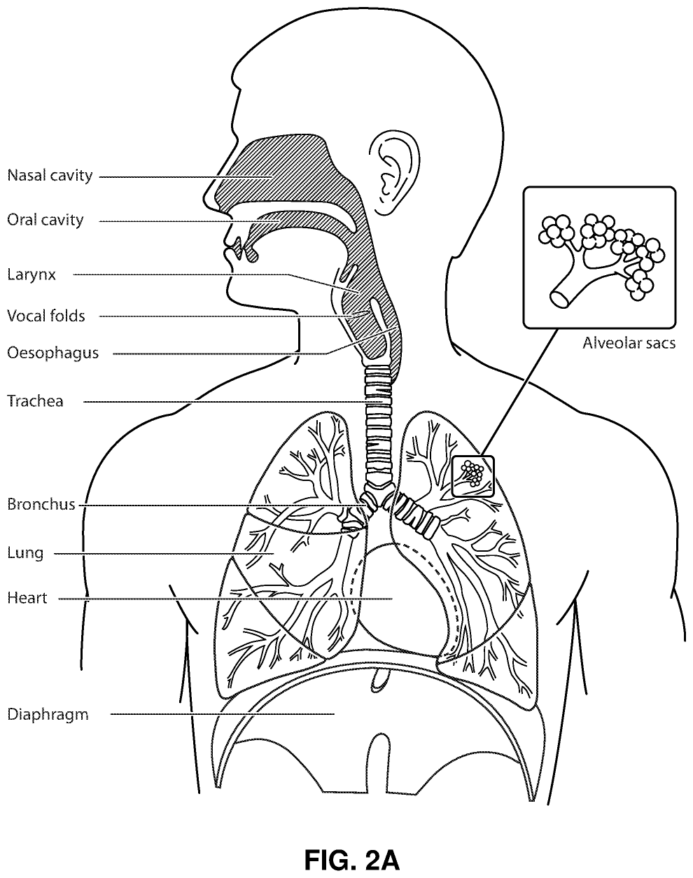

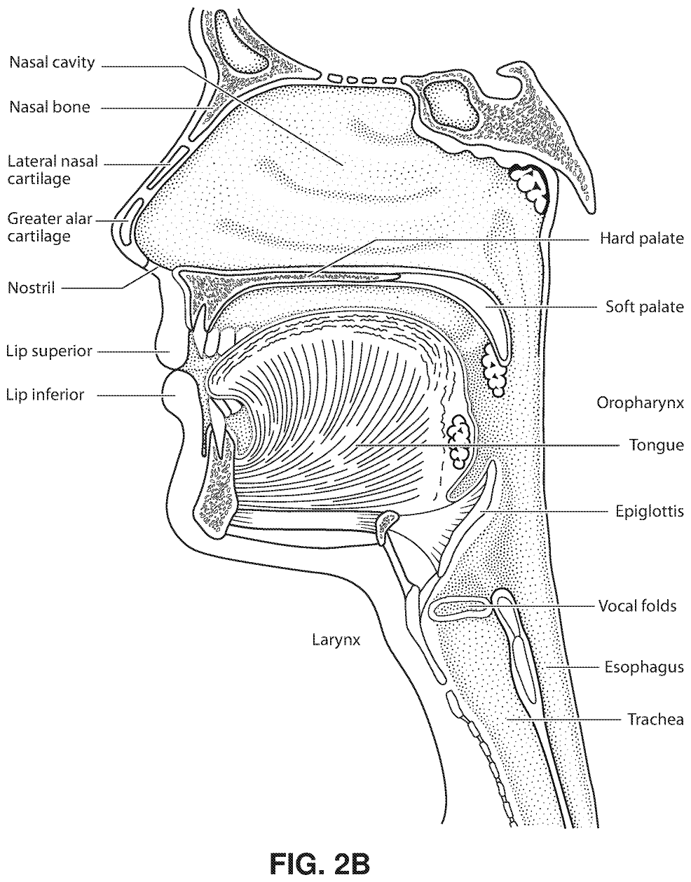

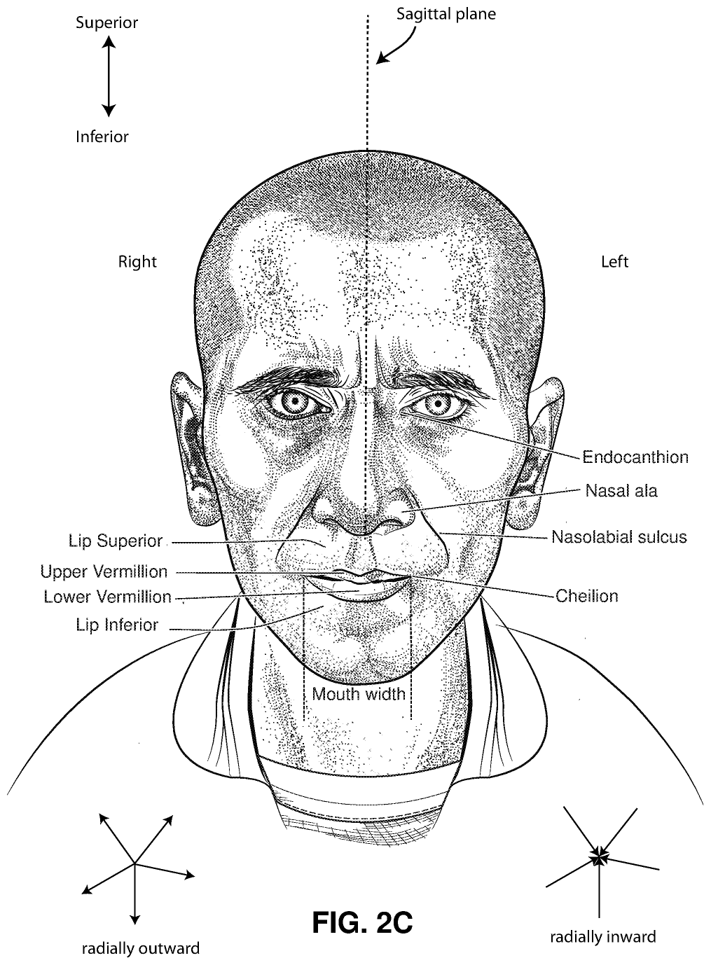

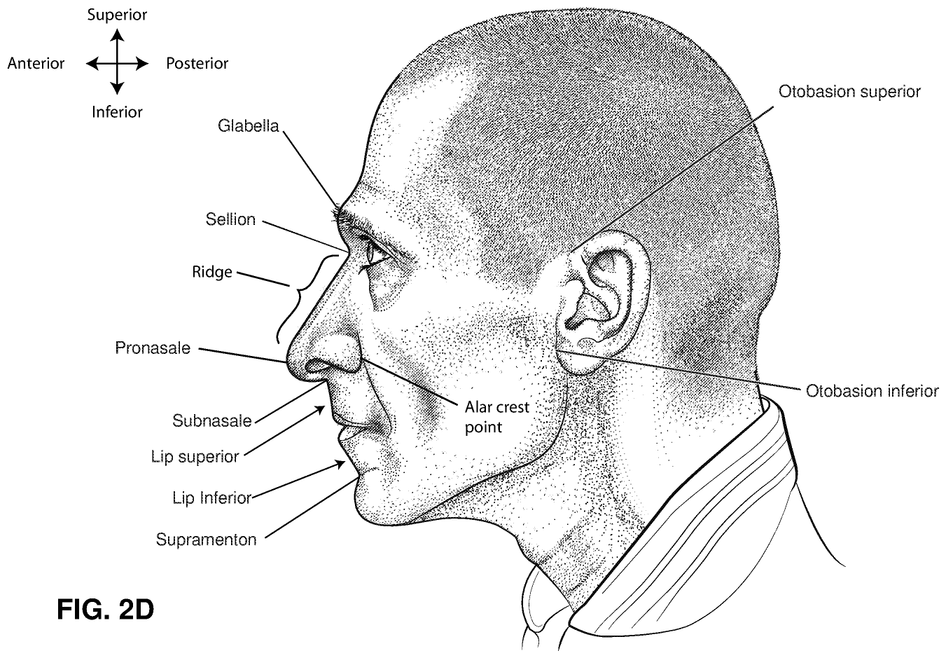

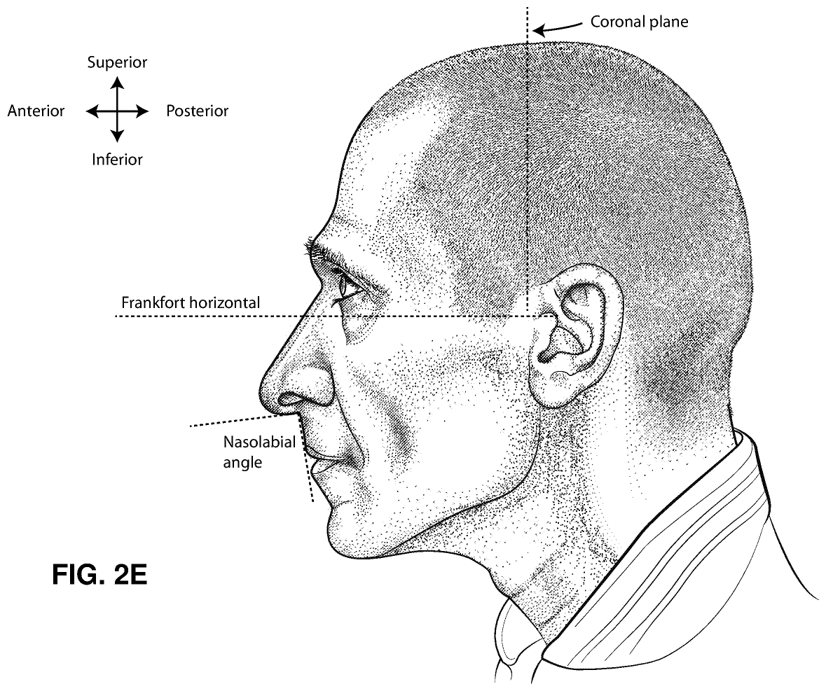

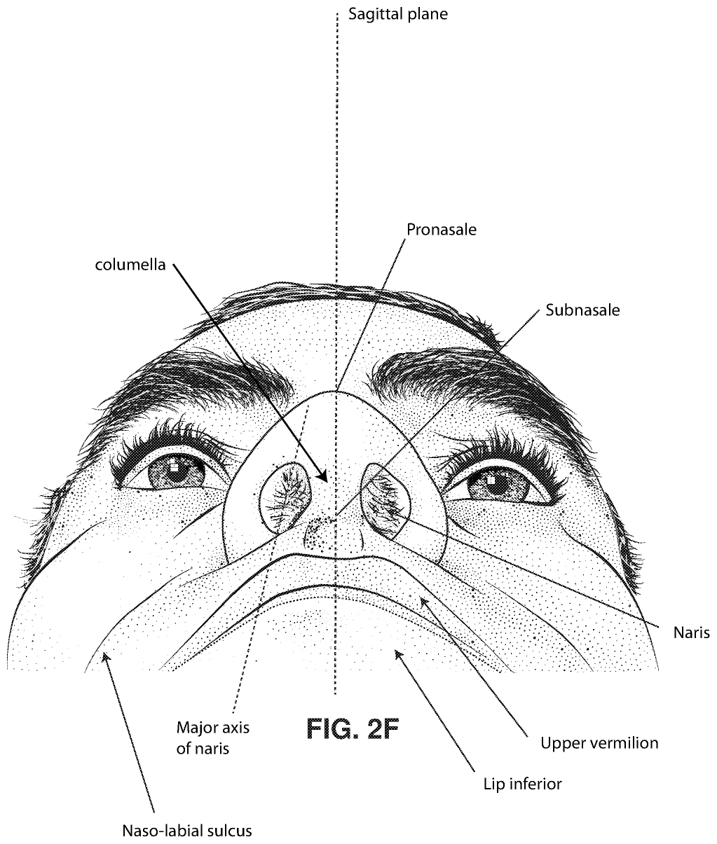

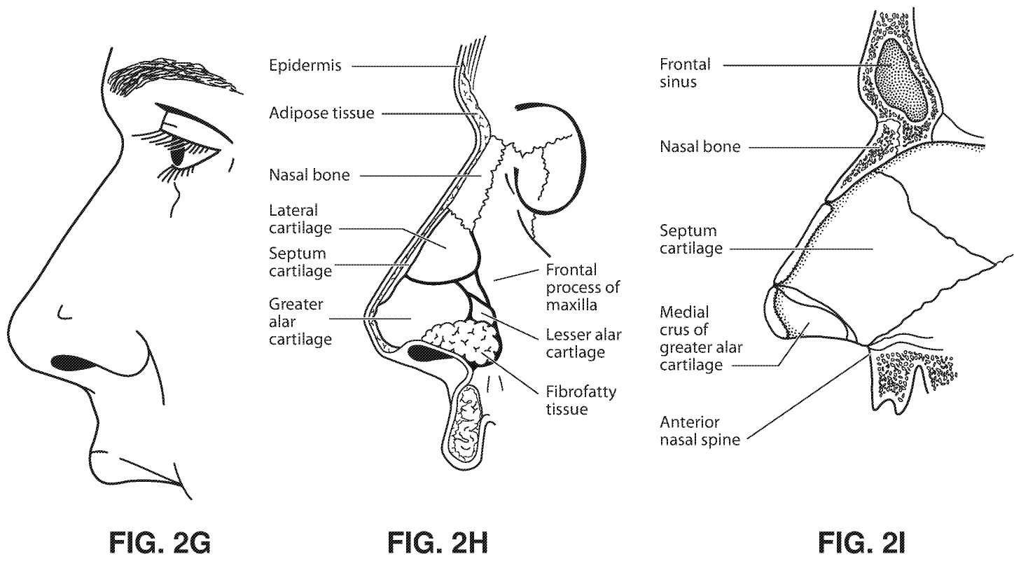

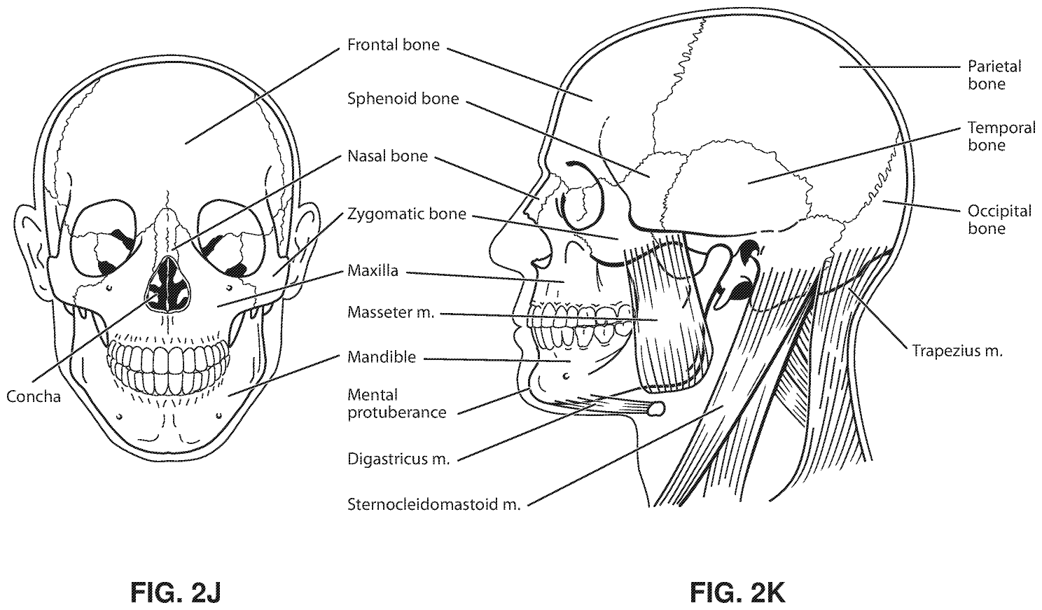

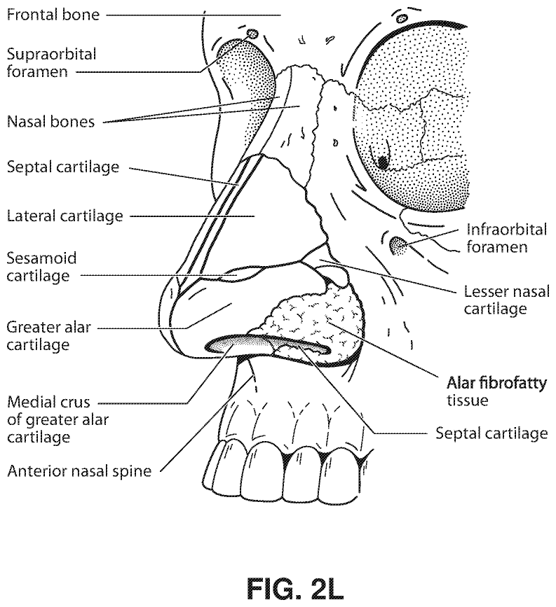

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

### 4.3 Patient Interface

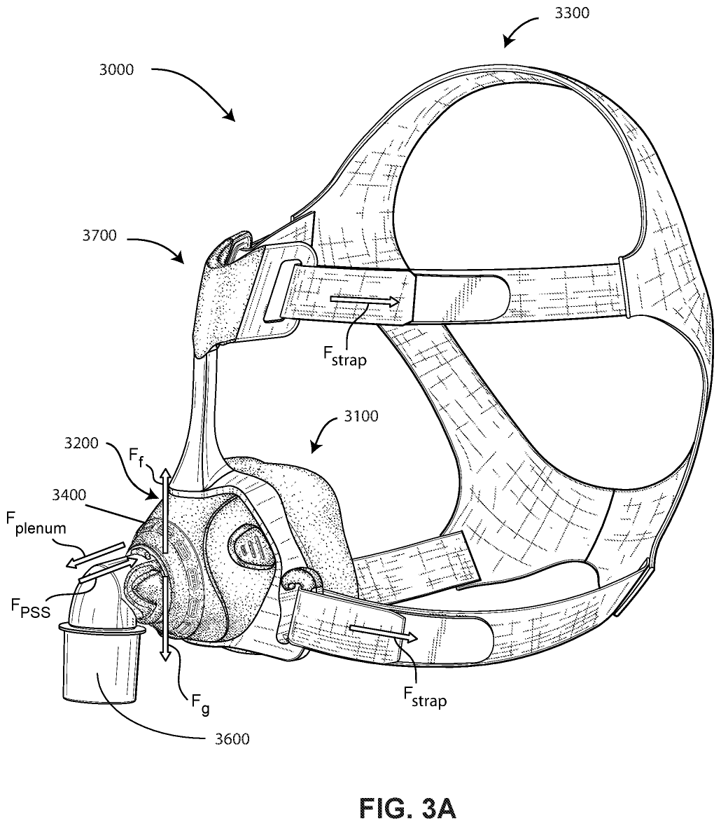

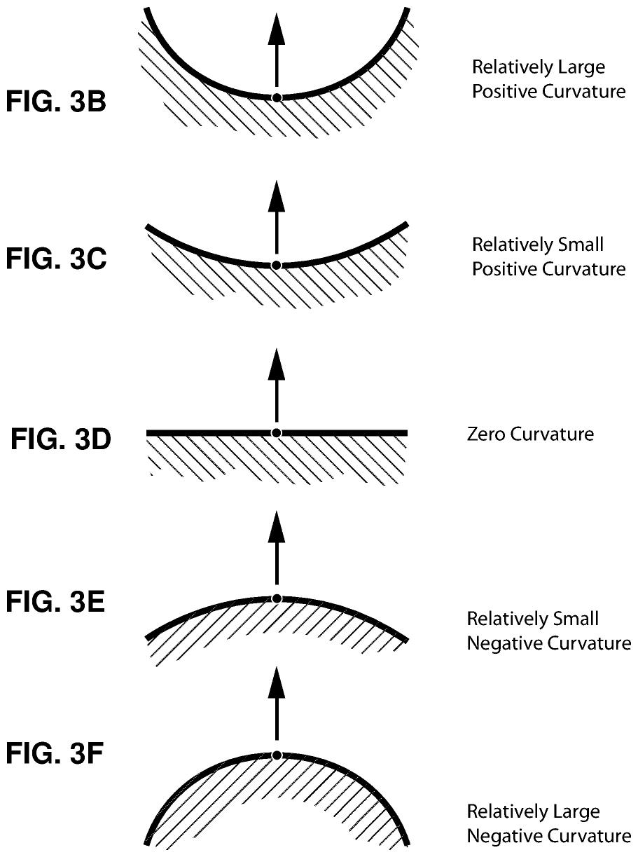

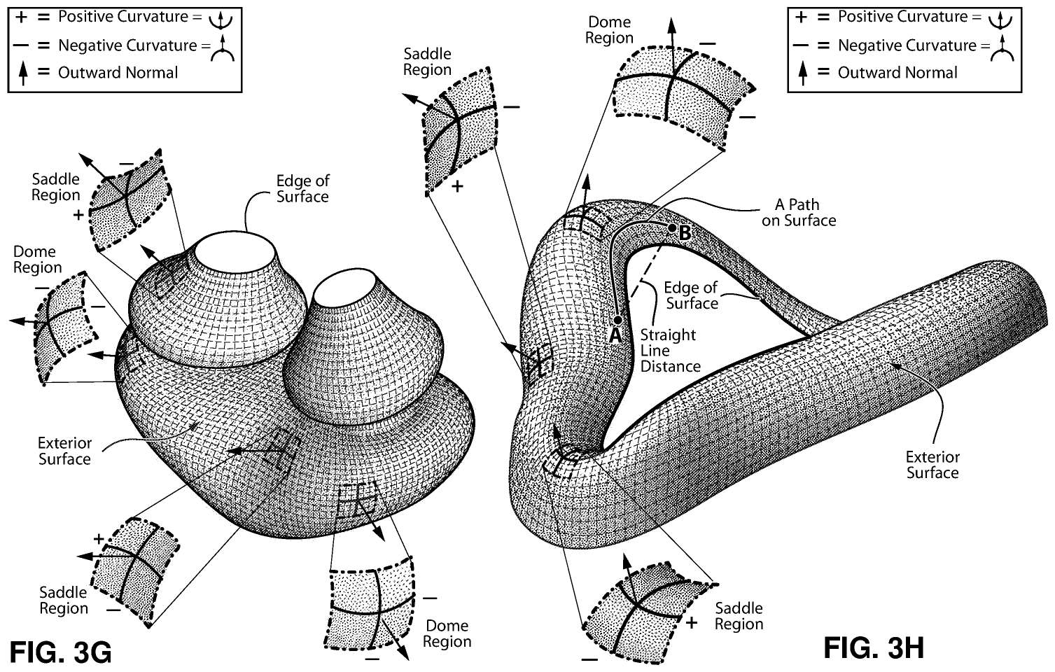

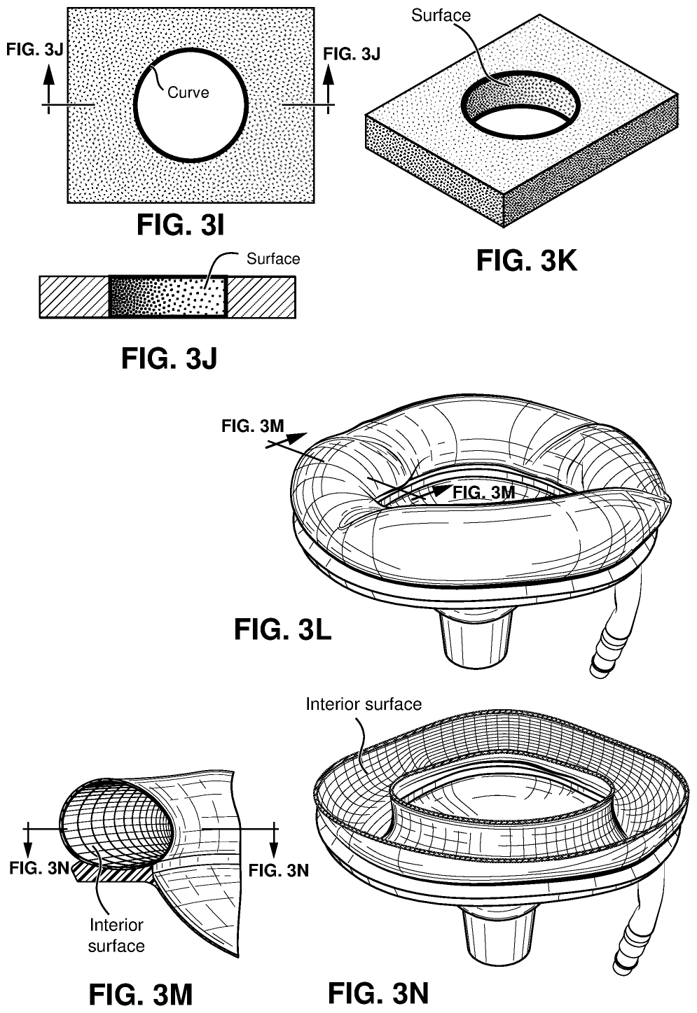

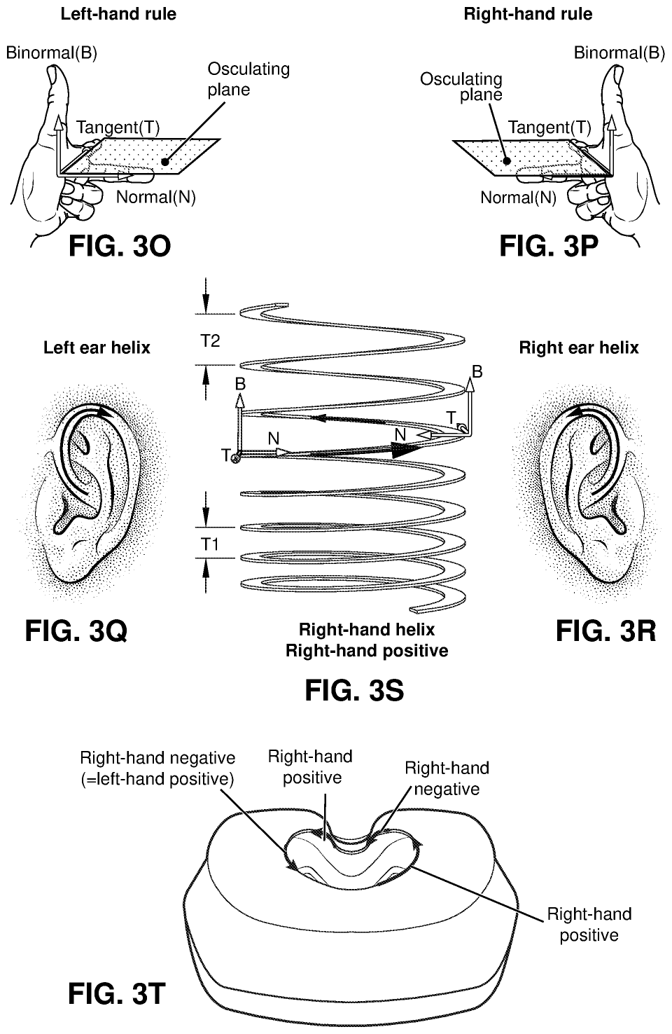

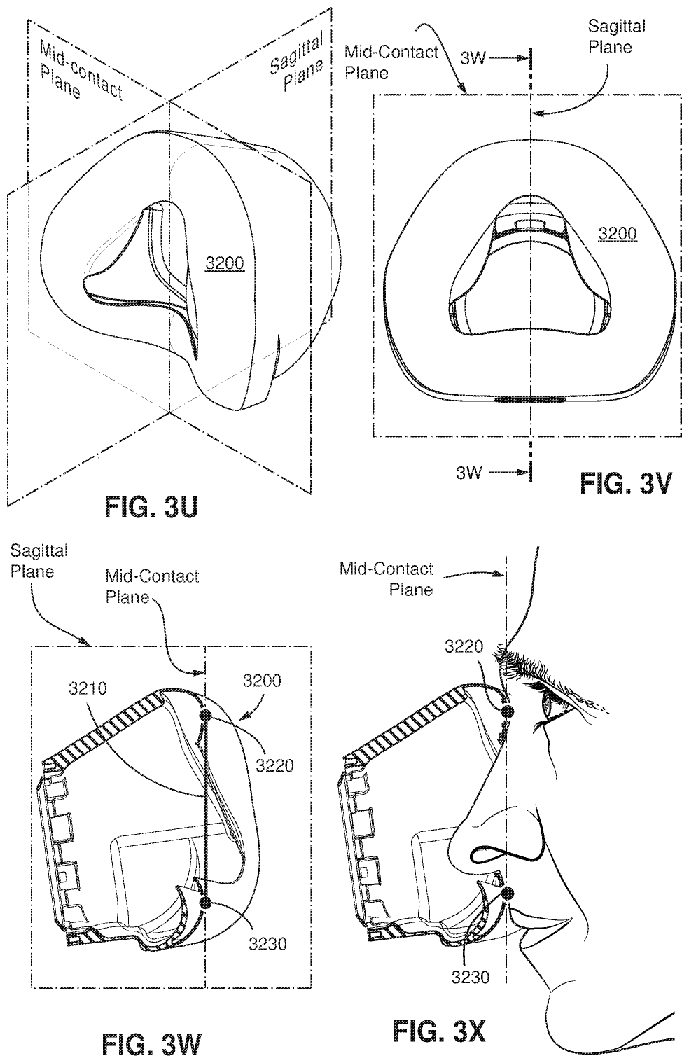

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

### 4.4 RPT Device

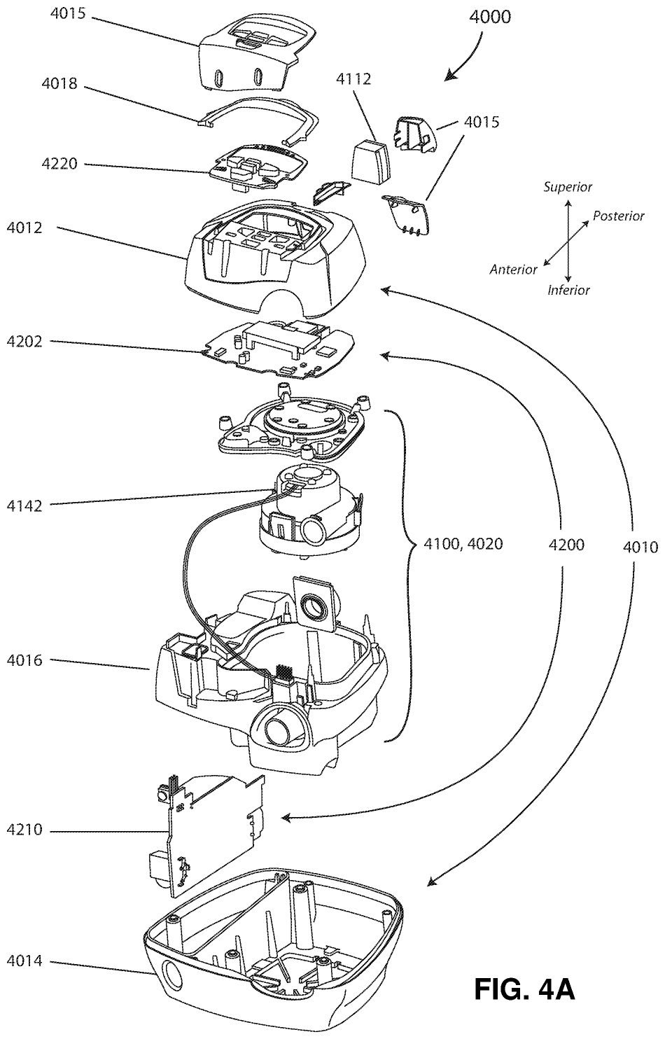

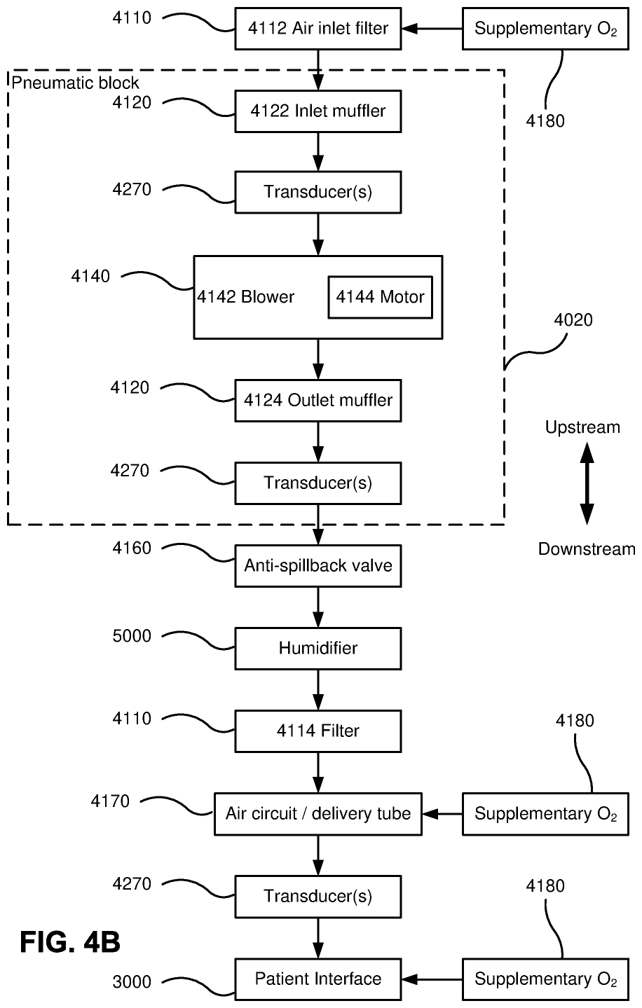

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 4.5 Humidifier

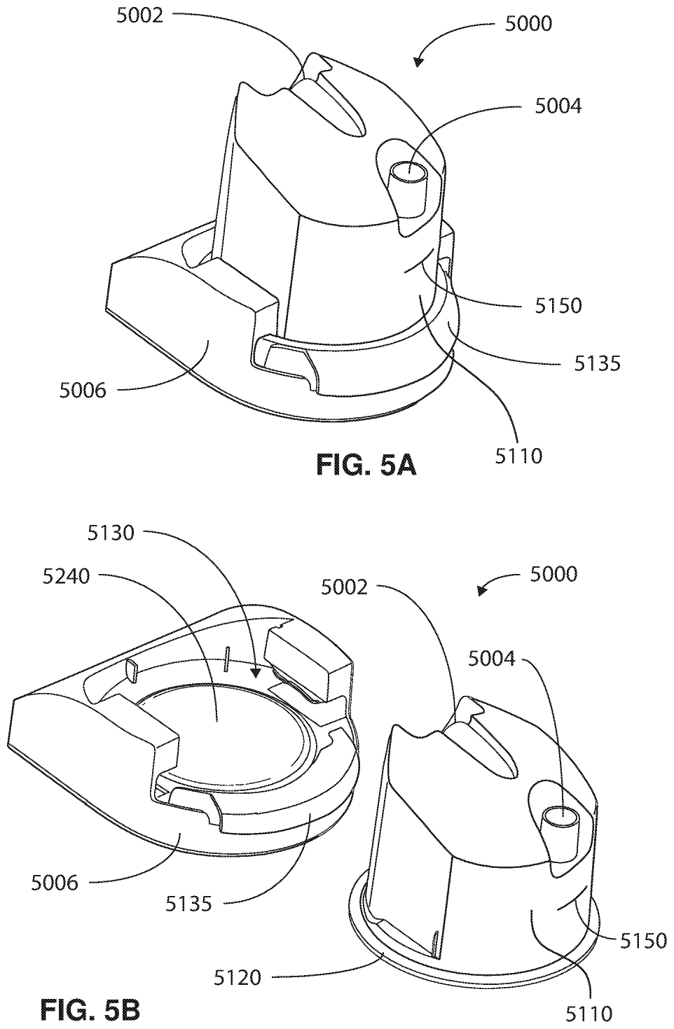

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Breathing Waveforms

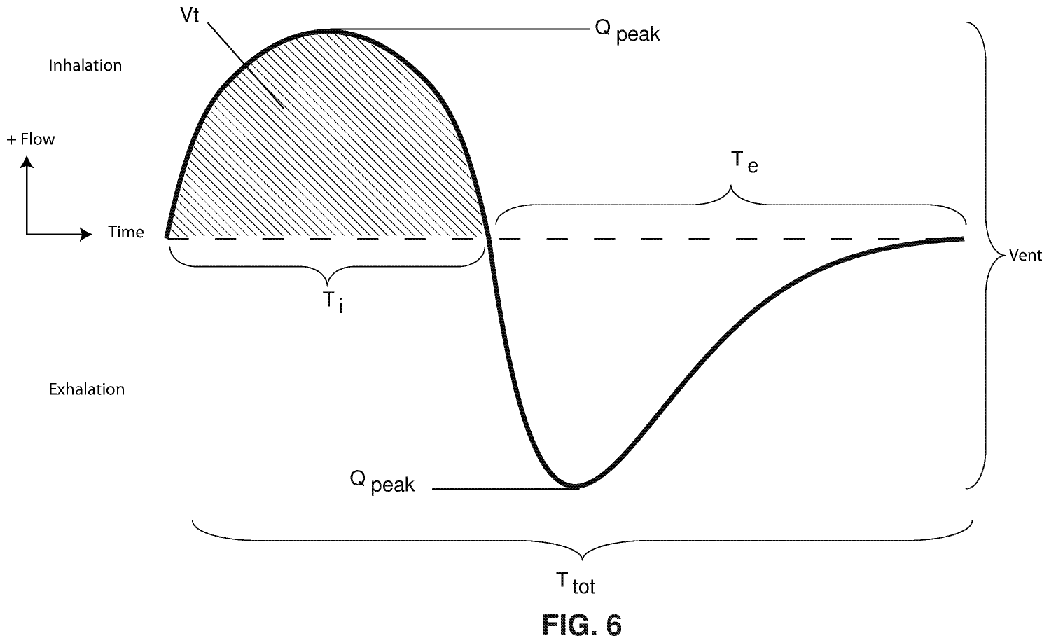

FIG. 6 shows a model typical breath waveform of a person while sleeping.

### 4.7 Modular Aspects

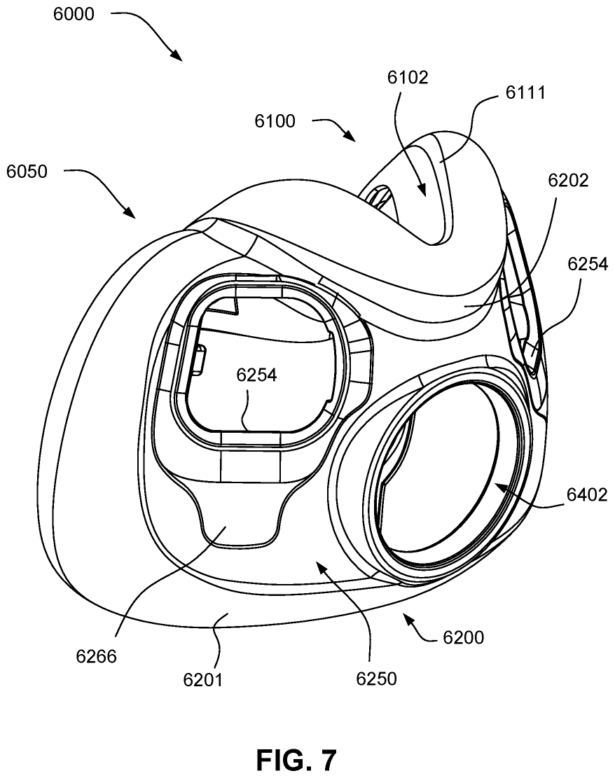

FIG. 7 shows a perspective view of a cushion of a patient interface configured to be worn by a patient and convey pressurized air to the patient's nose and the patient's mouth.

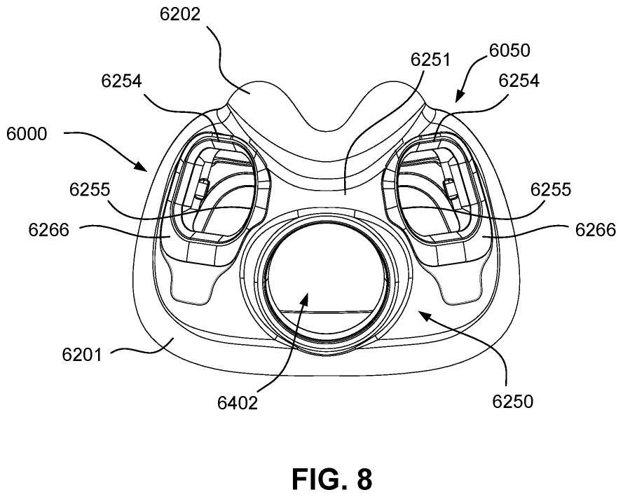

FIG. 8 shows a front view of the cushion of FIG. 7.

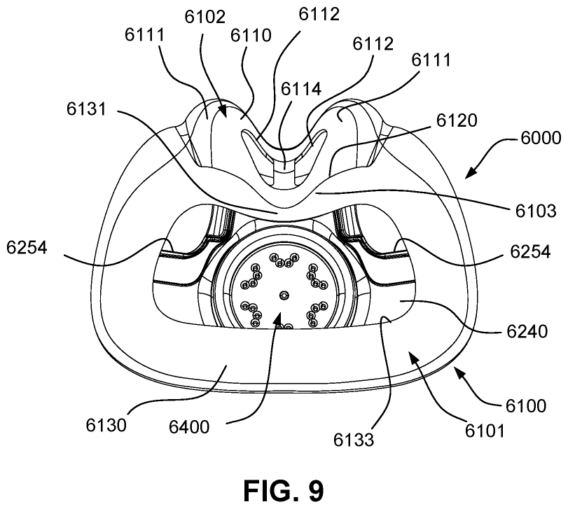

FIG. 9 shows a rear view of the cushion of FIG. 7.

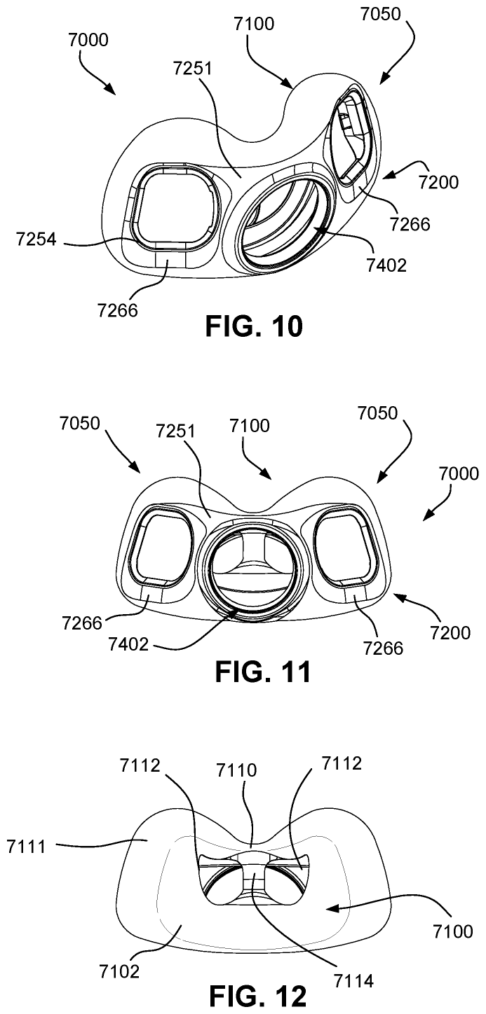

FIG. 10 shows a perspective view of a cushion of a patient interface configured to be worn by a patient and convey pressurized air to the patient's nose.

FIG. 11 shows a front view of the cushion of FIG. 10.

FIG. 12 shows a rear view of the cushion of FIG. 10.

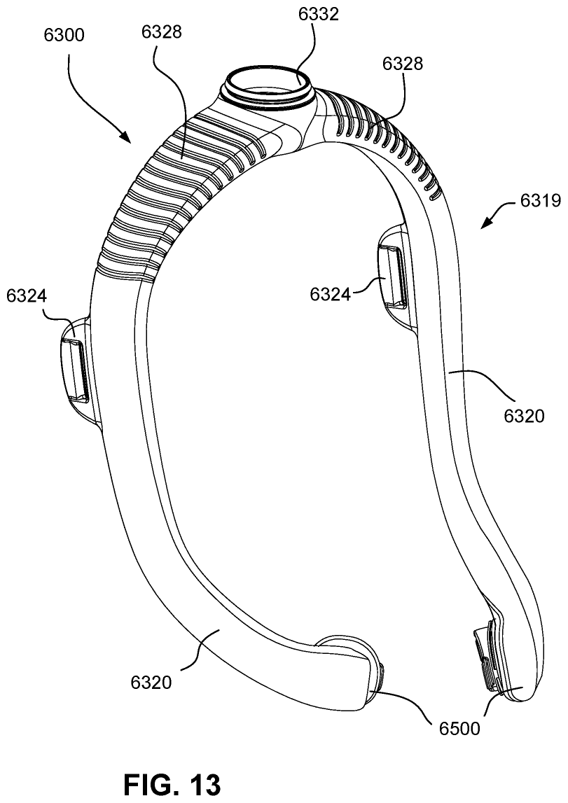

FIG. 13 shows a perspective view of conduit headgear usable with either the cushion of FIG. 7 or the cushion of FIG. 10.

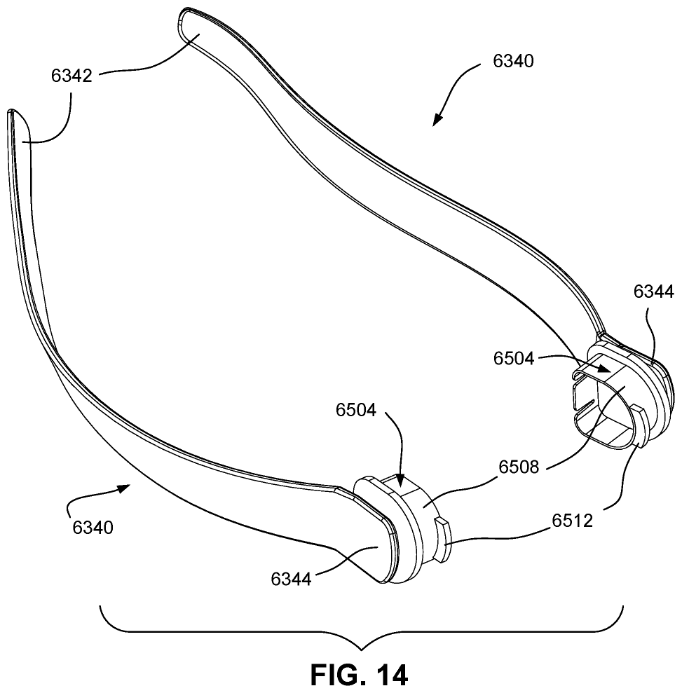

FIG. 14 shows a perspective view of rigidizer arms usable with either the cushion of FIG. 7 or the cushion of FIG. 10.

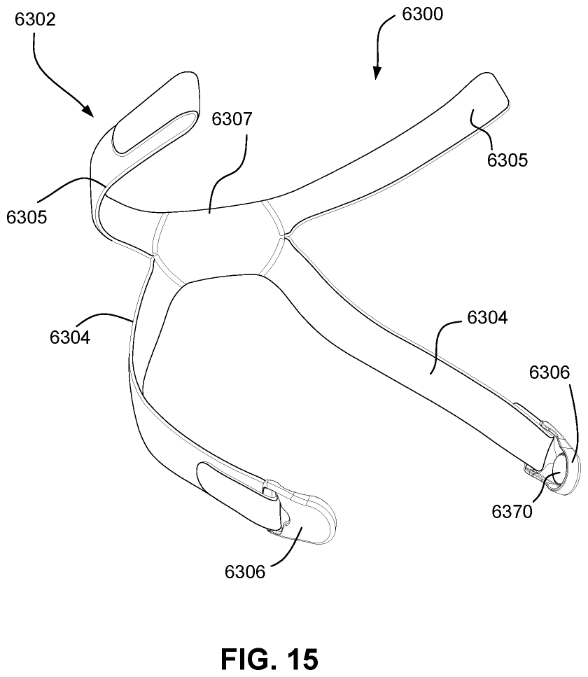

FIG. 15 shows a perspective view of headgear straps usable with the cushion of FIG. 7.

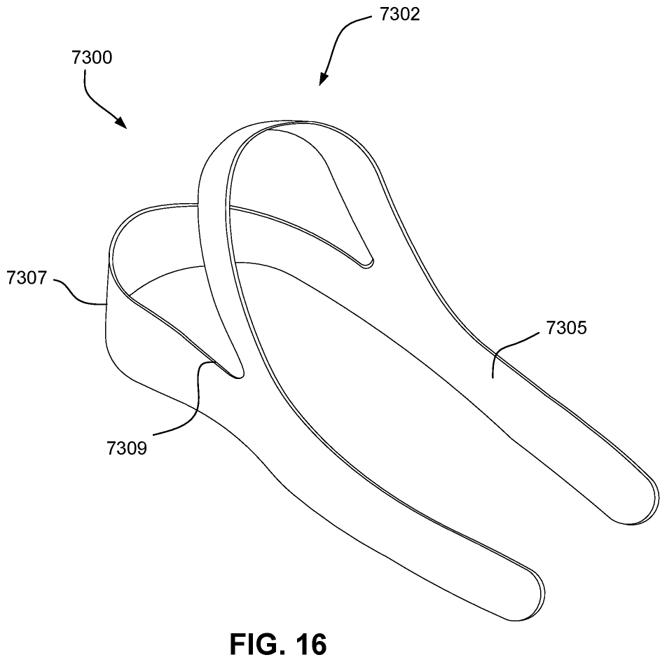

FIG. 16 shows a perspective view of headgear straps usable with the cushion of FIG. 10.

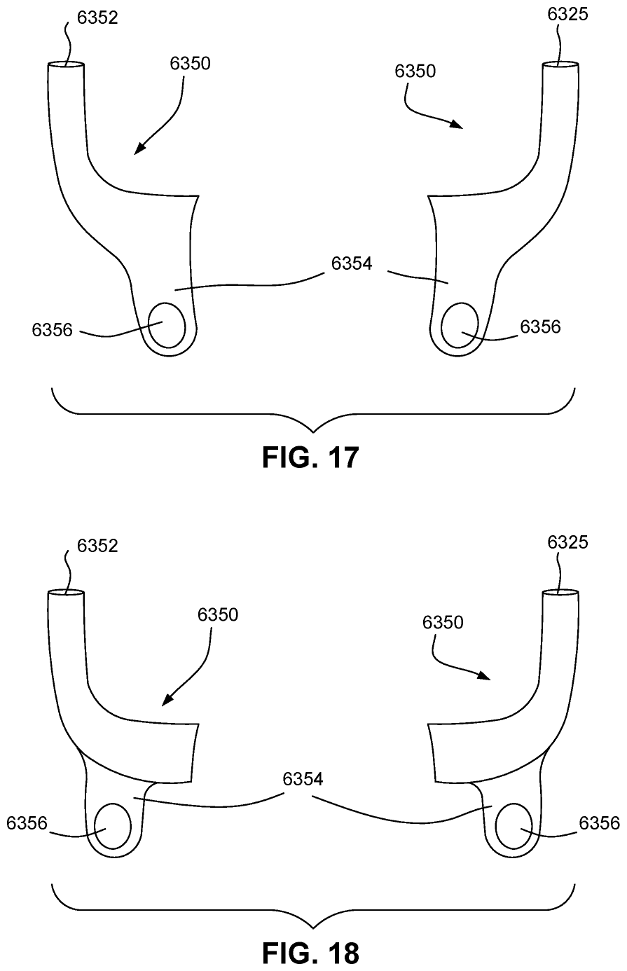

FIG. 17 shows a front view of a pair of sleeves that is removably fitted to either the conduit headgear of FIG. 13 or the rigidizer arms of FIG. 14.

FIG. 18 shows a front view of an alternate pair of sleeves that is removably fitted to either the conduit headgear of FIG. 13 or the rigidizer arms of FIG. 14.

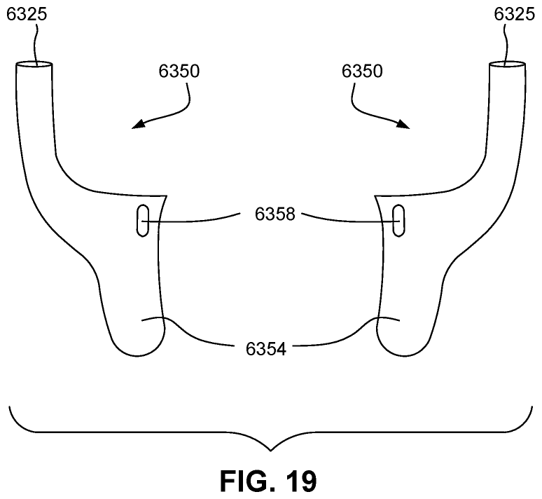

FIG. 19 shows a rear view of the pair of sleeves of FIG. 17.

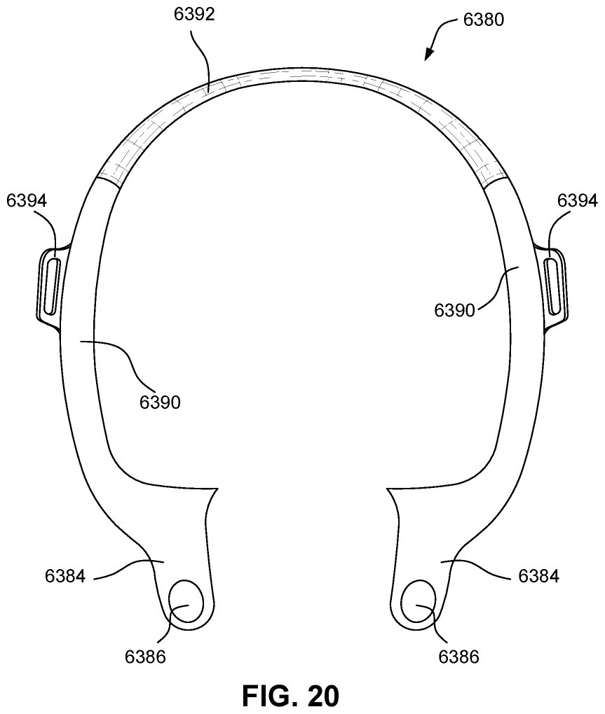

FIG. 20 shows a front view of a full sleeve that is removably fitted to the rigidizer arms of FIG. 14.

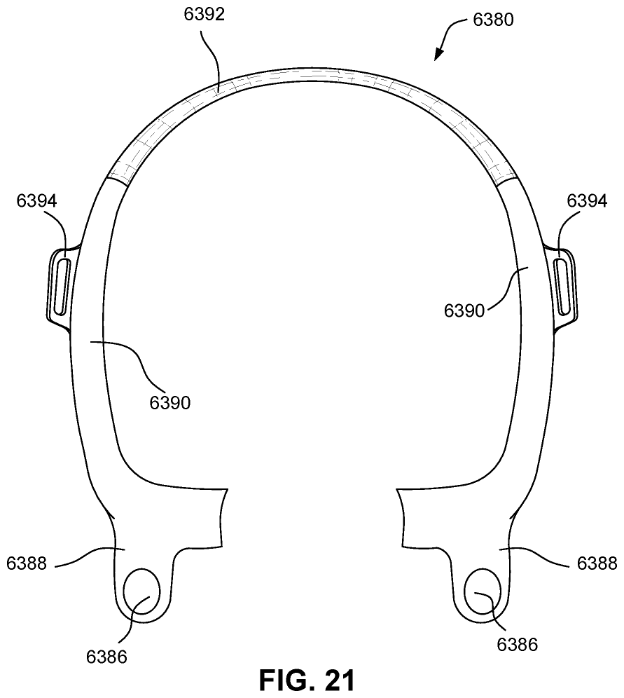

FIG. 21 shows a front view of an alternate full sleeve that is removably fitted to the rigidizer arms of FIG. 14.

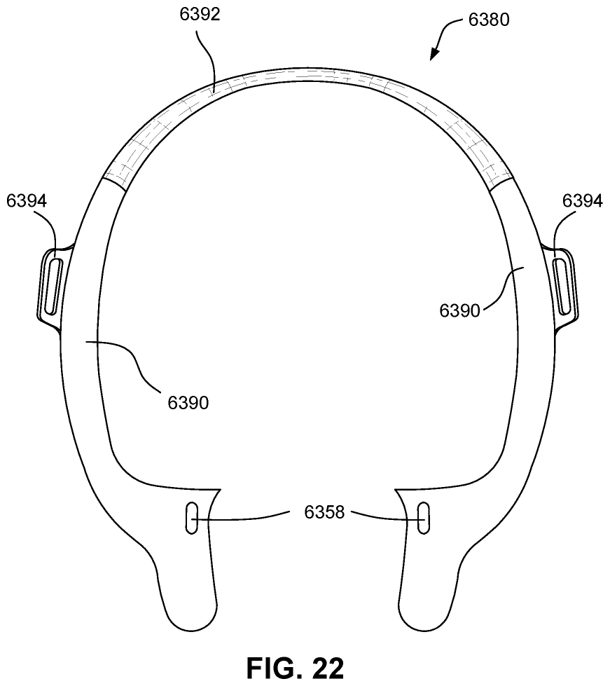

FIG. 22 shows a rear view of the full sleeve of FIG. 20.

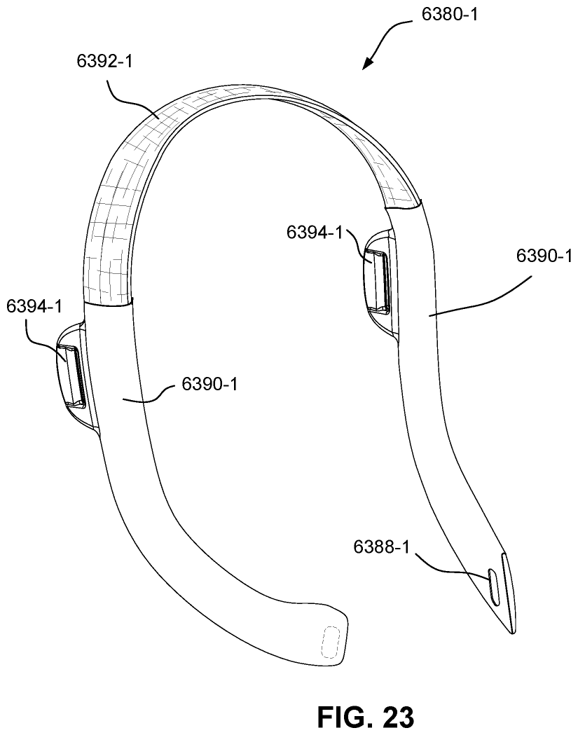

FIG. 23 shows a front perspective view of yet another alternate form of a full sleeve that is removably fitted to the rigidizer arms of FIG. 14.

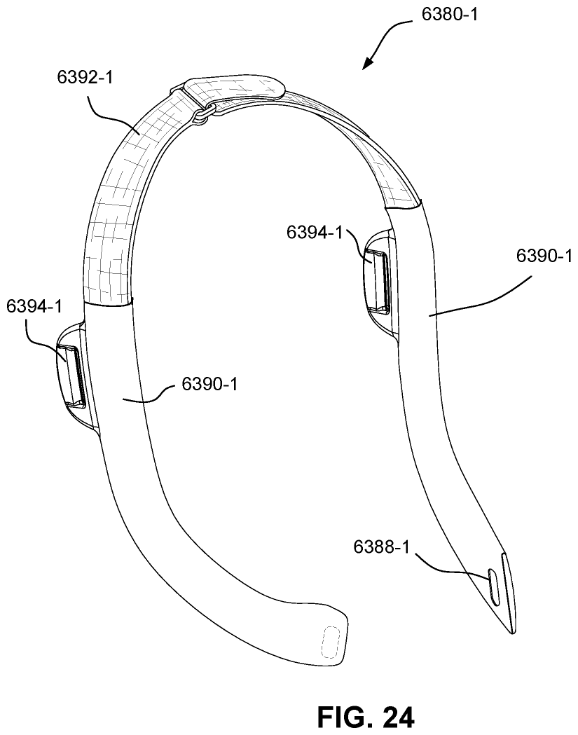

FIG. 24 shows a front perspective view of an alternate version of the full sleeve of FIG. 23.

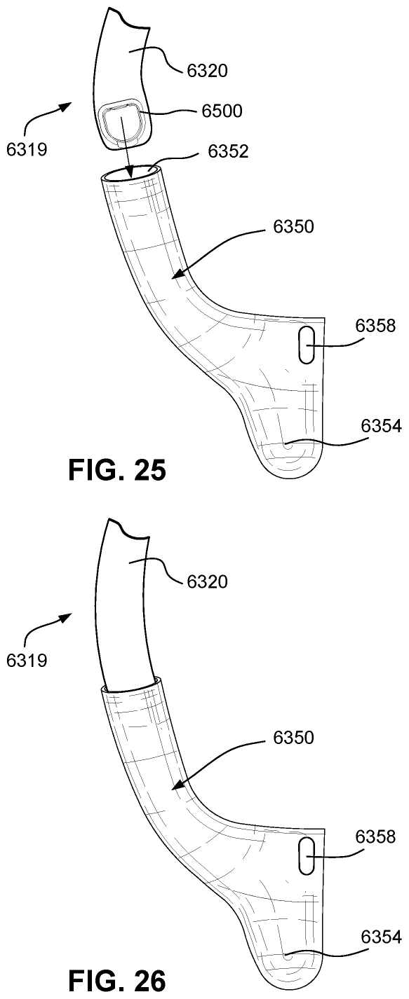

FIG. 25 shows a first step in connecting the sleeve of FIG. 17 to the conduit headgear of FIG. 13 where the sleeve and the conduit headgear are not in contact.

FIG. 26 shows a second step in connecting the sleeve of FIG. 17 to the conduit headgear of FIG. 13 where the conduit headgear is initially slid into the sleeve.

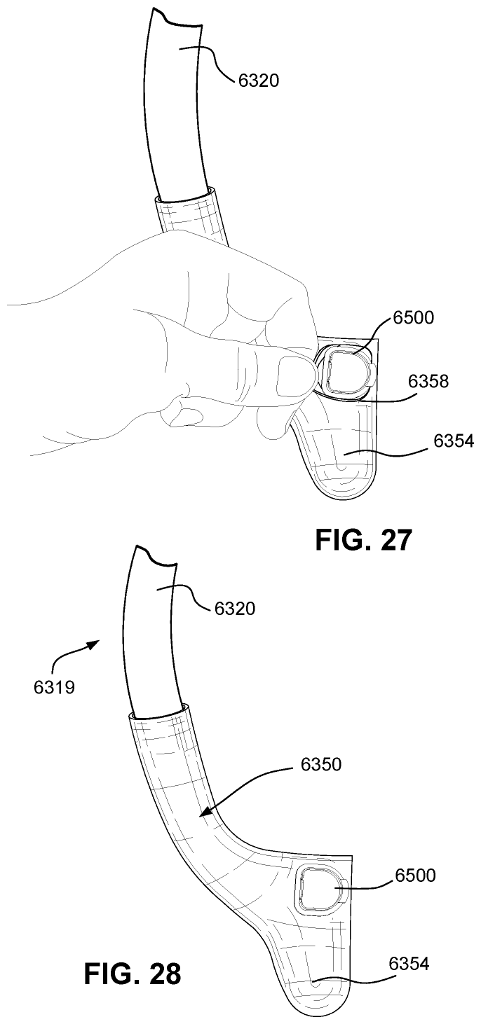

FIG. 27 shows a third step in connecting the sleeve of FIG. 17 to the conduit headgear of FIG. 13 where an end of the conduit headgear is positioned through an opening in the sleeve.

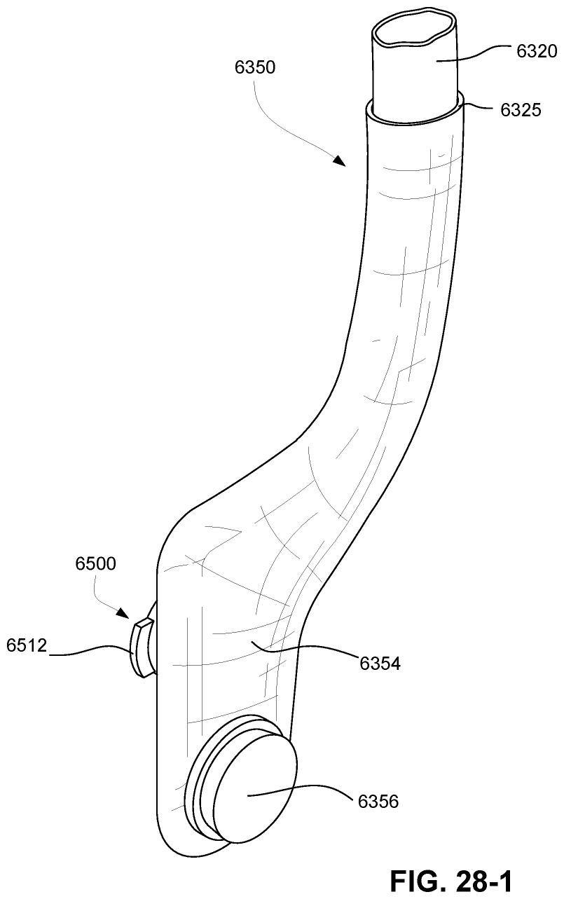

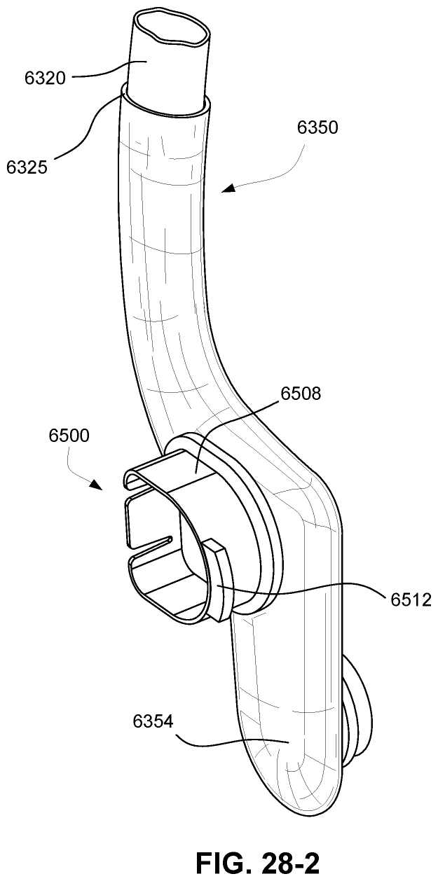

FIG. 28 shows a fourth step in connecting the sleeve of FIG. 17 to the conduit headgear of FIG. 13 where the sleeve is fully connected to the conduit headgear.

FIG. 28-1 shows a front perspective view of the sleeve of FIG. 17 connected to the conduit headgear of FIG. 13.

FIG. 28-2 shows a rear perspective view of the sleeve of FIG. 17 connected to the conduit headgear of FIG. 13.

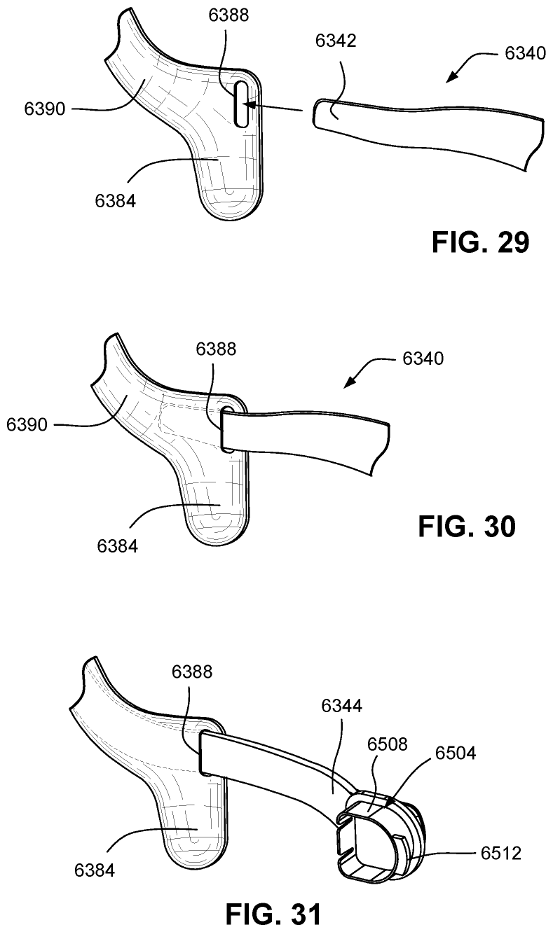

FIG. 29 shows a first step in connecting the sleeve of FIG. 20 to the rigidizer arms of FIG. 14 where the sleeve and the arm are not in contact.

FIG. 30 shows a second step in connecting the sleeve of FIG. 20 to the rigidizer arm of FIG. 14 where the arm is initially slid into the sleeve.

FIG. 31 shows a third step in connecting the sleeve of FIG. 20 to the rigidizer arm of FIG. 14 where the arm is further slid into the sleeve.

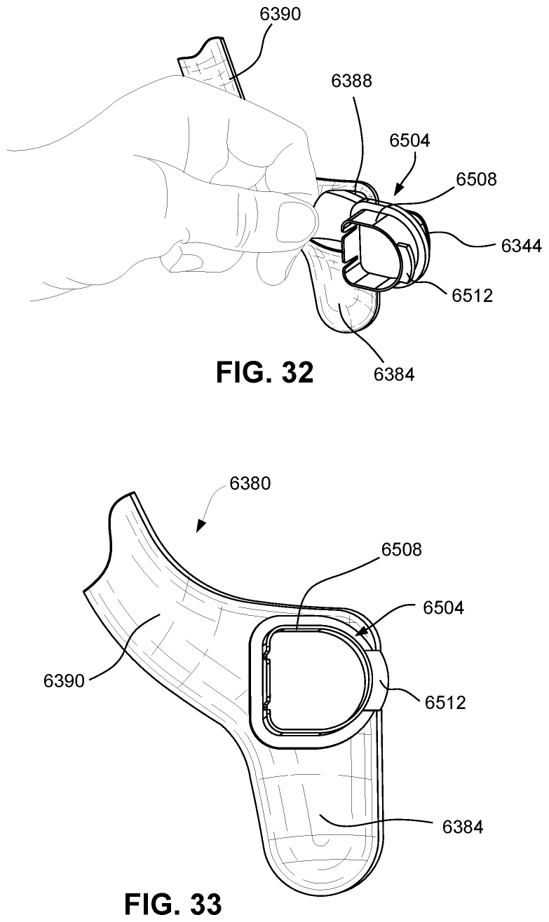

FIG. 32 shows a fourth step in connecting the sleeve of FIG. 20 to the rigidizer arm of FIG. 14 where an end of the rigidizer arm is positioned through an opening in the sleeve.

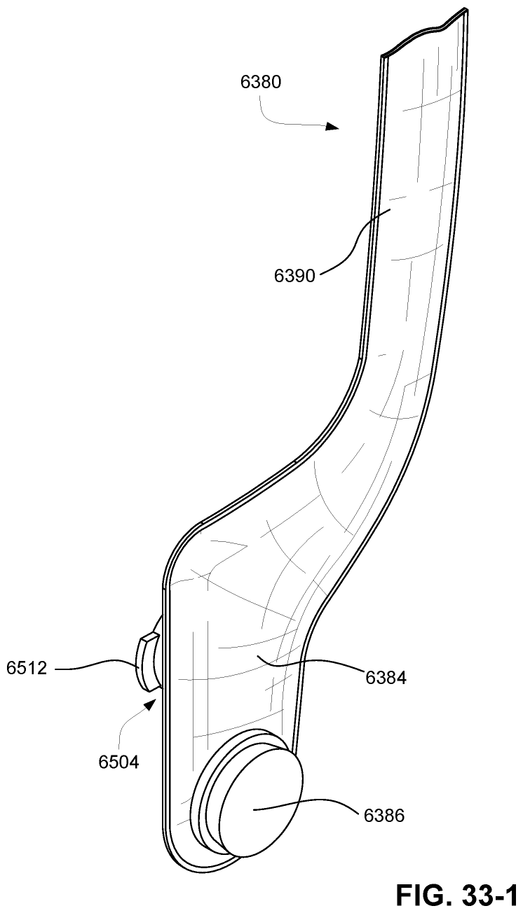

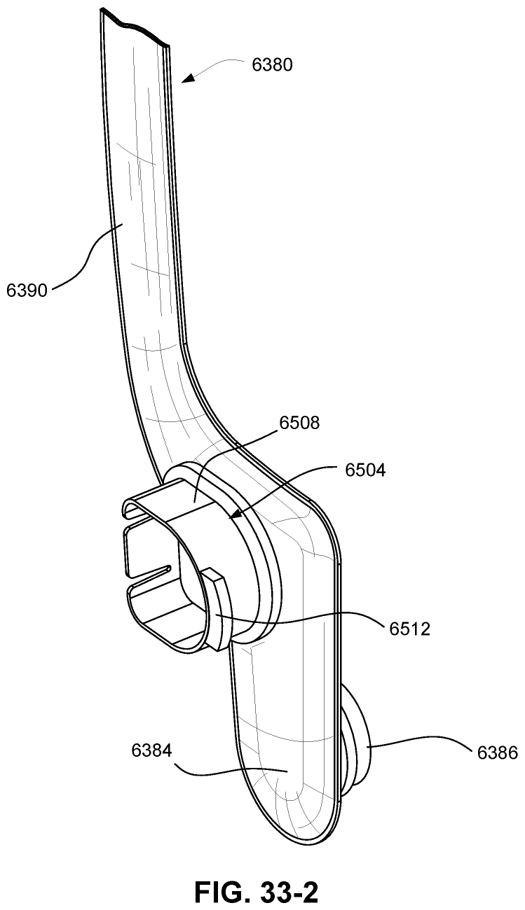

FIG. 33 shows a fifth step in connecting the sleeve of FIG. 20 to the rigidizer arm of FIG. 14 where the sleeve is fully connected to the conduit headgear.

FIG. 33-1 shows a front perspective view of the sleeve of FIG. 20 to the rigidizer arm of FIG. 14.

FIG. 33-2 shows a rear perspective view of the sleeve of FIG. 20 to the rigidizer arm of FIG. 14.

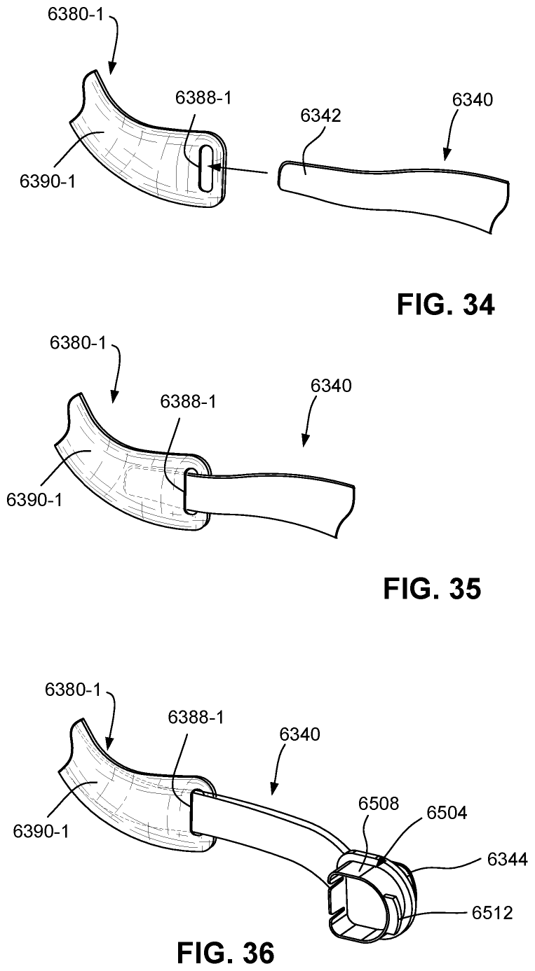

FIG. 34 shows a first step in connecting the sleeve of FIG. 23 to the rigidizer arms of FIG. 14 where the sleeve and the arm are not in contact.

FIG. 35 shows a second step in connecting the sleeve of FIG. 23 to the rigidizer arm of FIG. 14 where the arm is initially slid into the sleeve.

FIG. 36 shows a third step in connecting the sleeve of FIG. 23 to the rigidizer arm of FIG. 14 where the arm is further slid into the sleeve.

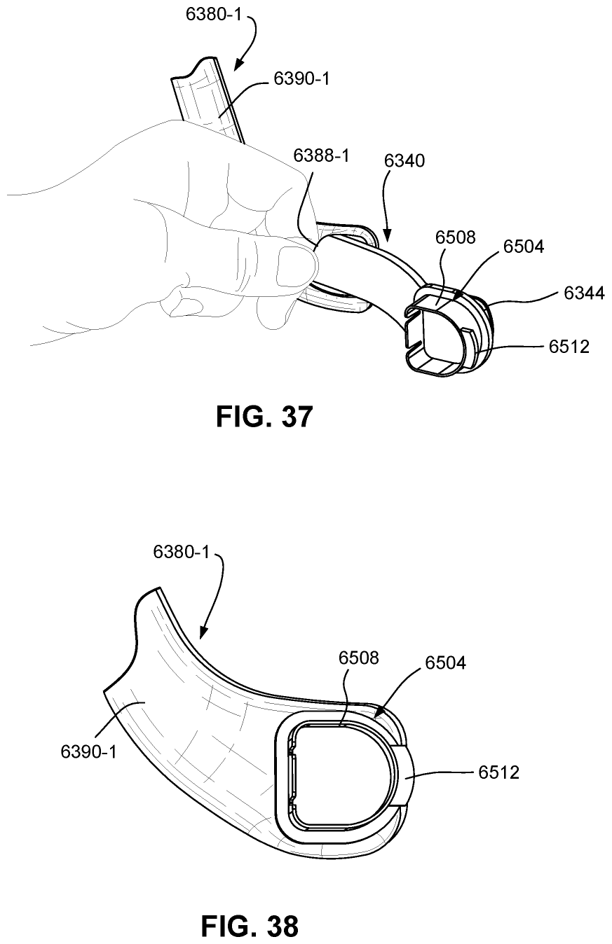

FIG. 37 shows a fourth step in connecting the sleeve of FIG. 23 to the rigidizer arm of FIG. 14 where an end of the rigidizer arm is positioned through an opening in the sleeve.

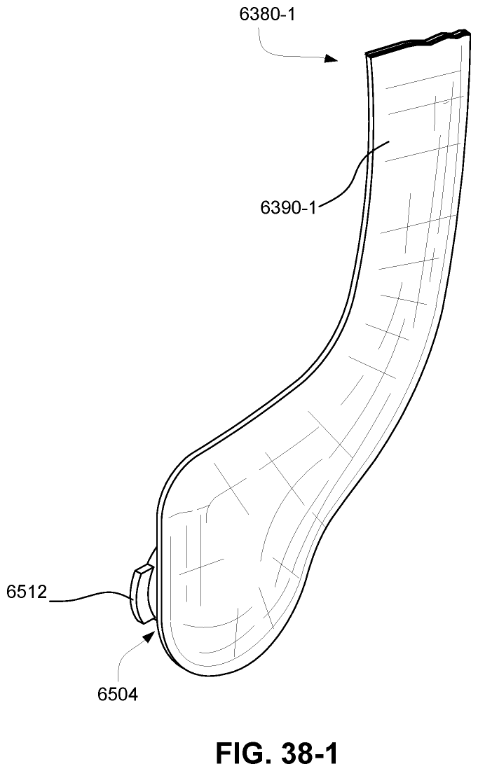

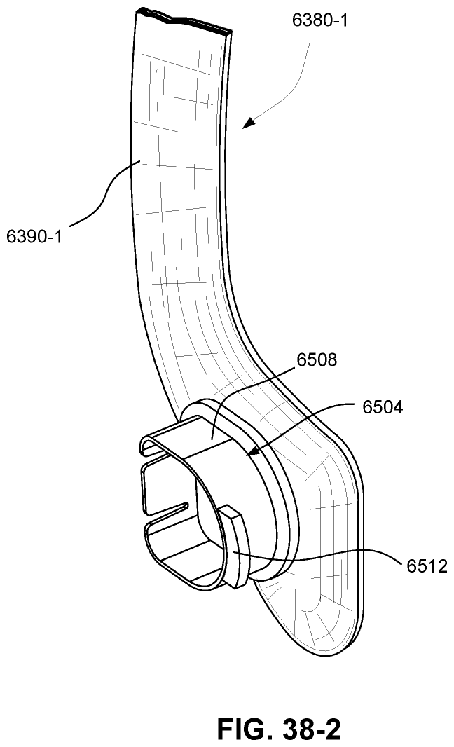

FIG. 38 shows a fifth step in connecting the sleeve of FIG. 23 to the rigidizer arm of FIG. 14 where the sleeve is fully connected to the conduit headgear.

FIG. 38-1 shows a front perspective view of the sleeve of FIG. 23 to the rigidizer arm of FIG. 14.

FIG. 38-2 shows a front perspective view of the sleeve of FIG. 23 to the rigidizer arm of FIG. 14.

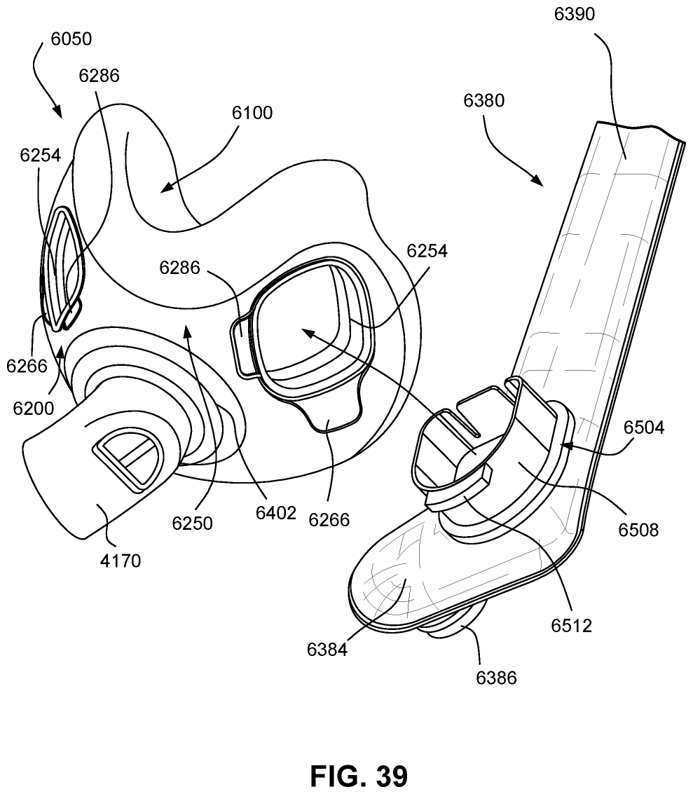

FIG. 39 shows a rear perspective view of the rigidizer arm of FIG. 14 with the sleeve of FIG. 20 to be connected to the cushion of FIG. 7.

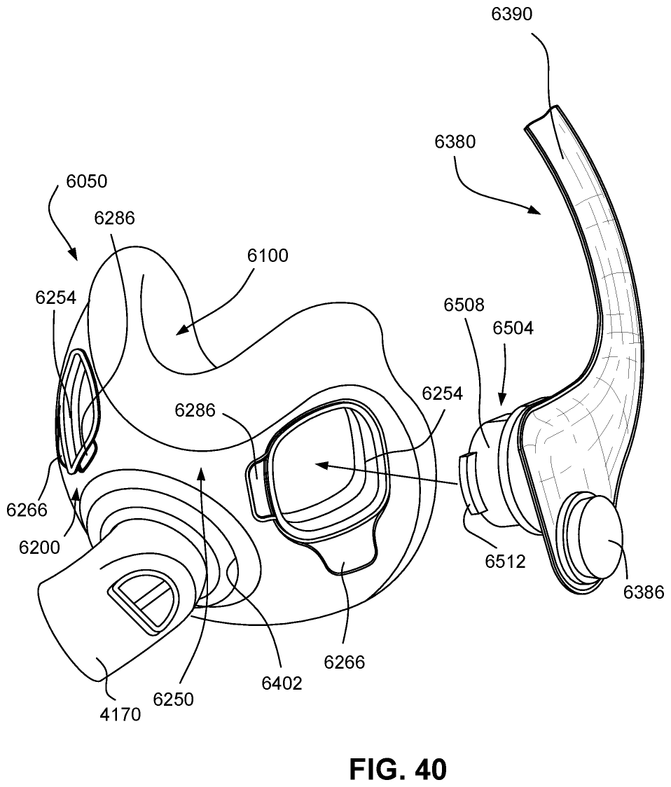

FIG. 40 shows a front perspective view of the rigidizer arm of FIG. 14 with the sleeve of FIG. 20 to be connected to the cushion of FIG. 7.

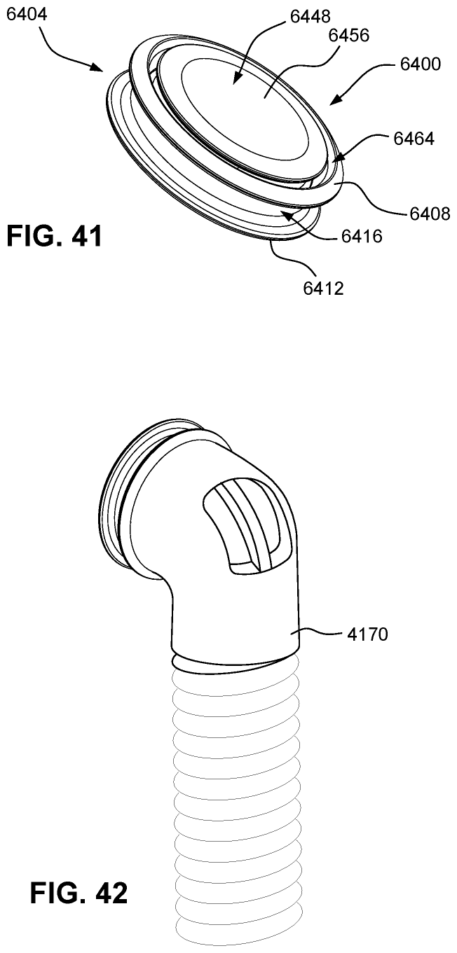

FIG. 41 is a perspective view of a vent removably connectable to the cushion of FIG. 7.

FIG. 42 is a perspective view of an airflow conduit removably connectable to the cushion of FIG. 7.

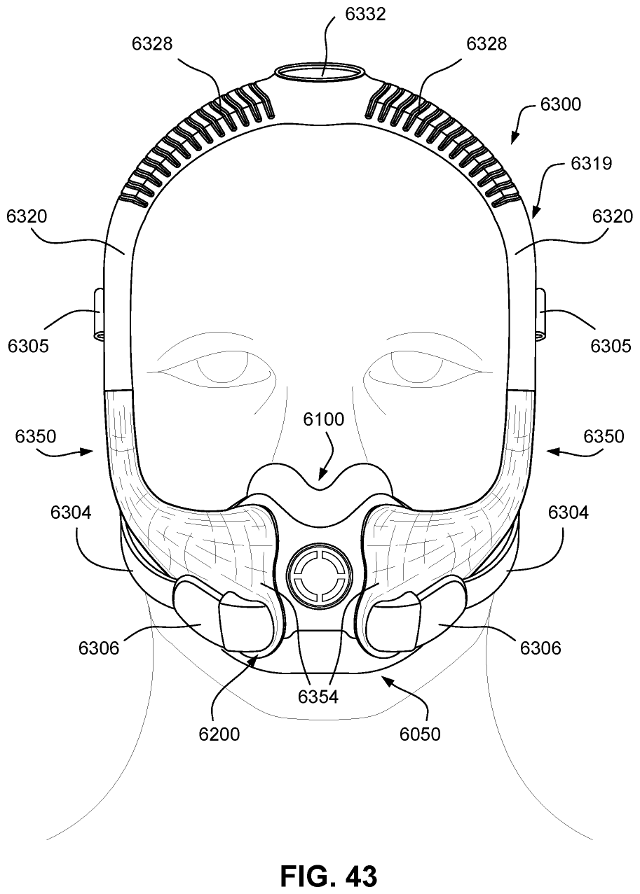

FIG. 43 is a front view of a patient wearing the cushion of FIG. 7 connected to the conduit headgear of FIG. 13, the headgear straps of FIG. 15, and the sleeves of FIG. 17.

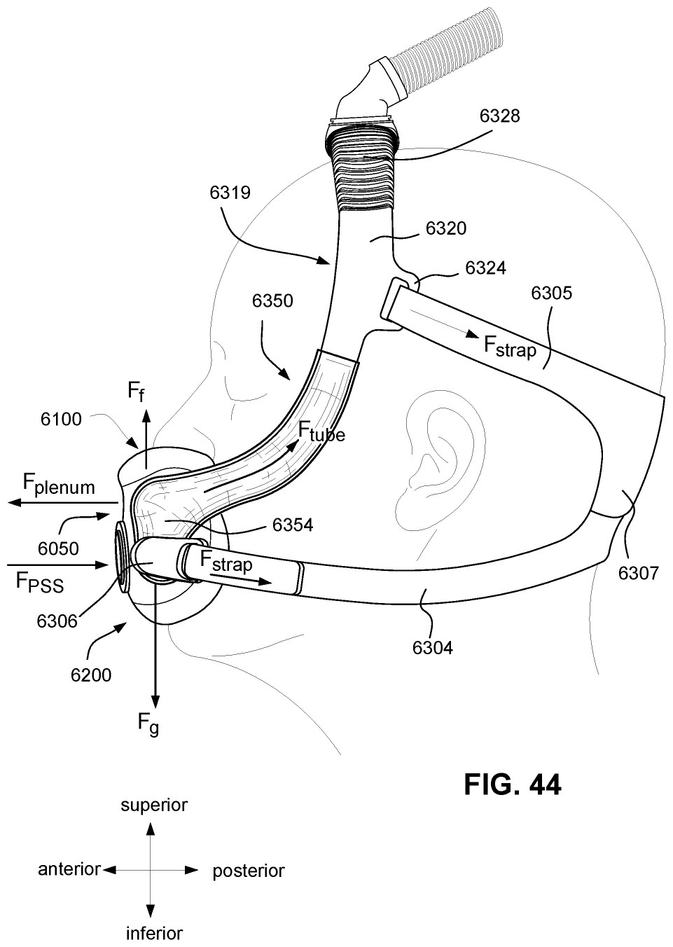

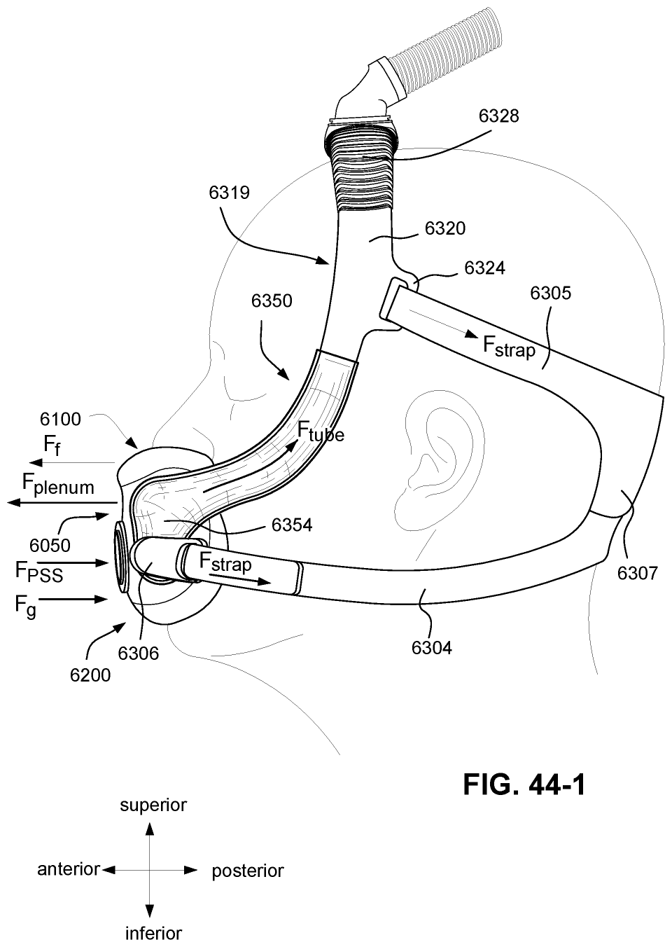

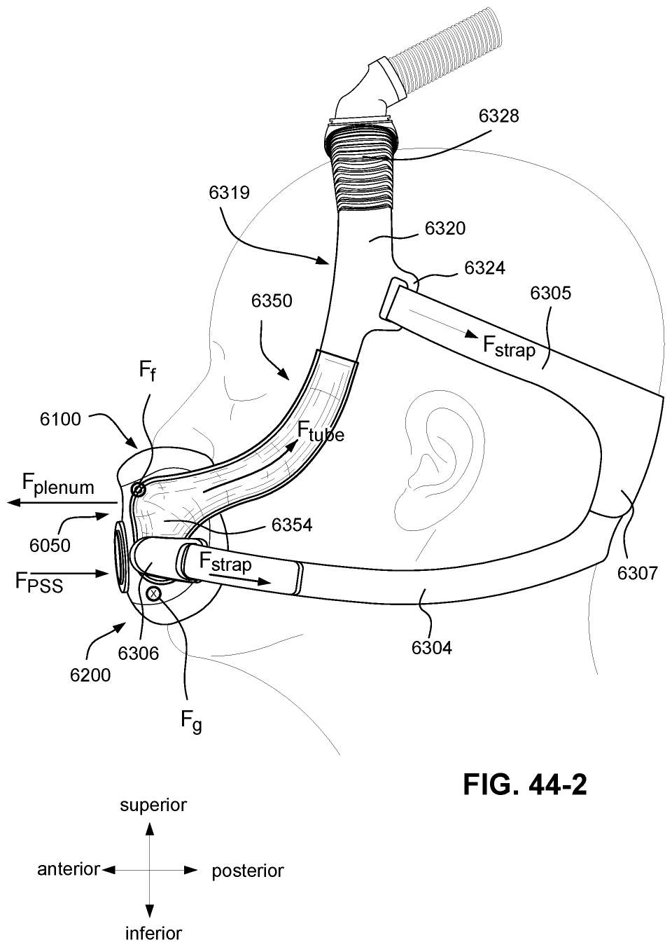

FIG. 44 is a side view of a patient wearing the cushion, conduit headgear, headgear straps, and sleeves of FIG. 43. The patient is oriented in an upright position.

FIG. 44-1 is a side view of a patient wearing the cushion, conduit headgear, headgear straps, and sleeves of FIG. 43. The patient is oriented in a supine position.

FIG. 44-2 is a side view of a patient wearing the cushion, conduit headgear, headgear straps, and sleeves of FIG. 43. The patient is oriented on their side.

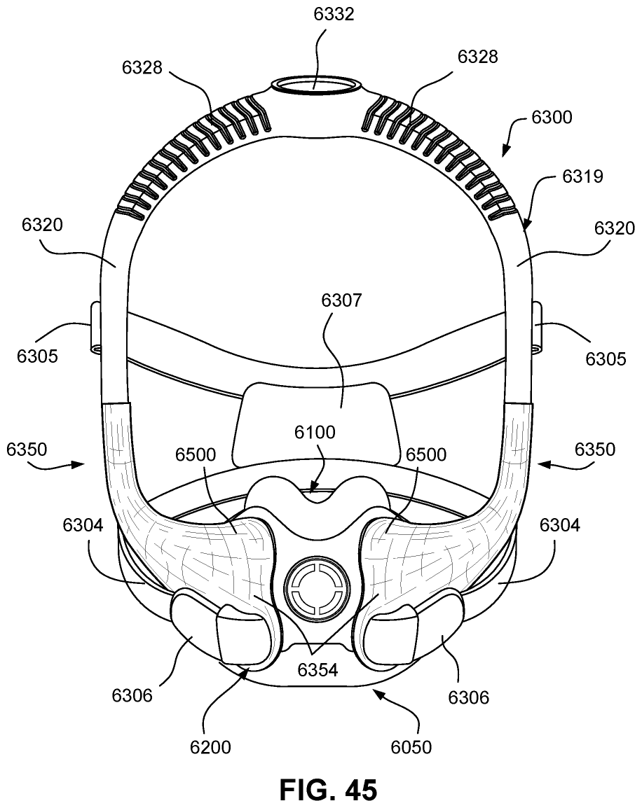

FIG. 45 is a front view of the cushion of FIG. 7 connected to the conduit headgear of FIG. 13, the headgear straps of FIG. 15, and the sleeves of FIG. 17.

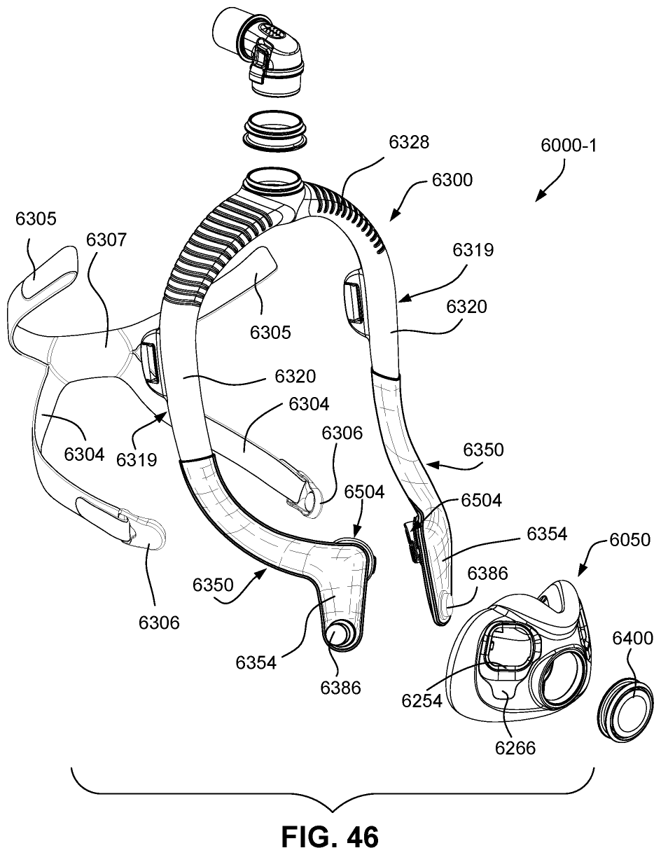

FIG. 46 is an exploded view of the cushion, conduit headgear, headgear straps, and sleeves of FIG. 45.

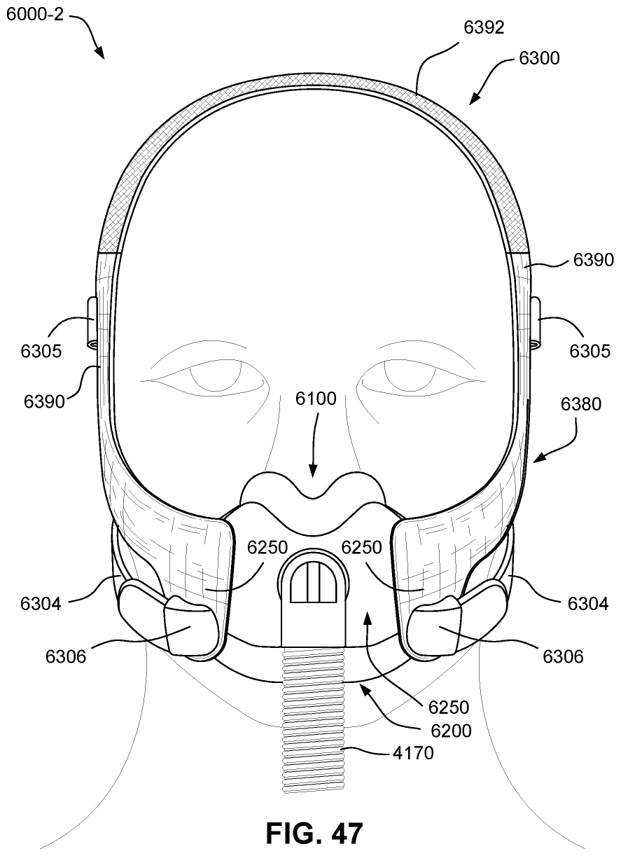

FIG. 47 is a front view of a patient wearing the cushion of FIG. 7 connected to the rigidizer arms of FIG. 14, the headgear straps of FIG. 15, and the sleeve of FIG. 20.

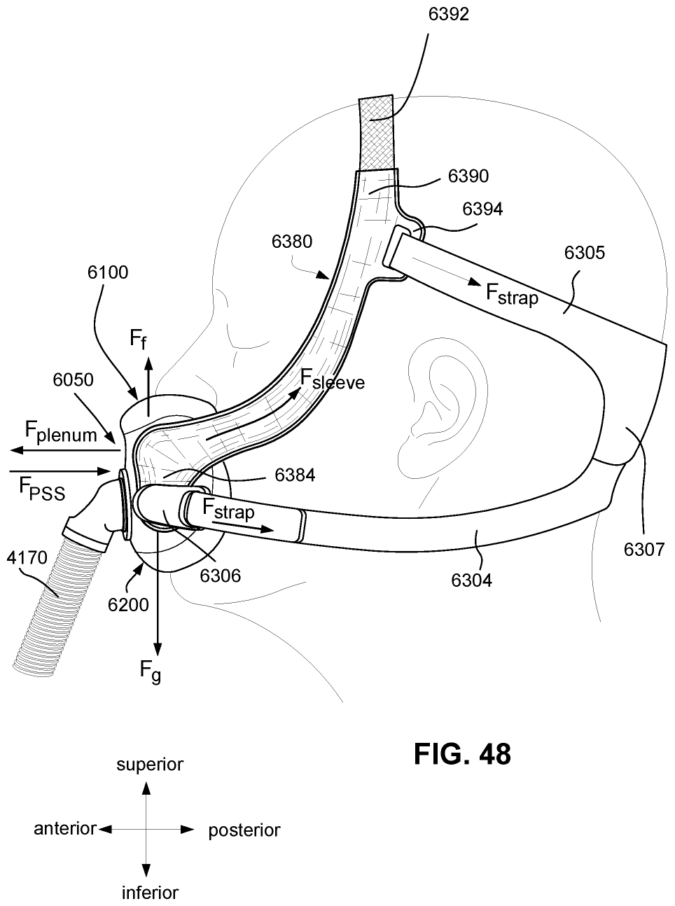

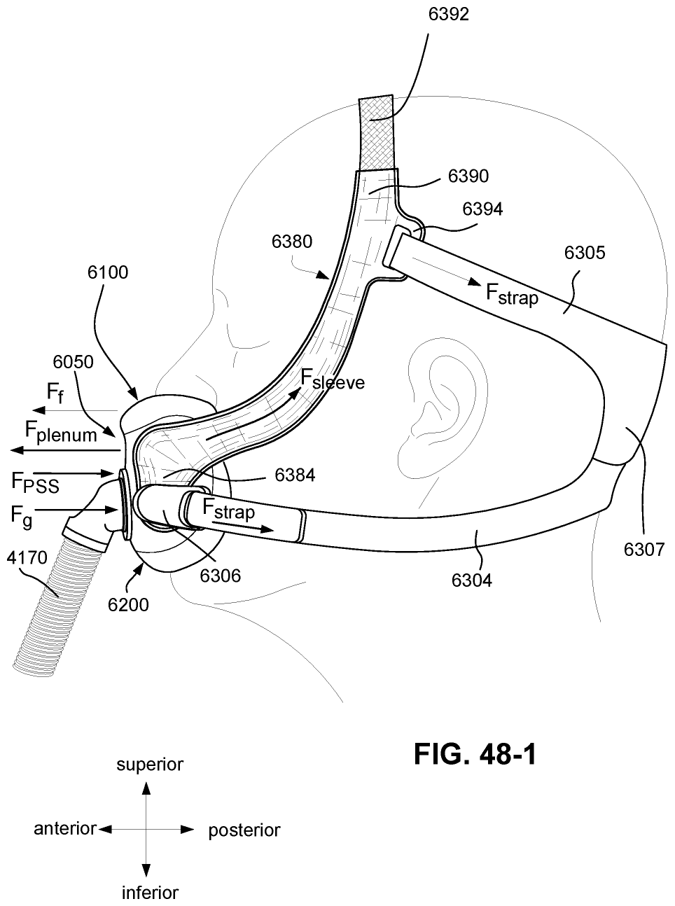

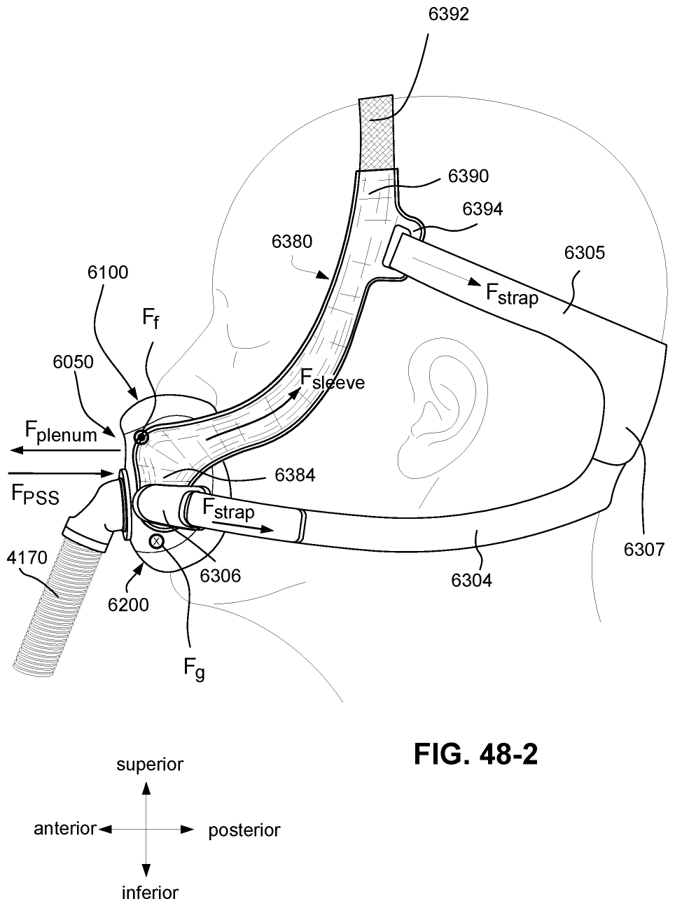

FIG. 48 is a side view of the patient wearing the cushion, conduit headgear, headgear straps, and sleeve of FIG. 47. The patient is oriented in an upright position.

FIG. 48-1 is a side view of a patient wearing the cushion, conduit headgear, headgear straps, and sleeves of FIG. 47. The patient is oriented in a supine position.

FIG. 48-2 is a side view of a patient wearing the cushion, conduit headgear, headgear straps, and sleeves of FIG. 47. The patient is oriented on their side.

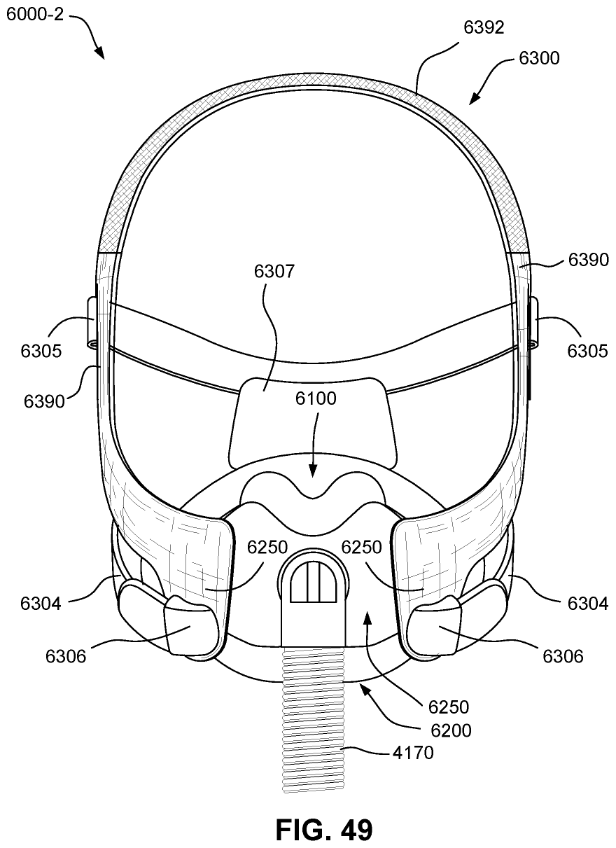

FIG. 49 is a front view of the cushion of FIG. 7 connected to the rigidizer arms of FIG. 14, the headgear straps of FIG. 15, and the sleeve of FIG. 20.

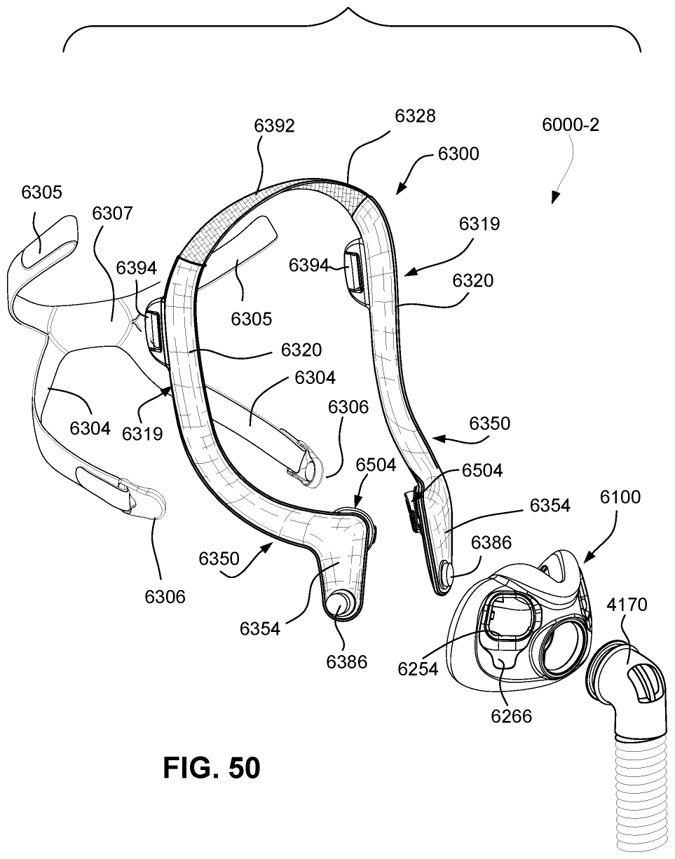

FIG. 50 is an exploded view of the cushion, conduit headgear, headgear straps, and sleeve of FIG. 49.

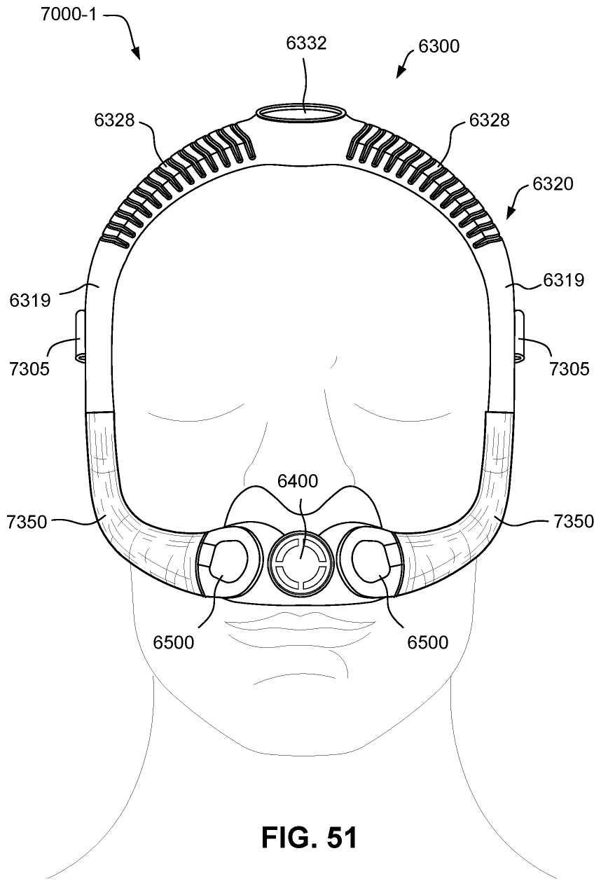

FIG. 51 is a front view of a patient wearing the cushion of FIG. 10 connected to the conduit headgear of FIG. 13, and the headgear straps of FIG. 16.

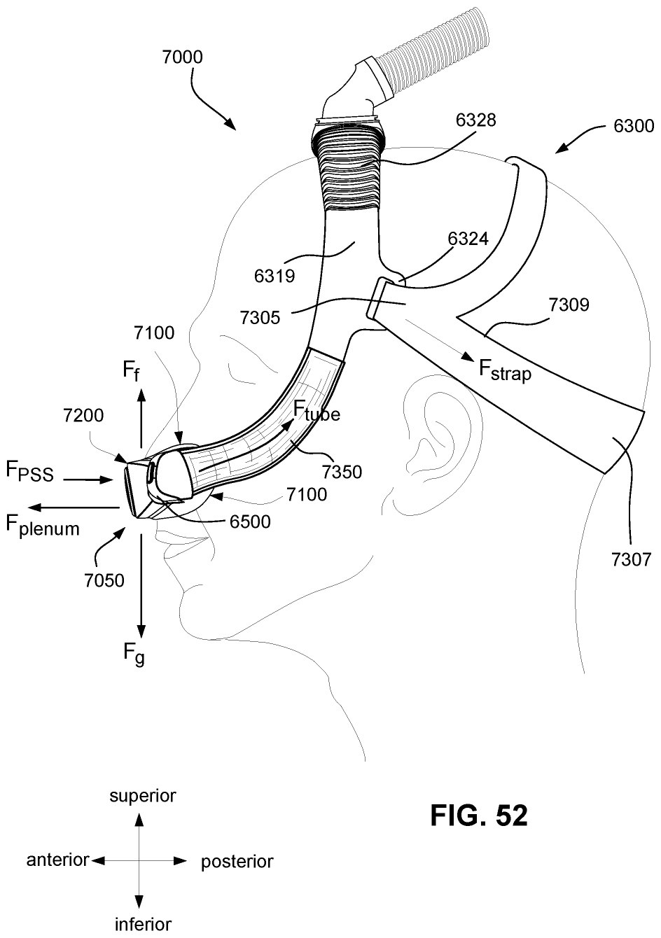

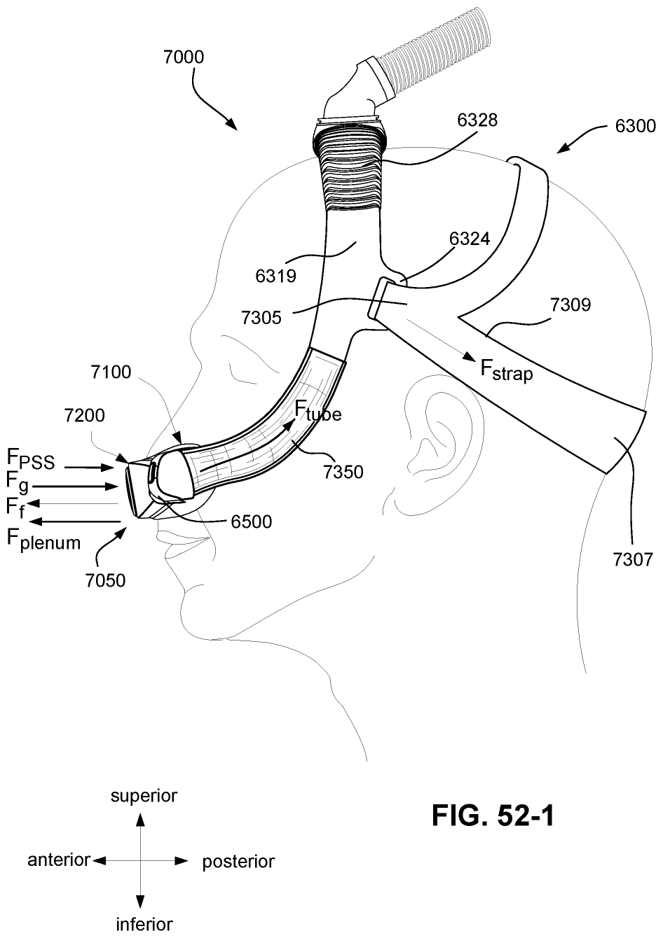

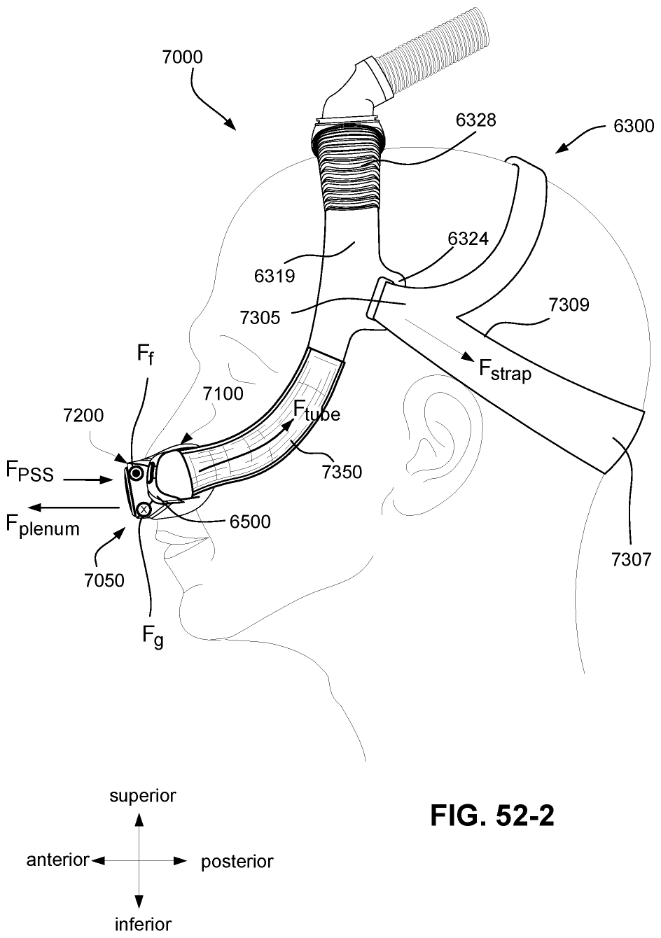

FIG. 52 is a side view of a patient wearing the cushion, conduit headgear, and headgear straps of FIG. 51. The patient is oriented in an upright position.

FIG. 52-1 is a side view of a patient wearing the cushion, conduit headgear, headgear straps, and sleeves of FIG. 51. The patient is oriented in a supine position.

FIG. 52-2 is a side view of a patient wearing the cushion, conduit headgear, headgear straps, and sleeves of FIG. 51. The patient is oriented on their side.

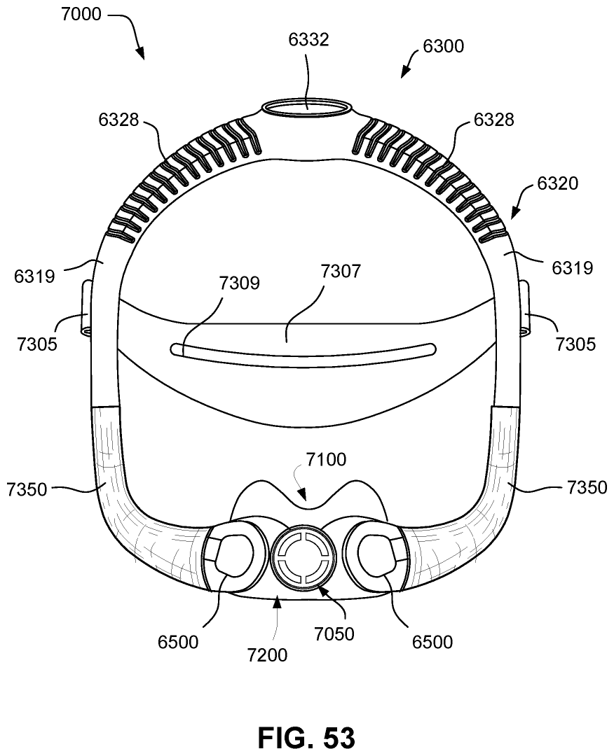

FIG. 53 is a front view of the cushion of FIG. 10 connected to the conduit headgear of FIG. 13, and the headgear straps of FIG. 16.

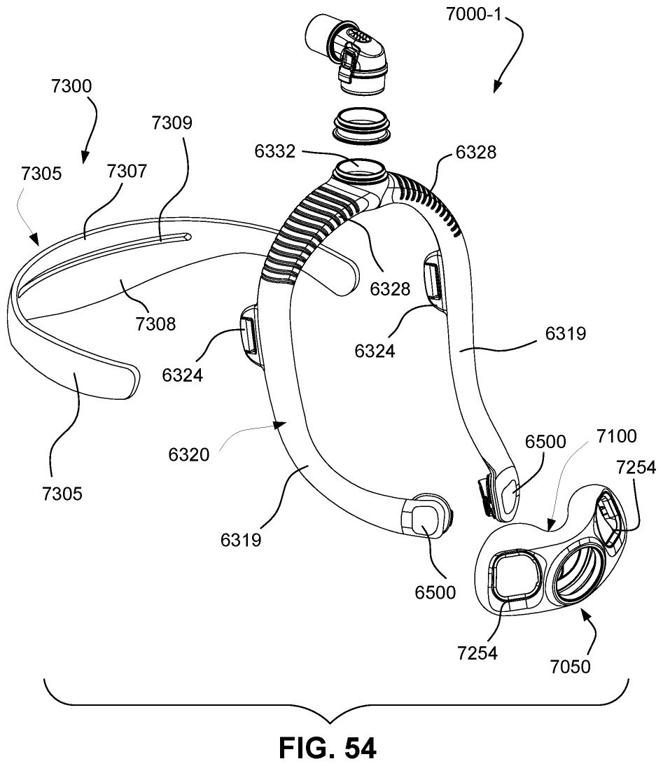

FIG. 54 is an exploded view of the cushion, conduit headgear, and headgear straps of FIG. 53.

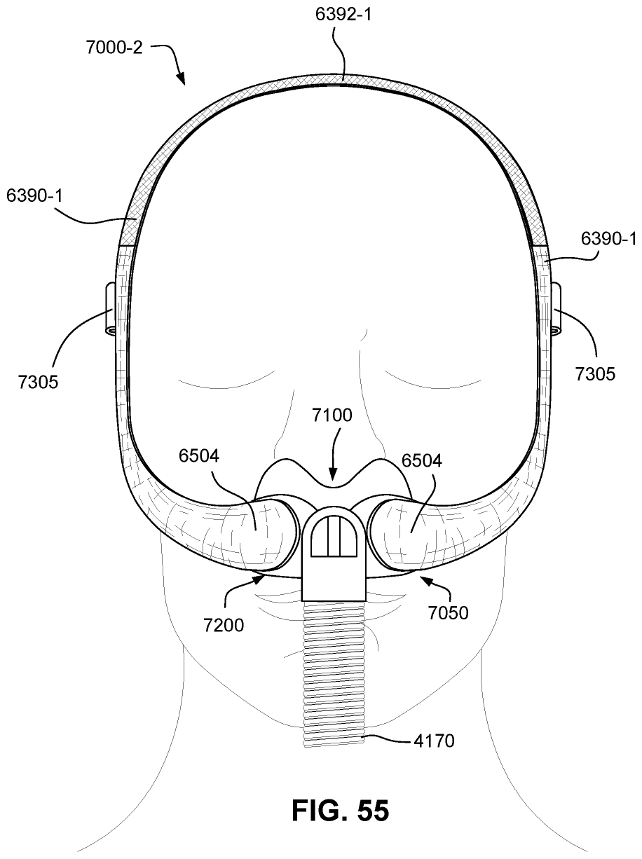

FIG. 55 is a front view of a patient wearing the cushion of FIG. 10 connected to the rigidizer arms of FIG. 14, the headgear straps of FIG. 16, and the sleeve of FIG. 23.

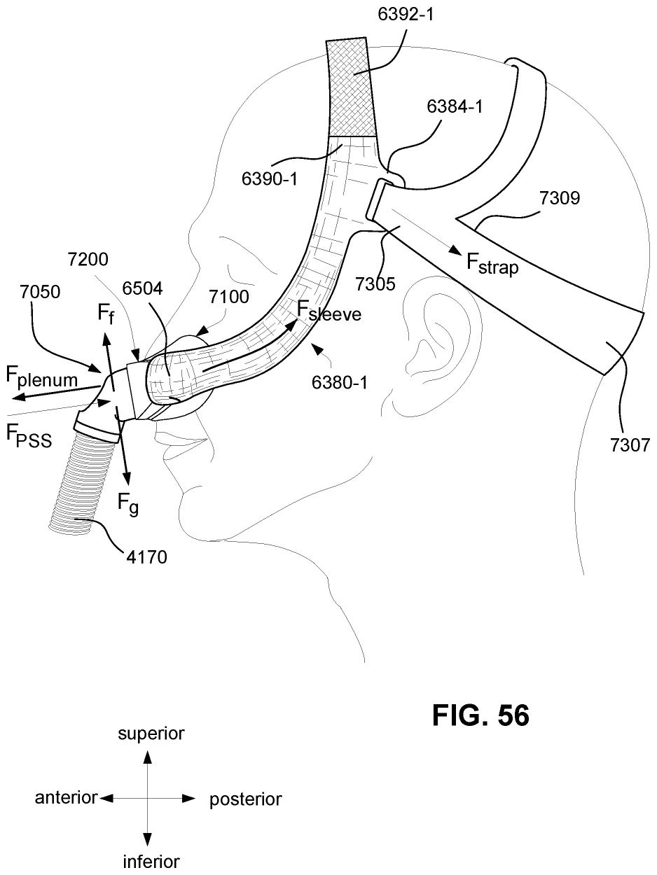

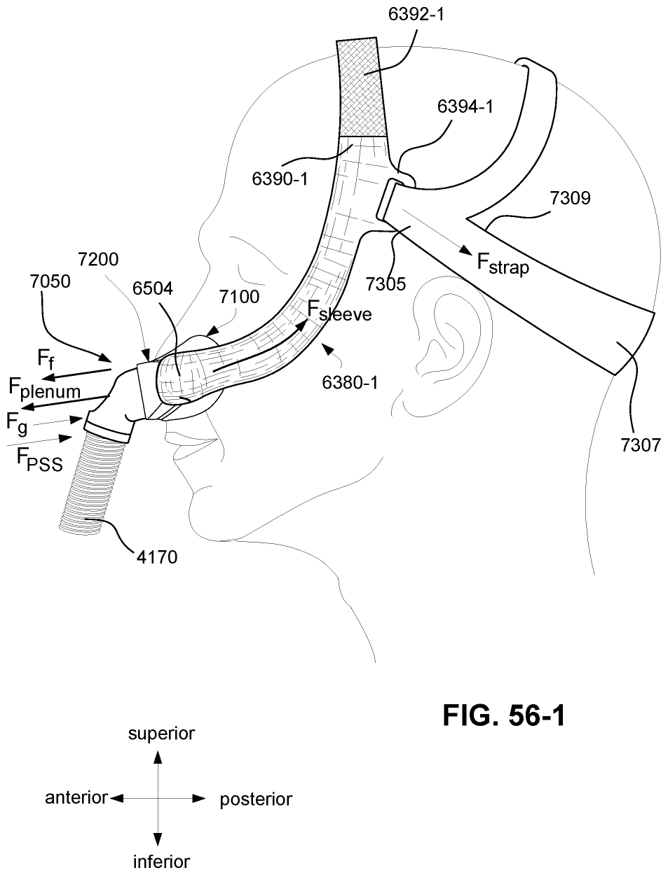

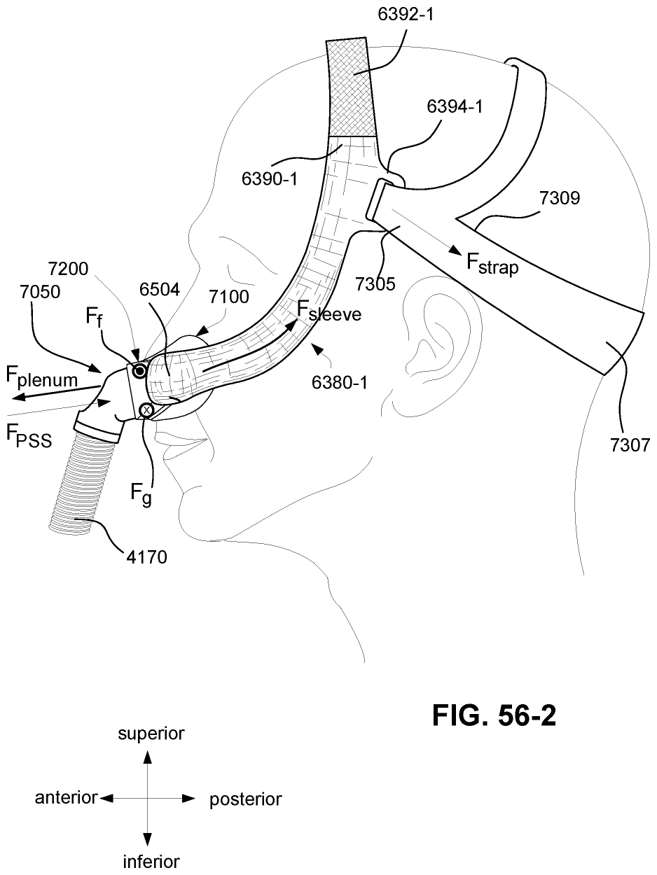

FIG. 56 is a side view of a patient wearing the cushion, rigidizer arms, headgear straps, and sleeve of FIG. 55. The patient is oriented in an upright position.

FIG. 56-1 is a side view of a patient wearing the cushion, conduit headgear, headgear straps, and sleeves of FIG. 55. The patient is oriented in a supine position.

FIG. 56-2 is a side view of a patient wearing the cushion, conduit headgear, headgear straps, and sleeves of FIG. 55. The patient is oriented on their side

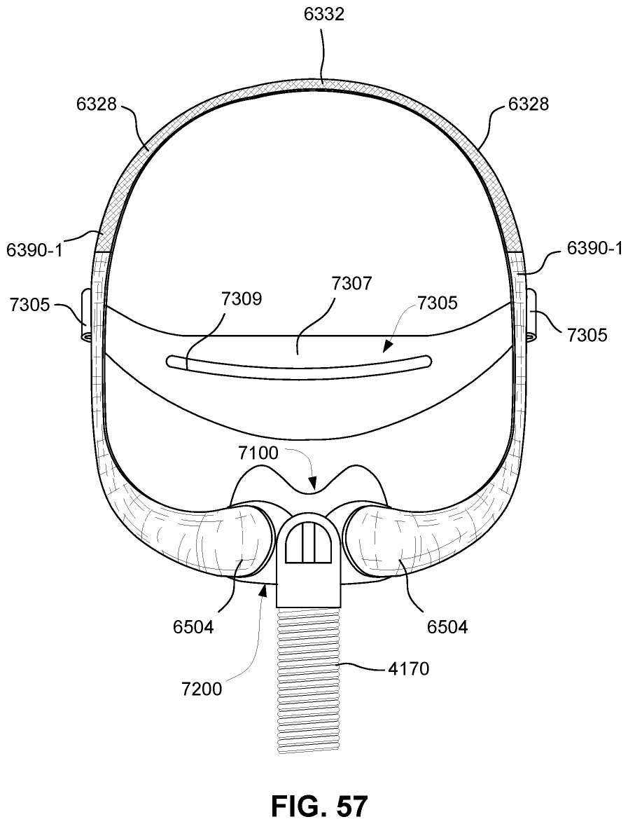

FIG. 57 is a front view of the cushion of FIG. 10 connected to the rigidizer arms of FIG. 14, the headgear straps of FIG. 16, and the sleeve of FIG. 23.

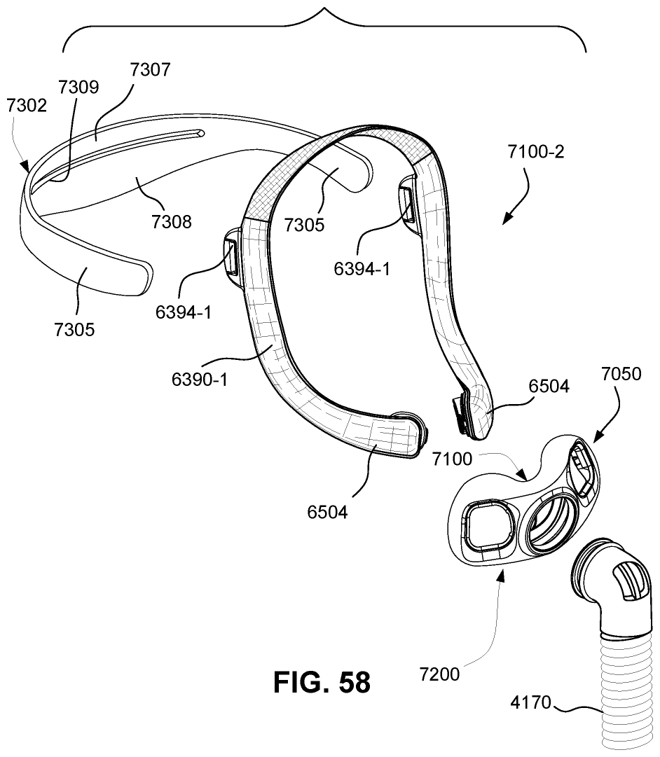

FIG. 58 is an exploded view of the cushion, rigidizer arms, headgear straps, and sleeve of FIG. 57.

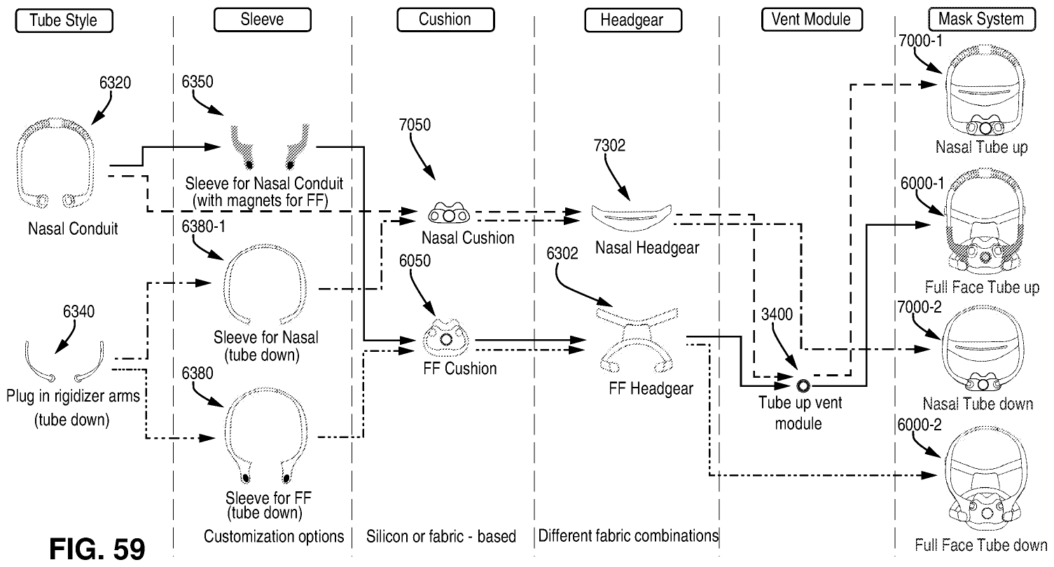

FIG. 59 is a schematic view illustrating the possible combinations of the patient interfaces.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

The patient interfaces 6000-1, 6000-2, 7000-1, and 7000-2 may be similar to the patient interface shown in FIG. 3A, and the features and description of FIG. 3A may be applicable to any of the patient interfaces 6000-1, 6000-2, 7000-1, and 7000-2.

Only some similarities and differences between the different patient interfaces may be described below. Although a certain feature may be described specifically with respect to one example, that description may be applicable to the other examples.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

As is described in greater detail below, in certain forms of the present technology the seal-forming structure 6100 comprises a first seal forming structure 6101 connected to an oral portion 6201 of the plenum chamber 6200 and constructed and arranged to form seal with a region of the patient's face surrounding an entrance to the patient's mouth, and a second seal-forming structure 6102 connected to a nasal portion 6202 of the plenum chamber 6200 constructed and arranged to form a seal with a region of the patient's face surrounding an entrance to the patient's nose (see e.g., FIG. 9). The phrase “connected to” is used herein to refer to portions or components which are formed as a single piece as well as to portions or components which are formed separately and subsequently joined together. In some cases components may be connected by an intermediate component.

In certain forms, the first seal forming structure 6101 seals independently against the patient's face from the second seal forming structure 6102.

In certain forms, the first seal forming structure 6101 and the second seal forming structure 6102 cooperate to form a single common seal against the patient's face.

In one form the target seal-forming region is located on an outside surface of the seal-forming structure 6100.

In certain forms of the present technology, the seal-forming structure 6100 is constructed from a biocompatible material (e.g. silicone rubber, textile, foam, etc.).

A seal-forming structure 6100 in accordance with the present technology may be constructed from a soft, flexible, resilient material (e.g., silicone, textile, foam, etc.). The seal-forming structure 6100 may also be constructed from multiple soft, flexible, resilient materials. For example, a portion of the seal-forming structure 6100 may be silicone and another portion may be textile.

In certain forms of the present technology, a system is provided comprising more than one a seal-forming structure 6100, each being configured to correspond to a different size and/or shape range. For example the system may comprise one form of a seal-forming structure 6100 suitable for a large sized head, but not a small sized head and another suitable for a small sized head, but not a large sized head.

In other forms of the present technology, a seal forming structure 7100 may be connected to the plenum chamber 7200 constructed and arranged to form a seal with a region of the patient's face surrounding an entrance to the patient's nose (see e.g., FIG. 12).

In one form the target seal-forming region is located on an outside surface of the seal-forming structure 7100.

In certain forms of the present technology, the seal-forming structure 7100 is constructed from a biocompatible material (e.g. silicone rubber, textile, foam, etc.).

A seal-forming structure 7100 in accordance with the present technology may be constructed from a soft, flexible, resilient material (e.g., silicone, textile, foam, etc.). The seal-forming structure 7100 may also be constructed from multiple soft, flexible, resilient materials. For example, a portion of the seal-forming structure 7100 may be silicone and another portion may be textile.

In certain forms of the present technology, a system is provided comprising more than one a seal-forming structure 7100, each being configured to correspond to a different size and/or shape range. For example the system may comprise one form of a seal-forming structure 7100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nasal Region

Referring next to FIG. 9, in certain forms of the present technology, the second seal forming structure 6102 comprises a central portion 6110 configured to seal to surfaces of the patient's nose in use. The central portion may seal to an inferior periphery of the patient's nose (e.g. surrounding the patient's nares) and to the patient's lip superior. In examples a portion of the seal-forming structure 6100 may engage the patient's septum. The second seal forming structure 6102 may further comprise lateral portions 6111 on lateral sides of the central portion 6110. In examples, the seal forming structure 6102 may be configured to contact the patient's face below the bridge of the nose or below the pronasale.

In some forms, the central portion 6110 may include nasal openings 6112 that conveys pressurized breathable gas to the patient's nares. There may be one nasal opening 6112 for each nostril (although there may be a single nasal opening). A periphery of the nasal openings 6112 may seal against the patient's nose (e.g., against the patient's alar rims).

With continued reference to FIG. 9, some forms of the central portion 6110 may include a bridge portion 6114 formed between the nasal openings 6112. In use, the bridge portion 6114 may contact the patient's columella and/or subnasale region. The bridge portion 6114 may also contact the patient's nose proximate to the pronasale, but may not contact the ridge of the patient's nose. In other examples, the bridge portion 6114 may not contact the patient's nose beyond or substantially beyond the patient's pronasale in order to avoid contact with the ridge of the patient's nose. The bridge portion 6114 may seal against the patient's nose so that an entire perimeter of each nasal opening 6112 seals against the patient's nose (e.g., in order to limit leaks). The bridge portion 6114 may also limit the patient's nose from extending into the plenum chamber 6200.

In some forms, the bridge portion 6114 may be substantially flat between the nasal openings 6112. This may be as a result of a molding process which gives the bridge portion 6114 its shape. In some examples, the bridge portion 6114 may be in a taut position prior to use by the patient. In other examples, the bridge portion 6114 may be at least partially slack prior to use, and may be under tension as a result of contact with the patient's nose.

With continued reference to FIG. 9, some examples of the bridge portion 6114 may be crimped in order to apply localized tension to the bridge portion 6114 prior to use. For example, the second seal forming structure 6102 may be constructed initially with a relaxed bridge portion 6114, and a crimp may be applied during the manufacturing process in order to increase the tension in the bridge portion 6114. In some forms, the bridge portion 6114 may be crimped in order to allow the second seal forming structure 6102 (or the entire seal-forming structure 6100) to be constructed from a textile material with complex curvatures (e.g., curvatures along multiple, non-parallel axes). The crimped bridge portion 6114 may limit interactions between the various complex curvatures in order to limit the occurrence of leak-forming creases across the surface of the second seal forming structure 6102. The process of crimping is described in International Application No. PCT/AU2021/050344 and U.S. Published Patent Application No. 2020/0246572, each of which are incorporated herein by reference in their entirety.

In some forms, the crimp may be applied to the bridge portion 6114 using an adhesive (e.g., glue). In some forms, the crimp may be applied to the bridge portion 6114 using stitching. In some forms, the crimp may be applied to the bridge portion 6114 using ultrasonic welding. In some forms, the crimp may be applied to the bridge portion 6114 using radio-frequency (RF) welding. In some forms, multiple techniques may be used to form the crimp on the bridge portion 6114.

In some forms, the central portion 6110 may include a positive curvature between the lateral portions 6111. The central portion 6110 may have a substantially small radius of curvature in order to have a tight fit around the patient's nose.

As shown in FIG. 12, the seal-forming structure 7100 may be similarly shaped and/or have a similar structure as compared to the seal-forming structure 6100 described above. Therefore, only some similarities and differences between the seal-forming structures 6100, 7100 will be described below.

The seal-forming structure 7100 may comprise a central portion 7110 configured to seal to surfaces of the patient's nose in use. The central portion may seal to an inferior periphery of the patient's nose (e.g. surrounding the patient's nares) and to the patient's lip superior. In examples a portion of the seal forming structure 7100 may engage the patient's septum. The second seal forming structure 7102 may further comprise lateral portions 7111 on lateral sides of the central portion 7110. In examples, the seal forming structure 7102 may be configured to contact the patient's face below the bridge of the nose or below the pronasale.

In some forms, the central portion 7110 may include nasal openings 7112 that conveys pressurized breathable gas to the patient's nares. There may be one nasal opening 7112 for each nostril (although there may be a single nasal opening). A periphery of the nasal openings 7112 may seal against the patient's nose (e.g., against the patient's alar rims).

With continued reference to FIG. 12, some forms of the central portion 7110 may include a bridge portion 7114 formed between the nasal openings 7112. In use, the bridge portion 7114 may contact the patient's columella and/or subnasale region. Additionally, the bridge portion 7114 may also contact the patient's nose proximate to the pronasale (e.g., inferior to in order to avoid extending beyond the pronasale), but may not contact the ridge of the patient's nose.

As described above, the bridge portion 7114 may be crimped according to the crimping process described in International Application No. PCT/AU2021/050344 and U.S. Published Patent Application No. 2020/0246572.

### 5.3.1.3 Oral Region

As is described above, FIG. 9 shows one form of the non-invasive patient interface 6000 comprises a first seal-forming structure 6101 that forms a seal in use at least partly around the patient's mouth. The first seal forming structure 6101 may form a seal on an inferior region of the patient's face (e.g., the patient's lip inferior and/or the supramenton).

The seal-forming structure 6100 comprises a lip inferior portion 6130 which forms a seal against the lip inferior and/or supramenton of the patient. The lip inferior portion 6130 may be connected to (e.g. contiguous with) a lip superior portion 6131, which forms a seal against the lip superior of the patient. The connection between the lip inferior portion 6130 and the lip superior portion 6131 may form an oral hole 6133.

The seal-forming structure 6100 comprises a relatively low wall thickness (compared to other portions of the interface), for example less than 0.7 mm, at a periphery of the oral hole 6133, the lip inferior portion 6130 of the seal forming structure which lies against the inferior region, and at least the centre of the lip inferior portion 6130. The low wall thickness in these locations assists in achieving an effective, comfortable seal. The seal-forming structure 6100 in these regions is able to readily conform to any complex geometry.

In some forms of the technology the oral hole 6133 is substantially trapezoidal rather than oval or elliptical, in order to more accurately correspond to a shape of the patient's face (e.g., wider beneath the patient's mouth and narrower proximate to the patient's nose). This shape of oral hole may allow the interface 6000 to be particularly compact, and not be substantially wider than a width of the patient's nares. In other examples, the oral hole 6133 may have a rectangular, circular, elliptical, or any other shape.

In some forms, the lip inferior portion 6130 may be continuous with the lip superior portion 6131, which may limit seams or other discontinuities that could otherwise cause discomfort.

As shown in FIGS. 10 to 12, the seal-forming structure 7100 includes only an oral portion and is not intended to seal against a patient's mouth. Thus, when using a patient interface 7000-1, 7000-2 that includes the seal-forming structure 7100, the patient's mouth may be left exposed to the ambient.

### 5.3.1.4 Boundary Between Nasal and Oral Regions

As shown in FIG. 9, in one form of the technology, the boundary between the first sealing forming structure 6101 and the second seal forming structure 6102 forms or comprises a corner or ridge 6120. The corner or ridge 6120 may provide an at least partially sharp boundary between the first and second seal-forming structures 6101, 6102. The corner or ridge 6120 may be rounded, but may include a small radius of curvature.

The corner or ridge 6120 may form a partition between the lip superior portion 6131 of the first seal forming structure 6101 and the central portion 6110 of the second seal forming structure 6102. In use, the corner or ridge 6120 may engage the patient's face above the lip superior and immediately below the nose. The sharp boundary may allow the corner or ridge 6120 to contact the subnasale, but the slight radius of curvature may not significantly decrease patient comfort (e.g., because the corner or ridge 6120 digs into the patient's face).

In some forms, the ridge 6120 forms a relatively sharp angle between the first and second seal forming structures 6101, 6102. The sharp angle reduces the likelihood of creases forming in the first and/or second seal forming structures 6101, 6102 on or adjacent to the corner or ridge 6120 when the mask is donned and therapy is applied. Some oro-nasal patient interfaces which do not use such a structure may require a very thin, rounded formation in this area which may be less resistant to creasing. By contrast, the corner or ridge 6120 may be stiffer, and may hold its shape better, than such interfaces and may therefore seal better against the concavities and creases present around the patient's nose. This effect may be enhanced in embodiments which are provided with support portions, which resist or oppose compression of this region.

In some forms of the technology the radius of the corner or ridge 6120 may be less than 2 mm, for example around 1.75 mm. In one form of the technology the radius may vary from approximately 1.75 mm in the centre of the ridge to approximately 0.75 mm at the lateral portions.

The angle formed by the first and second sealing structures may be about 20 degrees to about 90 degrees, for example about 36 degrees.

In some forms of the technology, the corner or ridge 6120 may extend across substantially an entire boundary 6103 between the first seal forming structure 6101 and the second seal forming structure 6102. In embodiments the corner or ridge 6120 may engage the patient's face at least approximate the entrances to the nares, for example where the ala meets the face above the lip superior.

In other forms, a boundary between the first and second seal forming structures 6101, 6102 may include a smooth or substantially smooth transition. The smooth surface between along the smooth boundary may promote patient comfort because sharp surfaces are reduced.

As described above, the seal-forming structure 7100 may not include a boundary region because the seal-forming structure 7100 only seals against a patient's nose, and does not seal around a patient's mouth.

### 5.3.1.5 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

As shown in FIGS. 43 and 47, the seal-forming structure 6100 may contact the patient's face in order to minimize contact with the nose bridge region or on the nose-ridge region of the patient's face. In some example, the seal-forming structure 6100 may be situated so that the patient's pronasale is exposed in use. This may increase patient comfort because sensitive regions along the nose bridge region or on the nose-ridge region of the patient's face are not in a pressurized environment.

As shown in FIGS. 51 and 55, the seal-forming structure 7100 may similarly minimize contact with the nose bridge region or the nose-ridge region of the patient's face.

In other examples (not shown), the seal-forming structure 6100 and/or the seal-forming structure 7100 may be structured in order to contact and seal against the patient's nasal ridge.

### 5.3.1.6 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

As described above, the upper lip region may assist in forming a seal at least partially around the patient's nares, and at least partially around the patient's mouth (e.g., in a full-face patient interface). The upper lip region may also assist in forming a seal around only the patient's nares (e.g., in a nasal-only patient interface).

### 5.3.1.7 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.8 Forehead Region

In one form, the seal-forming structure that forms a seal in use on a forehead region of the patient's face. In such a form, the plenum chamber may cover the eyes in use.

### 5.3.1.9 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.2 Plenum Chamber

As shown in FIG. 3A, the plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 (or at least a portion of the plenum chamber 3200) and the seal-forming structure 3100 (or at least a portion of the seal-forming structure 3100) are formed from a single homogeneous piece of material (e.g., molded silicone, woven textile, etc.). A combination of the seal-forming structure 3100 and the plenum chamber 3200 may be considered a cushion.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 5.3.2.1 Flexible Shell

In some forms of the technology, the plenum chamber 6200 may include a shell 6250, which may be constructed from a rigid material such as polycarbonate. The rigid material may provide support to the seal-forming structure 6100.

As shown in FIGS. 7 to 12, some forms of the shell 6250, or portions of the shell 6250, of other forms of the technology may be somewhat flexible (e.g., constructed from a soft, flexible, resilient material like silicone, textile, foam, etc.). For example, in examples the shell 6250 may be formed from a material which has a Young's modulus of 0.4 GPa or lower, for example foam. In some forms of the technology the shell 6250 may be made from a material having Young's modulus of 0.1 GPa or lower, for example rubber. In other forms of the technology the shell 6250 may be made from a material having a Young's modulus of 0.7 MPa or less, for example between 0.7 MPa and 0.3 MPa. An example of such a material is silicone.

In certain forms, the shell 6250 and one or both of the first and second seal forming structures 6101, 6102 may be formed from the same material (e.g., silicone, textile, etc.). The shell 6250 and the seal-forming structures 6101, 6102 may be removable from one another or may be a single homogeneous piece of material.

In some forms of the technology, the shell 6250 may be constructed substantially entirely from a flexible material, which may provide the shell 6250 with the greatest freedom of movement (i.e., substantially no rigid and/or thickened portions that limit bending). The shell 6250 may necessitate that one or more components are added to provide a required stiffness in one or more areas or regions of the shell 6250 (e.g., in order to limit creasing of the seal-forming structure 6100 proximate to the nasal alar region). For example, one or more of a vent module; a connection port; a headgear connector; a headgear connector connected to a rigidizing arm and a rigidizing member may be connected to the shell 6250 in such a way as to increase the stiffness of the plenum chamber 6200 in the area adjacent the component, for example as described further below. In some forms of the technology such components may be releasably connectable to the flexible shell 6250.

Additionally or alternatively one more components may be permanently connected to the shell 6250, for example by bonding and/or overmolding. The rigidizing member may also serve to increase the stiffness and/or support the shape of the seal-forming structure 6100. In certain forms of the present technology, the permanently connected rigidizing members may be dedicated stiffening members or rigidizing members (e.g., with no other function).

In some forms of the technology the shell 6250 may be generally flexible but may comprise stiffening portions having greater thickness than immediately adjacent portions of the shell 6250. Such stiffening portions may be configured as ribs or bands, for example extending laterally across the shell and/or extending in a superior-inferior direction, although many other configurations are possible. In some forms the shell may comprise a substantially rigid portion, for example manufactured from polycarbonate, as well as a somewhat flexible portion.

In some forms of the technology it may be preferable for a central portion 6251 of the anterior side of the oral portion 6201 of the plenum chamber to have a greater stiffness than the remainder of the plenum chamber 6200. In some forms of the technology the area of increased stiffness may be immediately inferior to the nasal portion 6202, and/or immediately superior to the oral portion 6201. In one form of the technology, a portion of, or the entirety of, the first anterior wall portion 6240 may be an area of increased stiffness, rather than an area of increased flexibility. Providing increased stiffness in one or more of these areas may provide shape stability and may limit the extent to which the shell 6250 deforms as a result of headgear forces. Excessive deformation may result in the second seal forming structure 6102 occluding the nares. Avoiding such deformation may be particularly advantageous to patients with relatively wide noses, and may be less important, or in some cases undesirable, for patients with narrow noses. In addition, the areas of increased stiffness described may assist in reducing torsional deformation of the interface which may otherwise result in one side of the second seal forming structure 6102 losing contact with the patient's nose, thereby creating a leak path.

### 5.3.2.1.1 Multiple Opening

As shown in FIGS. 7 to 12, the plenum chamber 6200, 7200 may be formed as part of a multi-opening cushion 6050, 7050. In the illustrated examples, the cushions 6050, 7050 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 6200, 7200. For example, depending on patient need and/or patient comfort, the patient may use a given cushion 6050, 7050 in a “tube-up” configuration (e.g., using conduit headgear—described below) or a “tube-down” configuration (e.g., using a single conduit in front of the patient's face).

### 5.3.2.1.1.1 Full-Face Cushion

As shown in FIGS. 7 to 9, the plenum chamber 6200 may be included in a full-face patient interface 6000 (e.g., a full-face mask, an ultra-compact full-face mask, etc.), which includes the first and second seal forming structures 6101, 6102 described above.

As shown in FIGS. 8 and 9, the plenum chamber 6200 includes a pair of plenum chamber inlet ports 6254, which may be used to convey gas into and/or out of the plenum chamber 6200. The plenum chamber inlet ports 6254 may be disposed on opposite sides (e.g., left and right sides) of the plenum chamber 6200.

In some forms, each plenum chamber inlet port 6254 includes a partially rectangular shape. For example, the plenum chamber inlet port 6254 may include at least one substantially straight side. The corners between the different sides may also be rounded. In the illustrated example, each plenum chamber inlet port 6254 may include one curved side 6255. The curved side 6255 may be disposed proximate to a center of the plenum chamber 6200, and may extend generally in the superior-inferior direction. The remainder of the illustrated sides of the plenum chamber inlet port 6254 may be substantially straight sides, although any number of the sides may be curved.

In other examples, the plenum chamber inlet ports 6254 may include an elliptical shape, a circular shape, or any similar shape. For example, the plenum chamber inlet port 6254 may include a rounded shape. In other forms, the plenum chamber inlet port 6254 may be symmetric about only a single axis.

As shown in FIG. 8, each plenum chamber inlet port 6254 may be symmetric about only a single axis. For example, an axis bisecting the curved side 6255 of each respective plenum chamber inlet port 6254 may form an axis of symmetry. As described below, this may help prevent improper connection of a conduit. Additionally, in other forms, the plenum chamber inlet ports 6254 may not be uniform with one another and/or the plenum chamber inlet ports 6254 may not have any axes of symmetry.

In some forms, the plenum chamber inlet port 6254 may be disposed on the oral portion 6201 of the plenum chamber 6200. In the illustrated example, each plenum chamber inlet port 6254 may extend proximate to the transition between the oral portion 6201 and the nasal portion 6202 of the plenum chamber 6200. The plenum chamber inlet port 6254 may be positioned at least partially superior to the patient's mouth (e.g., as determined when a patient is in an upright position, an axis through the plenum chamber inlet port 6254 (e.g., perpendicular to the plenum chamber inlet port 6254) may be aligned with the patient's mouth while the patient interface 6000 is in use.

In some forms, the oral portion 6201 of the plenum chamber 6200 may have a substantially negatively domed curvature (e.g., when facing the anterior surface). The plenum chamber inlet ports 6254 may be positioned on the curved surface of the central portion 6251 of the plenum chamber 6200, and may be on either side of an apex of the curvature. The plenum chamber inlet ports 6254 may be aligned so that a single axis may pass through both plenum chamber inlet ports 6254. The axis may be substantially perpendicular with the patient's sagittal plane.

In some forms, the plenum chamber 6200 may also include at least one vent opening 6402 (see e.g., FIG. 7). The vent opening 6402 may be disposed in a center of the plenum chamber 6200. For example, the vent opening 6402 may be disposed between the plenum chamber inlet ports 6254.

In some forms, the vent opening 6402 may be disposed inferior to at least a portion of each plenum chamber inlet port 6254. For example, the vent opening may be disposed proximate to an inferior-most portion of the plenum chamber 6200.

In certain forms, the vent opening 6402 may have a rounded perimeter. For example, the vent opening 6402 may have a circular perimeter. In other examples, the vent opening 6402 may have an elliptical perimeter, or it may have a perimeter formed from a different polygonal shape (e.g., triangle, rectangle, etc.). These polygonal shapes may have angled corners, or they may have rounded corners.

In some forms, the vent opening 6402 may be aligned with the patient's mouth while the patient interface is in use. In other words, the vent opening 6402 may be disposed directly in front of the patient's mouth when he is wearing the patient interface 6000. Air exhaled by the patient (e.g., through his mouth) may travel directly toward the vent opening 6402.

In some forms, the material surrounding the vent opening 6402 may be substantially flush with the central portion 6251 of the plenum chamber 6200. This may help to maintain a substantially small device footprint. For example, material surrounding the central portion 6251 may not extend substantially far from the patient's face and obstruct the patient's view while the patient interface 6000 is in use. In other examples, material surrounding the vent opening 6402 may protrude from the central portion 6251.

As shown in FIGS. 7 and 8, certain forms of the plenum chamber 6200 may include a groove 6266 that may be disposed along the sides of the plenum chamber 6200.

In some forms, the plenum chamber 6200 may include a pair of grooves 6266. Each groove 6266 may be disposed proximate to one of the plenum chamber inlet ports 6254. Each groove 6266 may form a partially recessed surface.

In certain forms, an area of each groove 6266 may be larger than an area of each plenum chamber inlet port 6254. Additionally, the shape of each groove 6266 may not correspond to the shape of each plenum chamber inlet port 6254 (although they may). For example, each plenum chamber inlet port 6254 may be proximate a superior end of the respective groove 6266. The groove 6266 may extend toward an inferior portion of the plenum chamber 6200 beyond the perimeter of the respective plenum chamber inlet port 6254. Each groove 6266 may have substantially the same depth throughout (although the depth may be varied).

### 5.3.2.1.1.2 Nasal Cushion

As shown in FIGS. 10 to 12, the plenum chamber 7200 may be included in a nasal patient interface 7000, which seals only in or around the patient's nares and leaves the patient's mouth exposed to the ambient. As described above, the nasal patient interface 7000 includes only a single seal-forming structure 7100 (e.g., similar to the second seal forming structure 6102), and does not include separate the first and second seal forming structures like in the full-face patient interface 6000.

The plenum chamber 7200 of the nasal patient interface 7000 may be similar to the plenum chamber 6200 of the full-face patient interface 6000. Only some similarities and differences between the plenum chambers 6200, 7200 may be described below.

As shown in FIGS. 11 and 12, the plenum chamber 7200 includes a pair of plenum chamber inlet ports 7254, which may be used to convey gas into and/or out of the plenum chamber 7200. The plenum chamber inlet ports 7254 may be disposed on opposite sides (e.g., left and right sides) of the plenum chamber 7200.

In the illustrated example, the shape of the plenum chamber inlet ports 7254 may be substantially the same as the shape of the plenum chamber inlet ports 6254 described above. As described below, this may allow a single connector to be interchangeably connected to the plenum chamber inlet ports 6254, 7254 on either plenum chamber 6200, 7200.

In some forms, the plenum chamber 7200 may also include at least one vent opening 7402 (see e.g., FIG. 10). The vent opening 7402 may be disposed in a center of the plenum chamber 7200. For example, the vent opening 7402 may be disposed between the plenum chamber inlet ports 7254.

In the illustrated example, the plenum chamber inlet ports 7254 and the vent opening 7402 may be aligned along a single axis on the plenum chamber 7200. For example, the plenum chamber inlet ports 7254 may sit at a similar position relative to the patient's face as the plenum chamber inlet ports 6254. However, because the overall plenum chamber 7200 is smaller than the plenum chamber 6200 (i.e., because the plenum chamber 7200 does not receive the patient's mouth), the vent opening 7402 cannot be positioned in line with the patient's mouth, as in the plenum chamber 6200. The vent opening 7402 of the plenum chamber 7200 therefore must be positioned higher on the patient's face as a result of the plenum chamber 7200 only including a nasal portion.

In some forms, the vent opening 7402 of the plenum chamber 7200 may be aligned with the patient's lip superior when the patient is wearing the cushion 7050.

In some forms, the material surrounding the vent opening 7402 may be substantially flush with the central portion 7251 of the plenum chamber 7200. This may help to maintain a substantially small device footprint. For example, material surrounding the central portion 7251 may not extend substantially far from the patient's face and obstruct the patient's view while the patient interface 7000 is in use. In other examples, material surrounding the vent opening 7402 may protrude from the central portion 7251.

As shown in FIGS. 10 and 11, certain forms of the plenum chamber 7200 may include a groove 7266 that may be disposed along the sides of the plenum chamber 7200.

In some forms, the plenum chamber 7200 may include a pair of grooves 7266. Each groove 7266 may be disposed proximate to one of the plenum chamber inlet ports 7254. Each groove 7266 may form a partially recessed surface.

Unlike the grooves 6266 described above, the grooves 7266 may be substantially the same size as the plenum chamber inlet ports 7254. In other words, the grooves 7266 may not extend further in an inferior direction than in the lateral directions.

### 5.3.3 Positioning and Stabilising Structure

As shown in FIG. 3A, the seal-forming structure 3100 of the patient interface 3000 of the present technology may be held in sealing position in use by the positioning and stabilising structure 3300.

In one form the positioning and stabilising structure 3300 provides a positioning and stabilizing structure force F_{PSS} at least sufficient to overcome the effect of the positive pressure in the plenum chamber 3200 to lift off the face (i.e., Fₚₗₑₙᵤₘ).

In one form the positioning and stabilising structure 3300 provides a retention force to overcome the effect of the gravitational force on the patient interface 3000.

With continued reference to FIG. 3A, the positioning and stabilizing structure 3300 provides a positioning and stabilizing structure force F_{PSS} (or positioning and stabilizing force F_{PSS}) that assists in maintaining the plenum chamber 3200 in the sealing position on the patient's face. The positioning and stabilizing force F_{PSS} may be the resultant force from the various force vectors of the different elements of the positioning and stabilizing structure 3300. For example, headgear straps may individually provide a strap force Fₛₜᵣₐₚ in order to hold the seal-forming structure 3100 against the patient's face. The force Fₛₜᵣₐₚ may also be directed at least partially in the superior direction in order to overcome the gravitational force F_{g}. The gravitational force F_{g} may be specifically shown for the seal-forming structure 3100 and the plenum chamber 3200, but gravity would act on the entirely of the patient interface 3000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A), the frictional force F_{f} would act in the superior direction (e.g., against a patient's face). For example, the patient may experience the frictional force F_{f} against his lip superior (and/or other surfaces of the patient's face in contact with the seal-forming structure 3100) in order to oppose the motion in the inferior direction (which may help to stabilizing the cushion 6050 in place). Although the frictional force F_{f} is shown specifically opposing the gravitational force F_{g} of the seal-forming structure 3100 and the plenum chamber 3200, components of an overall frictional force (not shown) would also oppose the gravitational force F_{g} associated with the positioning and stabilizing structure 3300 and any other portions of the patient interface 3000. A force of friction can act along any place where the patient interface 3000 contacts the patient's skin (or hair). The frictional force F_{f} extends in the opposite direction of the gravitational force F_{g} and along the patient's skin (or hair).

In some forms, the sum of the various forces may equal zero so that the patient interface 3000 is at equilibrium (e.g., not moving along the patient's face while in use). Specifically, the gravitational force F_{g} and the blowout force Fₚₗₑₙᵤₘ tend to move the seal-forming structure 3100 away from the desired sealing position. The positioning and stabilizing force F_{PSS} is applied in order to counteract the gravitational force F_{g} and the blowout force Fₚₗₑₙᵤₘ (as well as any frictional forces F_{f}) and keep the seal-forming structure 3100 properly situated. Although the positioning and stabilizing force F_{PSS} may exceed the sum of the other forces and still maintain the seal-forming structure 3100 in an appropriate sealing position, patient comfort may be sacrificed. Maximum patient comfort may be achieved when the net force on the patient interface 3000 is zero and the positioning and stabilizing force F_{PSS} is exactly strong enough to achieve this. As described below, various positions of the patient's head while using the patient interface 3000 may determine the positioning and stabilizing force F_{PSS} necessary to achieve equilibrium.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

In some forms, one version of style of a positioning and stabilising structure may be interchanged with multiple styles or versions of a cushion having a seal-forming structure and a plenum chamber. For example, the previous sections describe two different cushions; the full-face cushion 6050 and the nasal cushion 7050. A single positioning and stabilising structure may be used interchangeably with both versions of cushions 6050, 7050 (or other versions not explicitly described here).

The interchangeability of positioning and stabilising structure between different styles or versions of cushions may simplify manufacturing and/or may allow a patient to easily switch between different cushions without needed to acquire a fully new assembly.

### 5.3.3.1 Conduit Headgear

Conduits, like headgear straps, may provide a force that contributes to the positioning and stabilizing force F_{PSS}. For example, each conduit may provide a force F_{conduit} directed in the posterior and respective lateral direction in order to hold the seal-forming structure 3100 against the patient's face (into the upper lip and sealing under the nose) and oppose the effect of the positive pressure in the plenum chamber 3200 to lift off the face (i.e., Fₚₗₑₙᵤₘ). The force F_{conduit} directed may also be directed at least partially in the superior direction in order to overcome the gravitational force F_{g}.

In some forms, the conduits may provide a force directed into the patient's head when the conduits are filled with pressurized air. For example, the conduits may inflate as pressurized air is conveyed through the conduits. The force may assist in gripping the patient's head. The force may be caused by the inflation of the conduits during normal use. In some forms, the force may provide a cushioning effect to the patient's head. The conduits may be designed in order to limit expansion in order to prevent over-gripping the patient's head.

The position of the patient's head may also change the gripping force of the conduits. For example, if the patient is sleeping on his side, the weight of the patient's head may compress one conduit, and the other conduit (e.g., the lateral portion not between the patient's head and a sleeping surface, like a pillow) may additionally expand in order to keep substantially the same flow rate of pressurized air.

As shown in FIG. 13, some forms of the patient interface 6000 may include a tube or conduit 6320 may be coupled (e.g., removably coupled or permanently coupled) to each conduit connection structure 6500. Each conduit 6320 may convey a flow of pressurized breathable gas (e.g., from the RPT device 4000) toward the patient's airways. The flow of pressurized breathable gas may enter the plenum chamber 6200 through the conduit connection structures 6500 and the plenum chamber inlet ports 6254. The sealing engagement between each conduit connection structure 6500 and plenum chamber inlet port 6254 may limit the flow of pressurized breathable gas from leaking into the ambient through that interface.

In use (see e.g., FIGS. 43, 44, 51, and 52), the conduits 6320 may form conduit headgear 6319, and may extend along the patient's head (e.g., along the patient's cheeks and toward the superior region of the patient's head). The conduits 6320 may take the place of the superior headgear straps of the patient interface 6000. As such, the conduits 6320 may be constructed from a flexible or semi-rigid material (e.g., silicone, textile, etc.), and may be able to flex as the patient dons the patient interface 6000. The length of the conduits 6320 may not be adjustable, and all adjustment may be from the inferior straps.

In some forms, the conduit 6320 may be removably connected to the cushion 6050 using the conduit connection structures 6500. The conduit 6320 may be integrally connected to the conduit connection structures 6500, which in turn connect to the plenum chamber inlet port 6254. The conduit connection structures 6500 may removably connect to the cushion 6050 via the plenum chamber inlet ports 6254 with a mechanical connection (e.g., snap fit, press fit, friction fit, etc.). In other examples, the conduit connection structures 6500 may be permanently connected in the plenum chamber inlet ports 6254, and the conduit 6320 may be removable from the conduit connection structure 6500. In still other examples, the conduit connection structure 6500 may be removably connected from both the conduit 6320 and the plenum chamber inlet ports 6254.

As described above, the groove 6266 may be larger than the plenum chamber inlet port 6254. For example, the groove 6266 may extend past the plenum chamber inlet port 6254 in either lateral direction. The conduit connection structure 6500 may also be larger than the plenum chamber inlet port 6254, and may contact the surface of the groove 6266 when connected to the plenum chamber inlet port 6254. This may allow the conduit connection structure 6500 to be at least partially recessed when connected to the plenum chamber inlet port 6254 in order to maintain a low profile patient interface 6000.

In certain forms, an area formed by the lateral extension of the groove 6266 and the plenum chamber inlet port 6254 may be about the same size as the conduit connection structure 6500. The conduit connection structure 6500 may therefore be secured within the groove as well as within the plenum chamber inlet port 6254. For example, the conduit connection structure 6500 may connect to the groove 6266 using a press fit, friction fit, snap fit, or similar mechanical connection.

Returning to FIG. 13, some forms of the conduits 6320 may include tabs 6324 through which the headgear straps (described below) may be threaded through. The tabs 6324 may be integrally formed with the remainder of the conduits 6320.

With continued reference to FIG. 13, some forms of the conduit 6320 may include a concertina section 6328, which is formed as a series of ridges and grooves on the surface of the conduit 6320. The concertina section 6328 may be biased toward a retracted position (e.g., illustrated in FIG. 13), and may move to an expanded position when the patient dons the conduit headgear 6319. Because the conduit 6320 may be substantially inextensible, the concertina sections 6328 permit the conduit headgear 6319 to stretch in order to fit different sized heads. In other words, the material of the conduit 6320 may be substantially inextensible but the geometric shape of the concertina sections 6328 may permit predetermined extension. Extending the concertina sections 6328 may allow a single sized conduit 6320 to be used with multiple sized heads. For example, the conduit 6320 may be “one-size-fits-all” as a result of the concertina section 6328. Alternatively or additionally, the conduit 6320 and/or the concertina sections 6328 may be manufactured in multiple sizes (e.g., small, medium, large). The patient may select a conduit length that most closely conforms to their head, and the concertina sections 6328 may make small adjustments in order to tailor the fit to the individual patient.

When the patient dons the conduit 6320, the concertina sections 6328 may expand in order to fit around the patient's head. When the patient doffs the conduit 6320, the concertina sections 6328 may return to their initial position (i.e., FIG. 13). The total length that the concertina sections 6328 expand may depend on the size of the patient's head and the initial length of the conduit 6320.

In some forms, the concertina sections 6328 may be superior to the tabs 6324. In other words, the tabs 6324 may be disposed between the conduit connection structure 6500 and the concertina sections 6328.

In the illustrated form, the concertina sections 6328 may not extend entirely to the tabs 6324. In other words, the concertina sections 6328 are spaced apart from the tabs 6324 so that the tabs 6324 are not directly connected to a concertina 6328.

In some forms, the conduit 6320 may include an inlet 6332 for receiving the flow of pressurized air. In the illustrated example, the inlet 6332 may be disposed between the concertina sections 6328. As shown in FIGS. 43 and 51, the inlet 6332 may be positioned at a superior portion of the patient's head in use. For example, the inlet 6332 may overlay the patient's frontal bone and/or parietal bone in use.

In certain forms, the inlet 6332 may be disposed in the middle of the conduit 6320. For example, the conduit 6320 may be symmetric about the inlet 6332 through at least one axis.

In some forms, the conduit 6320 may be a standard component that is interchangeably used with both the full-face cushion 6050 and the nasal cushion 7050. The conduit 6320 may be connected in a similar way to each type of cushion 6050, 7050 so that the conduit 6320 may be easily swapped between cushions 6050, 7050 as desired.

### 5.3.3.2 Rigidizer Arm

As shown in FIG. 14, a rigidizer arm 6340 may be an elongated, rigid member that assists in maintaining the cushion 6050 in an operating position. The rigidizer arm 6340 may contact a side of the patient's head and provide a force to limit slipping of the seal-forming structure 6100 from the patient's nose and/or mouth.

In some forms, the rigidizer arm 6340 is constructed from a rigid material (e.g., plastic). The rigid material may not permit the rigidizer arm 6340 to stretch. Additionally, the rigidizer arm 6340 may be substantially inflexible and may be unable to bend. The rigidizer arm 6340 may be pre-molded into a desired shape in order to fit a patient's head. For example, the rigidizer arms 6340 may be molded with a curved shape to substantially correspond to the shape of the side of the patient's head (e.g., overlaying the masseter muscle and/or the temporal bone).

In certain forms, the rigidizer arm 6340 may be molded in order to conform to a specific patient's head (e.g., the rigidizer arm 6340 is customized).

In some forms, the rigidizer arm 6340 may be flexible along at least one direction. For example, the rigidizer arm 6340 may be flexible about its width and may be inflexible along its length. In other words, the rigidizer arm 6340 may be bendable about an axis along the width of the rigidizer arm 6340, but may be unable to bend about an axis perpendicular to the rigidizer arm 6340. This may allow an individual patient to adjust the rigidizer arm 6340 in order to better fit their individual head.

In certain forms, the rigidizer arm 6340 may remain in the new position after being bent. This may allow a patient adjust the shape of the rigidizer arm 6340 for their specific head and then the arm 6340 will keep the desired shape while in use in order to promote patient comfort.

In some forms, a first end or free end 6342 of the rigidizer arm 6340 may be a free end and a second end 6344 (e.g., opposite to the first end 6342) of the rigidizer arm 6340 may be fixed. The first end 6342 may be curved in order to minimize sharp edges that could cause patient discomfort. The first end 6342 may also overlay the patient's head proximate to the temporal bone, in use. The second end 6344 may be fixed to an arm connection structure 6504. The rigidizer arm 6340 may be connected at an oblique angle relative to the arm connection structure 6504.

In some forms, the arm connection structure 6504 may be similar to the conduit connection structure 6500. For example, the arm connection structure 6504 and the conduit connection structure 6500 may have substantially the same shape. This may allow either the conduit connection structure 6500 or the arm connection structure 6504 to fit into the groove 6266 and connect to the plenum chamber inlet port 6254. The arm connection structure 6504 may connect to the cushion 6050 in substantially the same way as the conduit connection structure 6500 (e.g., via a snap fit, press fit, friction fit, etc.).

In some forms, the arm connection structure 6504 may act as a plug for the plenum chamber inlet port 6254. Unlike the conduit 6320, the rigidizer arm 6340 does not convey pressurized air to the plenum chamber 6200. The rigidized arm 6340 may be used with a “tube down” configuration, where a hose is connected to the vent opening 6402, and conveys air into the plenum chamber 6200 through the vent opening 6402. In this example, air does not need to travel into or out of the plenum chamber inlet ports 6254. Thus, the arm connection structure 6504 may form a seal with the plenum chamber inlet ports 6254 in order to limit airflow into or out of the plenum chamber 6200.

As shown in FIG. 13, a pair of rigidizer arms 6340 are used with the plenum chamber 6200. The rigidizer arms 6340 may be separate from one another so that they may be independently connect in the groove 6266. Additionally, the separate arms 6340 may be separately shaped (e.g., in the molding process or manually by bending) in order to fit different contours on different sides of the patient's head.

In some forms, the rigidizer arms 6340 may be a standard component that is interchangeably used with both the full-face cushion 6050 and the nasal cushion 7050. The rigidizer arms 6340 may be connected in a similar way to each type of cushion 6050, 7050 so that the rigidizer arms 6340 may be easily swapped between cushions 6050, 7050 as desired.

### 5.3.3.3 Headgear Straps

As shown in FIGS. 15 and 16, some forms of the positioning and stabilizing structure 6300 includes headgear 6302, which may be worn by the patient in order to assist in properly orienting the seal-forming structure 6100 against the patient's face (e.g., in order to limit or prevent leaks).

In some forms, the headgear 6302 may be constructed from a textile material, which may be comfortable against the patient's skin. The textile may be flexible in order to conform to a variety of facial contours. Although the textile may include rigidizers along a selected length, which may limit bending, flexing, and/or stretching of the headgear 6302.

In certain forms, the headgear 6302 may be at least partially extensible. For example, the headgear 6302 may include elastic, or a similar extensible material. This may allow the headgear 6302 to stretch while under tension, which may assist in providing a sealing force for the seal-forming structure 6100.

The extensible headgear 6302 may also work similar to the concertina sections 6328. The extensible headgear 6302 may begin in an unstretched position, and may stretch to an expanded position when worn by the patient. In some examples, the headgear 6302 may be “one-size-fits-all” while in other examples, there may be multiple sized headgears 6302 (e.g., small, medium, large) in order to control the total length expansion of the headgear 6302. When the patient removes the headgear 6302, the headgear 6302 may return to its initial position.

In certain forms, only select portions of the headgear 6302 may be extensible. Other portions of the headgear 6302 may be inextensible. For example, portions of the headgear may not be elastic and/or may include rigidizers (e.g., rigidizing thread) in order to limit or prevent the portion of the headgear 6302 from stretching. In still other examples, the entire headgear 6302 may be inextensible. The use of rigidizers may assist in selectively determining where the headgear can stretch, which may create a better fit and/or increase comfort for the patient.

### 5.3.3.3.1 Four-Point Connection

As shown in FIG. 15, some forms of the headgear 6302 may be a four-point connection headgear. This means that the headgear 6302 may connect to four separate places, and therefore may include four different straps providing a tensile force to help maintain the seal-forming structure 6100 in a sealing position.

In some forms, the headgear 6302 may include inferior straps 6304, which may connect to an inferior portion of the cushion 6050. The inferior straps 6304 may extend along the patient's cheek toward a posterior region of the patient's head. For example, the inferior straps 6304 may overlay the masseter muscle on either side of the patient's face. The inferior straps 6304 may therefore contact the patient's head below the patient's ears. The inferior straps 6304 may meet at the posterior of the patient's head, and may overlay the occipital bone and/or the trapezius muscle.

The headgear 6302 may also include superior straps 6305, which may overlay the temporal bones, parietal bone, and/or occipital bone. The superior straps 6305 may also connect to the conduits 6320 (e.g., by interfacing with the tabs 6324).

A rear strap 6307 may extend between the superior straps 6305 and between the inferior straps 6304. The inferior and superior straps 6304, 6305 on a given side (e.g., left or right) may also be connected to the rear strap 6307 adjacent to one another. The height of the rear strap 6307 may therefore be approximately the combined height of the inferior and superior strap 6304, 6305. The rear strap 6307 may overlay the occipital bone and/or the parietal bone in use. This may allow the rear strap 6307 to assist in anchoring the headgear 6302 to the patient's head.

In the illustrated example, the headgear 6302 may be formed with a substantially X-shape. The inferior and superior straps 6304, 6305 may be connected to a rear strap 6307 using stitching, ultrasonic welding, or any similar process.

In some forms, the inferior straps 6304 are connected to a magnetic member 6306. For example, each inferior straps 6304 may be threaded through a magnetic member 6306, so that a length of each inferior strap 6304 may be adjusted. The magnetic members 6306 may removably connect to the magnets 6370 (described below), so that the inferior straps 6304 may be disconnected from the plenum chamber 6200, but the length of the inferior straps 6304 may not be affected.

In some forms, the superior straps 6305 may be connected directly to the tabs 6324 of the conduit 6320. The superior straps 6305 may be threaded through the tabs 6324 in order to adjust the length and control the tensile force of each superior strap 6305.

In some forms, the headgear 6302 may be used only with the full-face cushion 6050 (e.g., because the nasal cushion 7050 does not have four connection points). However, the headgear 6302 may be used interchangeably with the conduit headgear 6319 and the rigidizer arms 6340.

### 5.3.3.3.2 Two-Point Connection

As shown in FIG. 16, some forms of the headgear 7302 may be a two-point connection headgear. This means that the headgear 7302 may connect to two separate places, and therefore may include two different straps providing a tensile force to help maintain the seal-forming structure 7100 in a sealing position.

The headgear 7302 may also include superior straps 7305, which may overlay the temporal bones, parietal bone, and/or occipital bone. The superior straps 7305 may also connect to the conduits 6320 (e.g., by interfacing with the tabs 6324). The superior straps 7305 may contact the patient's head in substantially the same location as the superior straps 6305.

In some forms, the headgear 7302 may not include a separate rear strap. Instead, the superior strap 7305 may serve as a rear strap. For example, the superior strap 7305 may also contact a posterior portion of the patient's head and may overlay the occipital bone and/or the trapezius muscle.

In some forms, the superior strap 7305 may be formed from a continuous piece of material. In other words, the headgear 7302 may not be formed from multiple straps connected together. This may be comfortable for a patient as they will not be in contact with any seams or joints connecting different straps. In other forms, the headgear 7302 may be formed from multiple straps (e.g., two superior straps, a rear strap, etc.) that are connected together.

As shown in FIG. 16, the headgear 7302 may be at least partially bifurcated. For example, a posterior portion 7307 of the headgear 7302 (e.g., configured to contact the posterior portion of the patient's head) may be wider than the surrounding portions of the headgear 7302. An intermediate section 7308 of the posterior portion 7307 may include a slit 7309. A superior section of the posterior portion 7307 may therefore be movable relative to the inferior section as a result of the slit 7309. This may allow the patient to have a larger strap coverage on the posterior region of their head, which may assist in better anchoring the headgear 7302 to the patient's head since there is not inferior strap (e.g., 6304).

In some forms, the headgear 7302 may be used only with the nasal cushion 7050 (e.g., because the full-face cushion 6050 does not have four connection points). However, the headgear 6302 may be used interchangeably with the conduit headgear 6319 and the rigidizer arms 6340.

### 5.3.3.4 Sleeves

A sleeve may be used with the conduit headgear 6319 and/or the rigidizer arms 6340. The sleeve may at least partially surround the conduit headgear 6319 and/or the rigidizer arms 6340. As shown in FIGS. 17 to 24, different shapes of sleeves may be used, which may correspond to different types of positioning and stabilising structures 6300. In some forms, the configuration of the sleeve may be customized to fit a particular user's face. For instance, the sleeves may be configured in a relatively more posterior region of the patient's head.

In some forms, the sleeve may be constructed from a comfortable material. For example, the sleeve may be constructed from a textile material, a foam material, or a combination of the two. The comfortable material may contact the patient in use, and may feel soft against the patient's skin in order to improve patient compliance.

The material may also be flexible in order to assist in donning or doffing the sleeve from the conduit headgear 6319 or the rigidizer arms 6340. For example, the material may allow the sleeve to bend in order to conform to the shape of the conduit headgear 6319 or the rigidizer arms 6340, which may change depending on the shape of an individual patient's head.

In some forms, the sleeve may also be at least partially elastic (e.g., the material may allow the sleeve to stretch). The elastic material may help the sleeve stretch in order to fit around the conduit headgear 6319 or the rigidizer arms 6340. The elastic material may then return to an initial position that is snug against the conduit headgear 6319 or the rigidizer arms 6340 in order to limit the sleeve from sliding while in use.

As described in more detail below, some forms of the sleeves may be specific to a rigidizing element (e.g., a conduit headgear 6319 and/or rigidizer arms 6340). However, the sleeves may assist the rigidizing elements in connecting interchangeably with the version or styles of cushions (e.g., the full-face cushion 6050, the nasal cushion 7050, etc.).

### 5.3.3.4.1 Conduit Sleeve

As shown in FIGS. 17 to 19, one example of a sleeve is a conduit sleeve 6350, which may be usable with the conduit headgear 6319 described above.

As shown in FIG. 17, the conduit sleeve 6350 may include a curved shape that may be similar to the shape of the conduit headgear 6319 shown in FIG. 13. The flexible material used to construct the conduit sleeve 6350 may allow the conduit sleeve 6350 to further curve in order to correspond to the shape of the conduit 6320 (e.g., when worn by the patient).

In some forms, the conduit sleeve 6350 may include a first or superior opening 6352. The superior opening 6352 may be disposed at one end of the conduit sleeve 6350. The superior opening 6352 may be an opening to a passage that extends along at least a portion of the conduit sleeve 6350.

In some forms, the conduit sleeve 6350 may be at least partially elastic proximate to the superior opening 6352. As described above, the elasticity may allow the conduit sleeve 6350 to stretch around the opening 6352 in order to increase the diameter of the opening 6352.

As shown in FIGS. 17 to 19, some forms of the conduit sleeve 6350 may also include an inferior extension 6354. The inferior extension 6354 may be positioned on an opposite end of the conduit sleeve 6350 from the superior opening 6352. The conduit sleeve 6350 may be customized to fit a particular user's face. For instance, the inferior extension 6354 of the conduit sleeve 6350 may be configured in a relatively more posterior region or anterior region of the patient's head.

Some forms of the inferior extension 6354 may include a rigid or semi-rigid piece (e.g., within the sleeve 6350). The rigid or semi-rigid piece may be constructed from a plastic material, or a similar material. Alternatively, the inferior extension 6354 may be stiffened using a manufacturing process (e.g., stitching rigidized thread, flat knitting, using thicker material).

In some forms, the inferior extension 6354 may be separate from the passageway through the conduit sleeve 6350. In other words, a conduit 6320 inserted into the conduit sleeve 6350 may not extend into the inferior extension 6354.

As shown in FIGS. 17 and 18, some forms of the inferior extension 6354 may include a connection member 6356. In the illustrated example, the connection member 6356 may be a magnet, although in other examples, the connection member 6356 may be a different type of connector (e.g., a mechanical fastener, an adhesive, hook and loop material, etc.). The connection member 6356 may also be positioned at an end of the inferior extension 6354, although the connection member 6356 could alternatively be positioned anywhere along the inferior extension 6354.

In some forms, the connection member 6356 (e.g., a magnet) may be removably connected to the magnets 6370 of the headgear 6302. For example, when the conduit sleeves 6350 are connected to the conduit 6320 (described below), the magnets 6370 connected to the inferior straps 6304 may be removably connected to the connection member 6356 in order to provide the tensile force.

As shown in FIGS. 17 and 18, the inferior extension 6354 may be disposed at various locations along the conduit sleeve 6350. For example, as described above, the inferior extension 6354 may be positioned proximate to an end of the conduit sleeve 6350 (see e.g., FIG. 17). Alternatively, the inferior extension 6354 may be positioned in an intermediate location between the superior opening 6352 and the opposite end of the conduit sleeve 6350.

In some forms, changing the position of the inferior extension 6354 may change the position of the connection member 6356. Changing the position of the connection member 6356 may also change the force vector when the inferior straps 6304 are connected (via the magnet 6370) to the conduit sleeve 6350. Changing the position may assist in providing a snug fit for patients with a wide variety of head shapes and sizes.

In some forms, the inferior extension 6354 is formed with the rest of the conduit sleeve 6350. For example, the conduit sleeve 6350 may be formed from a single piece of material. Alternatively, the inferior extension 6354 may be sewn onto the remainder of the conduit sleeve 6350. In either case, the inferior extension 6354 may not be movable from its position. Instead, the conduit sleeve 6350 may be manufactured with the inferior extension 6354 in a variety of positions in order to better accommodate a wider range of head sizes.

Alternatively, the inferior extension 6354 may be removably connected to the remainder of the conduit sleeve 6350 (e.g., using hook and loop material, using a mechanical fastener, using a magnet, etc.). In still other forms, the inferior extension 6354 may be slidable relative to the remainder of the conduit sleeve 6350. In either case, the patient may move the inferior extension 6354 relative to the remainder of the conduit sleeve 6350 into a desired position. The adjustment may be between discrete locations, or between an infinite number of locations depending on the type of adjustment.

As shown in FIG. 19, the conduit sleeve 6350 may also include an inferior opening 6358 that is positioned at an end of the conduit sleeve 6350 opposite to the superior opening 6352. The passageway may extend between the superior and inferior openings 6352, 6358.

In the illustrated example, the inferior opening 6358 may open into a surface of the conduit sleeve 6350. In other words, the inferior opening 6358 may open perpendicularly with respect to the superior opening 6352.

In some forms, the inferior opening 6358 may include elastic material similar to the superior opening 6352. The elastic may enable the inferior opening 6358 to stretch so that the conduit 6320 may fit through the opening.

The example illustrated in FIG. 19 shows the inferior opening 6358 aligned with the inferior extension 6354 (e.g., the inferior extension 6354 is in the position illustrated in FIG. 17). Moving the inferior extension 6354 to another position (e.g., as illustrated in FIG. 18) may not change the position of the inferior opening 6358. In other words, the inferior opening 6358 may always be positioned at an end of the conduit sleeve 6350 even if the inferior extension 6354 is in a new position.

### 5.3.3.4.2 Four-Point Arm Sleeve

As shown in FIGS. 20 to 22, another example of a sleeve is a four-point arm sleeve 6380, which may be usable with the rigidizer arms 6340 described above.

As shown in FIG. 20, the four-point arm sleeve 6380 may include a curved shape that may be similar to the shape of the rigidizer arm 6340 shown in FIG. 14. The flexible material used to construct the four-point arm sleeve 6380 may allow the four-point arm sleeve 6380 to further curve in order to correspond to the shape of the rigidizer arm 6340 (e.g., when worn by the patient and/or went bent by the patient).

As shown in FIGS. 17 to 19, some forms of the four-point arm sleeve 6380 may include an inferior extension 6384. The inferior extension 6384 may be positioned at an end of the four-point arm sleeve 6380.

In the illustrated example, the shape and/or structure of the inferior extension 6384 is substantially the same as the shape of the inferior extension 6354. For example, the inferior extension 6384 may be more rigid as compared to the rest of the four-point arm sleeve 6380 (e.g., as a result of rigidizing thread or rigid material).

As shown in FIGS. 17 and 18, some forms of the inferior extension 6384 may include a connection member 6386. In the illustrated example, the connection member 6386 may be a magnet, although in other examples, the connection member 6386 may be a different type of connector (e.g., a mechanical fastener, an adhesive, hook and loop material, etc.). The connection member 6386 may also be positioned at an end of the inferior extension 6384, although the connection member 6386 could alternatively be positioned anywhere along the inferior extension 6384.

In some forms, the connection member 6386 (e.g., a magnet) may be removably connected to the magnets 6370 of the headgear 6302. For example, when the four-point arm sleeves 6380 are connected to the rigidizer arm 6340 (described below), the magnets 6370 connected to the inferior straps 6304 may be removably connected to the connection member 6386 in order to provide the tensile force.

As shown in FIGS. 20 and 21, the inferior extension 6384 may be disposed at various locations along the four-point arm sleeve 6380. For example, as described above, the inferior extension 6384 may be positioned proximate to an end of the four-point arm sleeve 6380 (see e.g., FIG. 20). Alternatively, the inferior extension 6384 may be positioned toward a center of the four-point arm sleeve 6380 and away from the end (see e.g., FIG. 21).

As described above with respect to the conduit sleeve 6350, changing the position of the inferior extension 6384 may change the position of the connection member 6386. Changing the position of the connection member 6386 may also change the force vector when the inferior straps 6304 are connected (via the magnet 6370) to the four-point arm sleeve 6380. Changing the position may assist in providing a snug fit for patients with a wide variety of head shapes and sizes.

The inferior extension 6384 may be connected to the four-point arm sleeve 6380 in a similar way as the inferior extension 6354 is connected to the conduit sleeve 6350, as described above. For example, the inferior extension 6384 may be permanently connected to the four-point arm sleeve 6380 in a fixed location, or the inferior extension 6384 may be movably or removably connected to the four-point arm sleeve 6380.

As shown in FIG. 22, the four-point arm sleeve 6380 may include an inferior opening 6388 that is positioned at an end of the four-point arm sleeve 6380. The inferior opening 6388 may form an opening to a passageway through the four-point arm sleeve 6380. In the illustrated example, the inferior opening 6388 may open into a surface of the conduit sleeve 6380.

In some forms, the inferior opening 6388 may include elastic material similar to the superior opening 6352 of the conduit sleeve 6350. The elastic may enable the inferior opening 6388 to stretch so that the rigidizer arm 6340 may fit through the opening.

The example illustrated in FIG. 22 shows the inferior opening 6388 aligned with the inferior extension 6384 (e.g., the inferior extension 6384 is in the position illustrated in FIG. 20). Moving the inferior extension 6384 to another position (e.g., as illustrated in FIG. 21) may not change the position of the inferior opening 6388. In other words, the inferior opening 6388 may always be positioned at an end of the four-point arm sleeve 6380 even if the inferior extension 6384 is in a new position.

As shown in FIGS. 20 to 22, the four-point arm sleeve 6380 may be formed as a single sleeve (e.g., unlike the conduit sleeve 6350 that may be part of a pair of sleeves). The four-point arm sleeve 6380 may be shaped similar to the conduit headgear 6319 described above, and may sit on a patient's head in a similar position.

The four-point arm sleeve 6380 may include an inferior opening 6388 on either end. The four-point arm sleeve 6380 may also include an inferior extension 6384 on either end. Thus, the four-point arm sleeve 6380 may be symmetrical similar to the conduit headgear 6319.

In the illustrated example, the inferior openings 6388 may be the only openings of the four-point arm sleeve 6380. In other words, the four-point arm sleeve 6380 may not include a superior opening (e.g., like superior opening 6352). Additionally, the four-point arm sleeve 6380 may include multiple passageways as opposed to a single, connected passageway.

For example, each inferior opening 6388 may be an opening to a single passageway. Each passageway may only include that one opening (i.e., the respective inferior opening 6388), and the passageways may not connect to one another. The length of each passageway may be approximately the length of a rigidizer arm 6340.

As shown in FIG. 22, the four-point arm sleeve 6380 may include a pair of inferior sections 6390 and a superior section 6392. The inferior sections 6390 may be positioned on the right and left sides of the four-point arm sleeve 6380, and may include the respective inferior openings 6388 described above. The inferior sections 6390 may also include a tab 6394, which may be similar to the tab 6324 on the conduit headgear 6319. When the four-point arm sleeve 6380 is worn by the patient, the tabs 6394 may be positioned in substantially the same place on the patient's head as where the tabs 6324 are positioned when the patient wears the conduit headgear 6319. The inferior sections 6390 may also include the passageways.

In some forms, a superior end of each inferior section 6390 (e.g., distal to the inferior opening 6388 and adjacent to the superior section 6392) may include a closed end. For example, an end of the inferior section 6390 opposite to the inferior opening 6388 may be stitched closed in order to form an end of the respective passage.

The superior section 6392 may be connected between the inferior sections 6390. For example, the superior section 6392 may be stitched to the two inferior sections 6390 (although another means of connection may be used). In use, the superior section 6392 may contact a superior region of the patient's head (e.g., overlaying the frontal bone and/or the parietal bone).

The superior section 6392 may be substantially flat and does not have an interior passage. However, some forms of the superior section 6392 may include an outer textile layer with an inner foam layer to provide extra cushioning to the patient's head.

### 5.3.3.4.3 Two-Point Arm Sleeve

As shown in FIGS. 23 and 24, yet another example of a sleeve is a two-point arm sleeve 6380-1, which may be usable with the rigidizer arms 6340 described above.

In some forms, the two-point arm sleeve 6380-1 may be similar to the four-point arm sleeve 6380 described above. Only some similarities and differences may be described below.

As shown in FIG. 23, the two-point arm sleeve 6380-1 may include an inferior opening 6388-1 that is positioned at an end of the two-point arm sleeve 6380-1. The inferior opening 6388-1 may form an opening to a passageway through the two-point arm sleeve 6380-1. In the illustrated example, the inferior opening 6388-1 may open into a surface of the conduit sleeve 6380-1.

In some forms, the inferior opening 6388-1 may include elastic material similar to the superior opening 6352 of the conduit sleeve 6350. The elastic may enable the inferior opening 6388-1 to stretch so that the rigidizer arm 6340 may fit through the opening.

As shown in FIG. 23, the two-point arm sleeve 6380-1 may be formed as a single sleeve (e.g., like the four-point arm sleeve 6380). The two-point arm sleeve 6380-1 may be shaped similar to the conduit headgear 6319 described above, and may sit on a patient's head in a similar position.

As shown in FIG. 24, an alternate version of the two-point arm sleeve 6380-1 may be formed as multiple pieces. For example, the superior section 6392-1 may be formed in two parts, which may be selectively connected to one another (e.g., with hook and loop material, snaps, etc.). This may allow the patient to adjust the size of the sleeve in order to better fit their head. Although it is not illustrated, a similar multi-piece adjustable sleeve may be used with the four-point arm sleeve 6380 described above.

The two-point arm sleeve 6380-1 may include an inferior opening 6388-1 on either end. Thus, the two-point arm sleeve 6380-1 may be symmetrical similar to the conduit headgear 6319.

In the illustrated example, the inferior openings 6388-1 may be the only openings of the two-point arm sleeve 6380-1. In other words, the two-point arm sleeve 6380-1 may not include a superior opening (e.g., like superior opening 6352). Additionally, the two-point arm sleeve 6380-1 may include multiple passageways as opposed to a single, connected passageway.

For example, each inferior opening 6388-1 may be an opening to a single passageway. Each passageway may only include that one opening (i.e., the respective inferior opening 6388-1), and the passageways may not connect to one another. The length of each passageway may be approximately the length of a rigidizer arm 6340.

As shown in FIG. 23, the two-point arm sleeve 6380-1 may include a pair of inferior sections 6390-1 and a superior section 6392-1. The inferior sections 6390-1 may be positioned on the right and left sides of the two-point arm sleeve 6380-1, and may include the respective inferior openings 6388-1 described above. The inferior sections 6390-1 may also include a tab 6394-1, which may be similar to the tab 6324 on the conduit headgear 6319. When the two-point arm sleeve 6380-1 is worn by the patient, the tabs 6394-1 may be positioned in substantially the same place on the patient's head as where the tabs 6324 are positioned when the patient wears the conduit headgear 6319. The inferior sections 6390-1 may also include the passageways.

In some forms, a superior end of each inferior section 6390-1 (e.g., distal to the inferior opening 6388-1 and adjacent to the superior section 6392-1) may include a closed end. For example, an end of the inferior section 6390-1 opposite to the inferior opening 6388-1 may be stitched closed in order to form an end of the respective passage.

The superior section 6392-1 may be connected between the inferior sections 6390-1. For example, the superior section 6392-1 may be stitched to the two inferior sections 6390-1 (although another means of connection may be used). In use, the superior section 6392-1 may contact a superior region of the patient's head (e.g., overlaying the frontal bone and/or the parietal bone).

The superior section 6392-1 may be substantially flat and does not have an interior passage. However, some forms of the superior section 6392-1 may include an outer textile layer with an inner foam layer to provide extra cushioning to the patient's head.

Accordingly, the two-point arm sleeve 6380-1 may be substantially similar to the four-point arm sleeve 6380. However, the two-point arm sleeve 6380-1 may not include an inferior extension or a connection member. Accordingly, straps from the headgear 6302 may only connect to the two-point arm sleeve 6380-1 through the tabs 6394.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

As shown in FIG. 41, a vent 6400 may be used with the patient interface 6000. The vent 6400 may have a substantially similar shape to the vent opening 6402 (e.g., a substantially circular shape).

The vent 6400 may be used with either the full-face patient interface 6000 (e.g., illustrated in FIGS. 7 to 9) or the nasal patient interface 7000 (e.g., illustrated in FIGS. 10 to 12).

With continued reference to FIG. 41, the vent 6400 may include a vent housing or vent body 6404, which may be configured to engage with the vent opening 6402. The vent housing 6404 may be constructed from a rigid material or a semi-rigid material. For example, the vent housing 6404 may be constructed from plastic, metal, or any similar material. The vent housing 6404 may add rigidity to the patient interface 6000 (e.g., to limit unwanted bending that may affect the position of the seal-forming structure 6100 on the patient's face).

The vent housing 6404 may include an anterior surface 6408, a posterior surface 6412, and a groove 6416. The anterior surface 6408 faces away from the patient's face in use, and may be positioned outside the pressurized volume of the plenum chamber 6200. The posterior surface 6412 is disposed opposite to the anterior surface 6408. In use, the posterior surface 6412 may face the patient and may be disposed within the pressurized volume of the plenum chamber 6200. The groove 6416 may be formed between the anterior and posterior surfaces 6408, 6412. A portion of the plenum chamber 6200 may be received within the groove 6416 in order to retain the vent 6400 in position.

In some forms, a diffuser 6448 may be used with the vent housing 6404. The diffuser 6448 may assist with limiting the decibel output from any of the patient interface 6000 (or any other patient interface). Specifically, the diffuser 6448 may assist in limiting the decibel level associated with air output from the patient interface 6000 (e.g., exhaled air), although the diffuser 6448 may limit the decibel level of at any point in the patient interface.

The diffuser 6448 may include a dampening member (not shown) and a cover 6456. The dampening member and the cover 6456 may be coupled to the vent body 6404. The diffuser may be made from a sound dampening material, and the cover 6456 may retain the dampening member in place.

As shown in FIG. 41, some forms of the cover 6456 may not extend to the edge of the vent housing 6404. A gap 6464 may be formed between the edge of the cover 6456 and the surface of the vent body 6404. Airflow may exit the vent 6400 through the gap 6464 in order to exhaust to ambient.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Forehead Support

In one form, the patient interface 3000 includes a forehead support 3700.

### 5.3.8 Anti-Asphyxia Valve

In one form, the patient interface 3000 includes an anti-asphyxia valve.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.3.10 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort.

In some forms, the different styles of cushions, headgear, and sleeves may be used interchangeably with one another in order to form different combinations of patient interfaces. This may be beneficial from a manufacturing prospective because wider variety of patient interfaces may be created using fewer parts. Additionally or alternatively, the various combinations may allow a patient to change styles of patient interface without changing the every component.

For example, air may be delivered to the patient in one of two main ways. First, the patient may receive the flow of pressurized air through conduit headgear 6319. This may be referred to as a “tube up” configuration and may position the connection port at the top of the patient's head. Second, the patient may receive the flow of pressurized air through a hose connected directly to the cushion and positioned in front of the patient's face. This may be referred to as a “tube down” configuration, and may separate the airflow conduits from the positioning and stabilizing device. Even while wearing the same style patient interface (e.g., the full-face patient interface 6000, the nasal patient interface 7000, etc.), some patients may prefer one style of air delivery over the other and/or one style of air delivery may be more conducive for their individual sleep style. Therefore, it may be beneficial to allow a single style of patient interface (e.g., the full-face patient interface 6000, the nasal patient interface 7000) to be used with either the “tube up” or “tube down” configurations.

The patient or clinician may select different components (e.g., cushion, headgear, sleeves, etc.) in order to make a modular assembly for the particular patient. These different components are interchangeable so that the patient may swap out one or more components for a different style (e.g., a nasal mask for a full face mask) of the same component and make a different modular assembly. Alternatively or additionally, a clinician may recommend that a different patient interchange at least some of the components in order to create a different patient interface.

The following description describes the various combinations that may be created by assembling the different components together.

### 5.3.10.1 Assembly

The various elements of the patient interface (e.g., the cushion, the headgear, the sleeve) described above may generally be generic. In other words, they may not be made for a specific configuration (although they may come in different sizes in order to fit different sized patients). This may allow a person assembling the patient interface (e.g., a manufacturer, a clinician, a patient, etc.) to select standard parts in order to assemble a usable patient interface.

### 5.3.10.1.1 Full-Face Tube-Up Configuration

As illustrated in FIGS. 25 to 28-2, the conduit sleeve 6350 may be connected to the conduit headgear 6319. This combination will enable a patient to experience the “tube up” air delivery style with the full face patient interface 6000.

As described above, a pair of conduit sleeves 6350 may be used with the conduit headgear 6319. The following description and related figures are specifically directed to the connection between one conduit sleeve 6350 and one conduit 6320 of the conduit headgear 6319. The other conduit sleeve 6350 would connect to the other conduit 6320 of the conduit headgear 6319 in substantially the same way.

As illustrated in FIG. 25, the conduit 6320 and the conduit sleeve 6350 may begin separated from one another. The patient may position the superior opening 6352 of the conduit sleeve 6350 proximate to the conduit connection structure 6500 of the conduit 6320.

In some forms, the conduit sleeve 6350 may be substantially flat when not in use. As described above, at least some portions of the conduit sleeve 6350 may include elastic material (e.g., around the superior opening 6352). A patient may stretch the area around the superior opening 6352 in order to create a wide enough space to accommodate the conduit connection structure 6500.

The configuration in FIG. 25 shows an inner surface of the conduit 6320 and a posterior surface of the conduit sleeve 6350. In other words, the surfaces of the conduit 6320 and the conduit sleeve 6350 illustrated in FIG. 25 would both face and/or contact the patient in use. During assembly, the connecting the conduit 6320 and the conduit sleeve 6350 are oriented in the same direction so that the conduit connection structure 6500 can eventually connect to the plenum chamber 6200.

As shown in FIG. 26, the superior opening 6352 of the conduit sleeve 6350 may receive the conduit clip structure 6500, and may be positioned at least partially along the length of the conduit 6320. The elastic portion of the conduit sleeve 6350 that previously allowed the superior opening 6352 to stretch and receive the conduit clip structure 6500 may return toward its original position. This may create a snug engagement between the conduit sleeve 6350 and the conduit 6320 (which may limit relative movement between the conduit sleeve 6350 and the conduit 6320 in order to limit slipping while worn by the patient). The patient may need to continue to stretch the superior opening 6352 in order to allow the conduit sleeve 6350 to continue to move along the conduit 6320.

With continued reference to FIG. 26, the conduit clip structure 6500 may be fully contained within the conduit sleeve 6350. In other words, the conduit clip structure 6500 may not be aligned with the inferior opening 6358 (and is not exposed to a patient). Therefore, a patient may be unable to connect the conduit clip structure 6500 to the plenum chamber 6200 in this position.

As shown in FIG. 27, the conduit sleeve 6350 may continue to be slid along the surface of the conduit 6320. Specifically, the superior opening 6352 may be positioned further from the conduit clip structure 6500 (e.g., closer toward the concertina section 6328). As described above, the patient may continue to stretch the superior opening 6352 in order to facilitate movement along the conduit 6320.

FIG. 27 also illustrates the conduit clip structure 6500 exposed to a patient. Specifically, the conduit clip structure 6500 may be aligned with the inferior opening 6358. This may occur when the conduit 6320 is slid fully through the conduit sleeve 6350. Like the superior opening 6352, the inferior opening 6358 may be biased to a normally closed position, but may be formed with an elastic material. The patient may stretch the material around the inferior opening 6358 in order to allow the conduit clip structure 6500 to be positioned through the inferior opening 6358.

When the conduit clip structure 6500 is positioned in the orientation of FIG. 27, the conduit 6320 may not be positioned in the inferior extension 6354. As described above, the inferior extension 6354 may be sectioned off (e.g., by stitching, ultrasonic welding, etc.) from the passageway of the conduit sleeve 6350.

As shown in FIG. 28, the conduit sleeve 6350 may be fully connected to the conduit 6320. Specifically, the conduit clip structure 6500 may be positioned through the inferior opening 6358 (e.g., after the inferior opening 6358 was stretched in FIG. 27). In some forms, the elastic of the inferior opening 6358 may relax and return the shape of the inferior opening toward its initial position (e.g., so that it is snug against the conduit clip structure 6500 and sliding is limited). In this position, the conduit clip structure 6500 may be able to connect to a plenum chamber 6200 without substantial interference from the conduit sleeve 6350.

As shown in FIGS. 28-1 and 28-2, after the conduit sleeve 6350 is fully connected, the conduit clip structure 6500 may remain exposed so that it can connect to the cushion 6050, 7050. The conduit clip structure 6500 may extend from the conduit sleeve 6350 in order to limit interferences when connecting the conduit 6320 to the cushion 6050, 7050.

### 5.3.10.1.2 Full-Face Tube-Down Configuration

As illustrated in FIGS. 29 to 33-2, the four-point arm sleeve 6380 may be connected to the rigidizer arm 6340. This combination will enable a patient to experience the “tube down” air delivery style with the full face patient interface 6000.

As described above, a single four-point arm sleeve 6380 may be used with the pair of rigidizer arms 6340. The following description and related figures are specifically directed to the connection between one rigidizer arm 6340 and the four-point arm sleeve 6380. The other rigidizer arm 6340 would connect to the four-point arm sleeve 6380 in substantially the same way.

As illustrated in FIG. 29, the rigidizer arm 6340 and the four-point arm sleeve 6380 may begin separated from one another. The patient may position the inferior opening 6388 of the four-point arm sleeve 6380 proximate to the first end 6342 of the rigidizer arm 6340.

In some forms, the four-point arm sleeve 6380 may be substantially flat when not in use. As described above, at least some portions of the four-point arm sleeve 6380 may include elastic material (e.g., around the inferior opening 6388). A patient may stretch the area around the inferior opening 6388 in order to create a wide enough space to accommodate the first end 6342 of the rigidizer arm 6340.

In some forms, the rigidizer arm 6340 (e.g., particularly the first end 6342) may be substantially flat. This may allow a patient to insert the rigidizer arm 6340 into the four-point arm sleeve 6380 without substantially stretching the inferior opening 6388 (e.g., the first end 6342 may be smaller than the inferior opening 6388 and may be able to slide in without stretching the opening 6388).

The configuration in FIG. 29 shows an inner surface of the four-point arm sleeve 6380 and a posterior surface of the rigidizer arm 6340. In other words, the surfaces of the rigidizer arm 6340 and the four-point arm sleeve 6380 illustrated in FIG. 29 would both face and/or contact the patient in use. During assembly, the connecting the four-point arm sleeve 6380 and the rigidizer arm 6340 are oriented in the same direction so that the arm connection structure 6504 can eventually connect to the plenum chamber 6200.

As shown in FIGS. 30 and 31, the inferior opening 6388 of the four-point arm sleeve 6380 may receive the first end 6342 of the rigidizer arm 6340, and may be positioned at least partially along the length of the rigidizer arm 6340. The elastic portion of the four-point arm sleeve 6380 that may allow the inferior opening 6388 to stretch and receive the free end 6342 may return toward its original position (if it was initially stretched).

In some forms, the rigidizer arm 6340 may have a width substantially no greater than the four-point arm sleeve 6380. The patient may not need to continuously stretch the four-point arm sleeve 6380 in order to move the rigidizer arm 6340 through the four-point arm sleeve 6380. Instead, the rigidizer arm 6340 may slide through the four-point arm sleeve 6380 without substantial resistance. The flexible material of the four-point arm sleeve 6380 may be able to bend along the shape of the rigidizer arm 6340 (if the rigidizer arm 6340 is bent to better conform to the patient's face). For example, between FIGS. 30 and 31, the patient may be able to slide the rigidizer arm 6340 through the four-point arm sleeve 6380 without substantially needed to stretch the inferior opening 6388.

With continued reference to FIGS. 30 and 31, the arm clip structure 6504 may be fully outside of the four-point arm sleeve 6380. In other words, the arm clip structure 6504 may not have passed through the inferior opening 6388 (and is exposed to a patient).

As shown in FIG. 32, the four-point arm sleeve 6380 may continue to be slid along the surface of the rigidizer arm 6340. The inferior opening 6388 may be positioned closer to the arm clip structure 6504. Specifically, the arm clip structure 6504 may be positioned at least partially through the inferior opening 6388 while remaining exposed to a patient. The arm clip structure 6504 may be aligned with the inferior opening 6388. This may occur when the rigidizer arm 6340 is slid fully through the four-point arm sleeve 6380. As previously described, the inferior opening 6388 may be biased to a normally closed position, but may be formed with an elastic material. The patient may stretch the material around the inferior opening 6388 in order to allow the arm clip structure 6504 to be positioned through the inferior opening 6388.

When the arm clip structure 6504 is positioned in the orientation of FIG. 32, the rigidizer arm 6340 (e.g., the arm clip structure 6504) may not be positioned in the inferior extension 6384. As described above, the inferior extension 6384 may be sectioned off (e.g., by stitching, ultrasonic welding, etc.) from the passageway of the four-point arm sleeve 6380.

As shown in FIG. 33, the four-point arm sleeve 6380 may be fully connected to the rigidizer arm 6340. Specifically, the arm clip structure 6504 may be positioned partially through and surrounded by the inferior opening 6388 (e.g., after the inferior opening 6388 was stretched in FIG. 32). In some forms, the elastic of the inferior opening 6388 may relax and return the shape of the inferior opening toward its initial position (e.g., so that it is snug against the arm clip structure 6504 and sliding is limited). In this position, the arm clip structure 6504 may be able to connect to a plenum chamber 6200 without substantial interference from the four-point arm sleeve 6380.

With continued reference to FIG. 33, the first end 6342 (see e.g., FIG. 29) of the rigidizer arm 6340 may be positioned at an end of the inferior section 6390 adjacent to the superior section 6392. As described above, the superior section 6392 may not include a passage for receiving the rigidizer arm 6340. The length of the inferior section 6390 may be substantially similar or equal to the length of the rigidizer arm 6340 so that the rigidizer arm 6340 abuts or is substantially close to abutting the end of the inferior section 6390.

As shown in FIGS. 33-1 and 33-2, after the four-point arm sleeve 6380 is fully connected, the arm clip structure 6504 may remain exposed so that it can connect to the cushion 6050, 7050. The arm clip structure 6504 may extend from the four-point arm sleeve 6380 in order to limit interferences when connecting the rigidizer arm 6340 to the cushion 6050, 7050 (see e.g., FIGS. 39 and 40).

### 5.3.10.1.3 Nasal Tube-Down Configuration

As illustrated in FIGS. 34 to 38-2, the two-point arm sleeve 6380-1 may be connected to the rigidizer arm 6340. This combination will enable a patient to experience the “tube down” air delivery style with the nasal patient interface 7000.

As described above, a single two-point arm sleeve 6380-1 may be used with the pair of rigidizer arms 6340. The following description and related figures are specifically directed to the connection between one rigidizer arm 6340 and the two-point arm sleeve 6380-1. The other rigidizer arm 6340 would connect to the two-point arm sleeve 6380-1 in substantially the same way.

As illustrated in FIG. 34, the rigidizer arm 6340 and the two-point arm sleeve 6380-1 may begin separated from one another. The patient may position the inferior opening 6388-1 of the two-point arm sleeve 6380-1 proximate to the first end 6342 of the rigidizer arm 6340.

In some forms, the two-point arm sleeve 6380-1 may be substantially flat when not in use. As described above, at least some portions of the two-point arm sleeve 6380-1 may include elastic material (e.g., around the inferior opening 6388-1). A patient may stretch the area around the inferior opening 6388-1 in order to create a wide enough space to accommodate the first end 6342 of the rigidizer arm 6340.

In some forms, the rigidizer arm 6340 (e.g., particularly the first end 6342) may be substantially flat. This may allow a patient to insert the rigidizer arm 6340 into the two-point arm sleeve 6380-1 without substantially stretching the inferior opening 6388-1 (e.g., the first end 6342 may be smaller than the inferior opening 6388-1 and may be able to slide in without stretching the opening 6388-1).

The configuration in FIG. 34 shows an inner surface of the two-point arm sleeve 6380-1 and a posterior surface of the rigidizer arm 6340. In other words, the surfaces of the rigidizer arm 6340 and the two-point arm sleeve 6380-1 illustrated in FIG. 34 would both face and/or contact the patient in use. During assembly, the connecting the two-point arm sleeve 6380-1 and the rigidizer arm 6340 are oriented in the same direction so that the arm connection structure 6504 can eventually connect to the plenum chamber 6200.

As shown in FIGS. 35 and 36, the inferior opening 6388-1 of the two-point arm sleeve 6380-1 may receive the first end 6342 of the rigidizer arm 6340, and may be positioned at least partially along the length of the rigidizer arm 6340. The elastic portion of the two-point arm sleeve 6380-1 that may allow the inferior opening 6388-1 to stretch and receive the free end 6342 may return toward its original position (if it was initially stretched).

In some forms, the rigidizer arm 6340 may have a width substantially no greater than the two-point arm sleeve 6380-1. The patient may not need to continuously stretch the two-point arm sleeve 6380-1 in order to move the rigidizer arm 6340 through the two-point arm sleeve 6380-1. Instead, the rigidizer arm 6340 may slide through the two-point arm sleeve 6380-1 without substantial resistance. The flexible material of the two-point arm sleeve 6380-1 may be able to bend along the shape of the rigidizer arm 6340 (if the rigidizer arm 6340 is bent to better conform to the patient's face). For example, between FIGS. 35 and 36, the patient may be able to slide the rigidizer arm 6340 through the two-point arm sleeve 6380-1 without substantially needing to stretch the inferior opening 6388-1.

With reference to FIG. 36, the arm clip structure 6504 may be positioned fully outside the two-point arm sleeve 6380-1. In other words, the arm clip structure 6504 may not have passed through the inferior opening 6388-1 (and is exposed to a patient).

As shown in FIG. 37, the two-point arm sleeve 6380-1 may continue to be slid along the surface of the rigidizer arm 6340. The inferior opening 6388-1 may be positioned closer to the arm clip structure 6504. Specifically, the arm clip structure 6504 may be positioned at least partially through the inferior opening 6388-1 while remaining exposed to a patient. The arm clip structure 6504 may be aligned with the inferior opening 6388-1. This may occur when the rigidizer arm 6340 is slid fully through the two-point arm sleeve 6380-1. As previously described, the inferior opening 6388-1 may be biased to a normally closed position, but may be formed with an elastic material. The patient may stretch the material around the inferior opening 6388-1 in order to allow the arm clip structure 6504 to be positioned through the inferior opening 6388-1.

As shown in FIG. 38, the two-point arm sleeve 6380-1 may be fully connected to the rigidizer arm 6340. Specifically, the arm clip structure 6504 may be positioned partially through and surrounded by the inferior opening 6388-1 (e.g., after the inferior opening 6388-1 was stretched in FIG. 37). In some forms, the elastic of the inferior opening 6388-1 may relax and return the shape of the inferior opening toward its initial position (e.g., so that it is snug against the arm clip structure 6504 and sliding is limited). In this position, the arm clip structure 6504 may be able to connect to a plenum chamber 6200 without substantial interference from the two-point arm sleeve 6380-1.

The first end 6342 of the rigidizer arm 6340 may be positioned at an end of the inferior section 6390-1 adjacent to the superior section 6392-1 (see e.g., FIG. 23). As described above, the superior section 6392-1 may not include a passage for receiving the rigidizer arm 6340. The length of the inferior section 6390-1 may be substantially similar or equal to the length of the rigidizer arm 6340 so that the rigidizer arm 6340 abuts or is substantially close to abutting the end of the inferior section 6390-1.

As shown in FIGS. 38-1 and 38-2, after the two-point arm sleeve 6380-1 is fully connected, the arm clip structure 6504 may remain exposed so that it can connect to the cushion 6050, 7050. The arm clip structure 6504 may extend from the two-point arm sleeve 6380-1 in order to limit interferences when connecting the rigidizer arm 6340 to the cushion 6050, 7050.

### 5.3.10.1.4 Nasal Tube-Up Configuration

A patient seeking to use the “tube up” configuration as part of the nasal patient interface 7000 does not need to use a sleeve in order to complete the assembly. The patient may connect the conduit headgear 6319 (e.g., via the arm clip structure 6504) directly to the plenum chamber 7200.

In some forms, the patient may connect a sleeve (not shown) to the conduits 6320 of the conduit headgear 6319. The sleeve may be similar to the conduit sleeve 6350 and may be formed from a textile material. Some forms of the sleeve may also be at least partially elastic. Additionally, each conduit 6320 may include a separate sleeve. The sleeve may be used for patient comfort (e.g., because the textile material may be comfortable against the patient's skin).

### 5.3.10.1.5 Connection

FIGS. 39 and 40 illustrate a process of connecting a rigidizer arm 6340 with a four-point arm sleeve 6380 to a full-face plenum chamber 6200. The following description will specifically reference that configuration. However, the same description will be applicable to the other configurations (e.g., because the conduit clip structure 6500 and the arm clip structure 6504 are the same shape and connect to the plenum chambers 6200, 7200 in the same manner).

As shown in FIG. 39, the air circuit 4170 is connected to the vent opening 6402 indicating that the cushion 6050 is being assembled in the tube down configuration. Thus, the patient may select the rigidizer arm 6340 (e.g., as a result of the tube down configuration) and the four-point arm sleeve 6380 (e.g., as a result of the full-face cushion 6050).

The four-point arm sleeve 6380 and the rigidizer arm 6340 may be connected as described above. Once connected, the assembly may be connected to the plenum chamber inlet port 6254.

With continued reference to FIG. 39, the inferior opening 6388-1 (see e.g., FIGS. 34 to 37) of the four-point arm sleeve 6380 may be positioned around the arm clip structure 6504 in order to avoid obstructing the arm clip structure 6504.

In the illustrated example, the arm clip structure 6504 may include at least one projection 6508 extending from the surface of the rigidizer arm 6340. The at least one projection 6508 may have a similar shape as the plenum chamber inlet port 6254 and may be shaped to fit within the plenum chamber inlet port 6254.

In some forms, the at least one projection 6508 may fit into the plenum chamber inlet port 6254 with a snap-fit, a press fit, and/or a friction fit. The connection may create a substantially air-tight engagement, while also allowing the connection to be removable.

In some forms, the arm clip structure 6504 also includes at least one protrusion 6512 (one illustrated in FIGS. 39 and 40), which may be connected to the at least one projection 6508. For example, the protrusion 6512 may be positioned at an end of the at least one projection 6508 and may extend in a direction substantially perpendicular to the at least one projection 6508 (although other positions and angles may be used). The protrusion 6512 may fit into a groove 6286 adjacent to the plenum chamber inlet port 6254.

In certain forms, the engagement between the protrusion 6512 and the groove 6286 may assist in properly orienting the at least one projection 6508 in the plenum chamber inlet port 6254. For example, the arm clip structure 6504 may only fit in one direction so that the headgear can properly connect and so that the interface is substantially air-tight.

In certain forms, the engagement between the protrusion 6512 and the groove 6286 may assist in further connecting the arm clip structure 6504 to the plenum chamber 6200. For example, the protrusion 6512 may fit into the groove 6286 with a press fit, snap fit, and/or frictional fit.

As illustrated in FIGS. 39 and 40, the patient may align the arm clip structure 6504 with the plenum chamber inlet port 6254 so that the protrusion 6512 is aligned with the groove 6286. The patient may then move the arm clip structure 6504 so that the at least one projection 6508 fits within the plenum chamber inlet port 6254, and the arm clip structure 6504 is connected to the plenum chamber 6200.

### 5.3.10.2 Assembled Patient Interfaces

As illustrated in FIGS. 43 to 58, the various elements described above may be combined into four different patient interfaces. The different patient interfaces may allow patients to use different styles based on their individual comfort. The modularity of the different elements (e.g., the ability to be used in multiple styles of patient interfaces) may simplify manufacturing and/or may allow a patient to more easily switch between styles of patient interfaces.

### 5.3.10.2.1 Full-Face Tube-Up Interface

As illustrated in FIGS. 43 to 46, the patient may wear the cushion 6050 in a tube-up configuration with the conduit headgear 6319, the four-point headgear 6302, and the conduit sleeves 6350.

The elements may be assembled as described above. For example, the conduit sleeves 6350 may be connected to the conduits 6320 of the conduit headgear 6319. The conduits 6320 (via the conduit connection structure 6500), may be used to connect the conduit headgear 6319 to the cushion 6050. The conduit sleeves 6350 provide the magnets 6356 in order to connect to the magnets 6370 (see e.g., FIG. 15) of the four-point headgear 6302.

As illustrated in FIGS. 43 and 44, the four-point headgear 6302 may connect in four separate locations in order to provide a tensile force that maintains the cushion 6050 in a sealing position on the patient's head.

For example, the inferior straps 6304 (e.g., via the magnetic members 6306) may removably connect to the magnets 6370 of the conduit sleeves 6350. In use, each inferior strap 6304 may contact the patient's cheek (e.g., overlaying the masseter muscle). The inferior straps 6304 may also extend below the patient's ears.

The tensile force may be provided along the inferior straps 6304 toward a posterior region of the patient's head (e.g., toward the occipital bone). The tensile force may pull the cushion 6050 into the patient's head. Specifically, the tensile force may be applied at the magnets 6370 on the conduit sleeve 6350. Because the inferior extensions 6354 are constructed from a rigid or semi-rigid material, the inferior extensions 6354 may remain substantially fixed when the tensile force is applied (e.g., as a result of wearing the four-point headgear 6302 connected to the cushion 6050).

In some forms, the magnets 6370 may be positioned proximate to an inferior portion of the cushion 6050 when the conduit headgear 6319 is connected. This may cause the tensile force to specifically act on the inferior region of the cushion 6050. In other words, the tensile force provided by the inferior straps 6304 in order to maintain the first seal forming structure 6101 in a sealing position. Of course, the tensile force from the inferior straps 6304 may also assist in maintaining the second seal forming structure 6102 in a sealing position.

With continued reference to FIGS. 43 and 44, the conduits 6320 may be positioned along the patient's cheek and may extend to a location superior to the patient's ears. For example, each conduit 6320 may extend along a side of the patient's head toward the crown of the patient's head. This may cause each conduit 6320 to overlay the sphenoid bone and/or the temporal bone, and to extend toward the frontal bone and/or the parietal bone.

The conduit sleeves 6350 may cover a portion of the conduit headgear 6319. In other words, the conduit sleeve 6350 and not the conduit headgear 6319 may contact the patient along at least a portion of the length of the conduit headgear 6319. As shown in FIGS. 43 and 44, a superior portion of each conduit 6320 may contact the patient's head, while an interior portion may be covered by the conduit sleeve 6350 (so that the conduit sleeve 6350 contacts the patient along the patient's cheeks).

The second seal forming structure 6102 may contact the underside of the patient's nose. For example, the second seal forming structure 6102 may avoid contact with the patient's nasal ridge and may contact the patient's nose at or below the pronasale and against the columella. The conduits 6320 may provide an additional tensile force that is directed in both the superior and posterior directions (e.g., as viewed in FIG. 44). In this way, the second seal forming structure 6102 may be pulled up onto an underside of the patient's nose in a sealing position (e.g., as a result of the superior directed force). The conduits 6320 may also pull the cushion 6050 into the patient's head in order to retain the cushion 6050 in the sealing position (and assist with sealing the first seal forming structure 6101).

In some forms, the conduits 6320 may be generally inextensible and may be structured in order to provide a tensile force when worn by the patient. As described above, the length of the conduits 6320 may be smaller than the patient's head so that they provide tension when worn. Even when the concertina sections 6328 expand, the conduits 6320 may fit snuggly against the patient's head and the passage through the conduits 6320 may remain wide enough to allow a continuous flow of air.

The conduit headgear 6319 may include a tab 6324 on either conduit 6320. As illustrated in FIG. 44, a superior strap 6305 may be connected to the respective tab 6324. When the conduit headgear 6319 is worn by the patient, the tabs 6324 may be superior to the patient's ears. This may allow the superior straps 6305 connected to the tabs 6324 to also be positioned superior to the patient's ears. For example, each tab 6324 may be positioned proximate to a temporal bone, and each superior strap 6305 may overlay the temporal bone and extend toward the occipital bone.

The superior straps 6305 may provide a tensile force directed toward the posterior portion of the patient's head. In the illustrated example, the superior straps 6305 may extend in an inclined direction toward an inferior region of the patient's head (e.g., toward the occipital bone). The tensile force may maintain the conduits 6320 in a desired position (e.g., so that they do not slip over the patient's eyes) and/or provide an additional force to maintain the seal-forming structure 6100 in the sealing position.

In some forms, the positioning and stabilizing structure 6300 provides a positioning and stabilizing force F_{PSS} that assists in maintaining the cushion 6050 in the sealing position on the patient's face. The positioning and stabilizing force F_{PSS} may be the resultant force from the various force vectors of the different elements of the positioning and stabilizing structure 6300.

In the illustrated example in FIG. 44, the patient is oriented in an upright position so force F_{PSS} acts to oppose the external forces acting in that orientation. In other orientations of the patient, the magnitude of the force F_{PSS} (as well as any other force) may change.

For example, the conduit headgear 6319 may provide a tube force F_{tube} in order to hold the seal-forming structure 6100 against the patient's face. As described above, the conduit headgear 6319 may be sized so that it fits snuggly against the patient's head. The concertina sections 6328 may extend in order to provide the necessary F_{tube}. The tube force F_{tube} may be directed in the superior and/or posterior directions.

The force F_{tube} may also be directed at least partially in the superior direction in order to overcome the gravitational force F_{g}. The gravitational force F_{g} may be specifically shown for the seal-forming structure 6100 and the plenum chamber 6200, but gravity would act on the entirely of the patient interface 6000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the cushion 6050 in the inferior direction (as viewed in FIG. 44), the frictional force F_{f} would act in the superior direction (e.g., against a patient's face). For example, the patient may experience the frictional force F_{f} against his lip superior (and/or other surfaces of the patient's face in contact with the seal-forming structure 6100) in order to oppose the motion in the inferior direction (which may help to stabilizing the cushion 6050 in place). Although the frictional force F_{f} is shown specifically opposing the gravitational force F_{g} of the cushion 6050, components of an overall frictional force (not shown) would also oppose the gravitational force F_{g} associated with the positioning and stabilizing structure 6300 and any other portions of the patient interface 6000. A force of friction can act along any place where the patient interface 6000 contacts the patient's skin (or hair). The frictional force F_{f} extends in the opposite direction of the gravitational force F_{g} and along the patient's skin (or hair).

Additionally, headgear straps may individually provide a strap force Fₛₜᵣₐₚ in order to hold the seal-forming structure 6100 against the patient's face. Each strap may provide a different strap force Fₛₜᵣₐₚ based on how tight the individual strap was tightened.

In some forms, the sum of the various forces may equal zero so that the patient interface 6000 is at equilibrium (e.g., not moving along the patient's face while in use). Specifically, the gravitational force F_{g} and the blowout force Fₚₗₑₙᵤₘ tend to move the seal-forming structure 6100 away from the desired sealing position. The positioning and stabilizing force F_{PSS} is applied in order to counteract the gravitational force F_{g} and the blowout force Fₚₗₑₙᵤₘ (as well as any frictional forces F_{f}) and keep the seal-forming structure 6100 properly situated. Although the positioning and stabilizing force F_{PSS} may exceed the sum of the other forces and still maintain the seal-forming structure 6100 in an appropriate sealing position, patient comfort may be sacrificed. Maximum patient comfort may be achieved when the net force on the patient interface 6000 is zero and the positioning and stabilizing force F_{PSS} is approximately strong enough to achieve this. As described below, various positions of the patient's head while using the patient interface 6000 may determine the positioning and stabilizing force F_{PSS} necessary to achieve equilibrium.

The magnitude of the forces may change as a result of different in use positions (e.g., when the patient is sleeping in different positions). For example, FIG. 44-1 illustrates the forces acting while the patient sleeps on their back. In this position, the gravitational force F_{g} is directed toward the patient's face and the frictional force F_{f} is directed away from the patient's face. The gravitational force F_{g} may therefore be directed in a similar direction as the force F_{PSS}. Therefore, the gravitational force F_{g} may not oppose the positioning and stabilising force F_{PSS}, which may allow the positioning and stabilising structure 6300 to be less tight and maintain the same sealing force (e.g., an improve patient comfort).

Similarly, FIG. 44-2 illustrates the forces acting while the patient sleeps on their side. In the illustrated example, the gravitational force F_{g} is represented as an “x” within a circle in order to show the force directed into the page. The gravitational force F_{g} may be directed substantially perpendicular to the plenum chamber force Fₚₗₑₙᵤₘ and/or the positioning and stabilizing force F_{PSS}. As described above, the positioning and stabilizing force F_{PSS} may have to counteract both the gravitational force F_{g} and the plenum chamber force Fₚₗₑₙᵤₘ. Also, the plenum chamber 6200 and/or the conduit headgear 6319 may tend to compress on the inferior side, yet be in tension on the superior side. The frictional force F_{f} may still be directed opposite of the gravitational force F_{g} (e.g., out of the page).

In some forms, a tube drag force may provide an additional force on the system. In the tube-up configuration illustrated in FIGS. 44 to 44-2, a tube drag force may act at a superior region of the patient's head. Depending on the orientation of the tube (e.g., the angular position of the swivel), the tube drag force may assist the positioning and stabilising force F_{PSS} and/or oppose the positioning and stabilising force F_{PSS}. This may change throughout use as the position of the tube changes.

### 5.3.10.2.2 Full-Face Tube-Up Interface

As illustrated in FIGS. 47 to 50, the patient may wear the cushion 6050 in a tube-down configuration with the rigidizer arms 6340, the four-point headgear 6302, and the four-point arm sleeve 6380. The same cushion 6050 and the four-point headgear 6302 may be used in both full-face configurations (e.g., FIGS. 43-46 and 47-50). In other words, the rigidizer arms 6340 may be interchanged with the conduits 6320 in order to form the tube-down version (i.e., as opposed to the tube up version described above). The four-point arm sleeve 6380 may be used in place of the conduit sleeves 6350 to facilitate this interchange.

The elements may be assembled as described above. For example, the four-point arm sleeve 6380 may be connected to the rigidizer arms 6340. The rigidizer arms 6340 (via the arm connection structure 6504), may be used to connect the rigidizer arms 6340 to the cushion 6050. The four-point arm sleeve 6380 provide the magnets 6386 in order to connect to the four-point headgear 6302.

As illustrated in FIGS. 47 and 48, the four-point headgear 6302 may connect in four separate locations in order to provide a tensile force that maintains the cushion 6050 in a sealing position on the patient's head.

For example, the inferior straps 6304 (e.g., via the magnetic members 6306) may removably connect to the magnets 6386 of the four-point arm sleeve 6380. In use, each inferior strap 6304 may contact the patient's cheek (e.g., overlaying the masseter muscle). The inferior straps 6304 may also extend below the patient's ears.

The tensile force may be provided along the inferior straps 6304 toward a posterior region of the patient's head (e.g., toward the occipital bone). The tensile force may pull the cushion 6050 into the patient's head. Specifically, the tensile force may be applied at the magnets 6386 on the four-point arm sleeve 6380. Because the inferior extensions 6384 are constructed from a rigid or semi-rigid material, the inferior extensions 6384 may remain substantially fixed when the tensile force is applied (e.g., as a result of wearing the four-point headgear 6302 connected to the cushion 6050).

In some forms, the magnets 6386 may be positioned proximate to an inferior portion of the cushion 6050 when the rigidizer arms 6340 are connected. This may cause the tensile force to specifically act on the inferior region of the cushion 6050. In other words, the tensile force provided by the inferior straps 6304 in order to maintain the first seal forming structure 6101 in a sealing position. Of course, the tensile force from the inferior straps 6304 may also assist in maintaining the second seal forming structure 6102 in a sealing position.

With continued reference to FIGS. 47 and 48, the rigidizer arms 6340 (e.g., within the inferior sections 6390) may be positioned along the patient's cheek and may extend to a location superior to the patient's ears. For example, each rigidizer arm 6340 may extend along a side of the patient's head toward the crown of the patient's head. This may cause each rigidizer arm 6340 to overlay the sphenoid bone and/or the temporal bone, and to extend toward the frontal bone and/or the parietal bone. In some forms, the rigidizer arm 6340 may extend along a similar path of the patient's face as the conduit 6320.

In these figures, the rigidizer arms 6340 may be covered by the four-point arm sleeve 6380 so that each rigidizer arm 6340 is positioned within the interior of the four-point arm sleeve 6380. The rigidizer arms 6340 are therefore not visible and do not directly contact the patient.

The second seal forming structure 6102 may contact the underside of the patient's nose. For example, the second seal forming structure 6102 may avoid contact with the patient's nasal ridge and may contact the patient's nose at or below the pronasale and against the columella. The four-point arm sleeve 6380 and the rigidizer arms 6340 (e.g., provided within the four-point arm sleeve 6380 as illustrated in the steps of FIGS. 29-33) may provide an additional tensile force that is directed in both the superior and posterior directions (e.g., as viewed in FIG. 48). In this way, the second seal forming structure 6102 may be pulled up onto an underside of the patient's nose in a sealing position (e.g., as a result of the superior directed force). The four-point arm sleeve 6380 and/or the rigidizer arms 6340 may also pull the cushion 6050 into the patient's head in order to retain the cushion 6050 in the sealing position (and assist with sealing the first seal forming structure 6101).

In some forms, the rigidizer arms 6340 may be generally inextensible and may be structured in order to provide a tensile force when worn by the patient. The four-point arm sleeve 6380 may also be at least partially inextensible along its length (although it may be able to stretch). The superior section 6392 of the four-point arm sleeve 6380 may be adjustable (e.g., via hook and loop material) in order tighten the four-point arm sleeve 6380 against the patient's head and create the tensile force.

The four-point arm sleeve 6380 may include a tab 6394 on the inferior sections 6390. As illustrated in FIG. 48, a superior strap 6305 may be connected to the respective tab 6394. When the four-point arm sleeve 6380 is worn by the patient, the tabs 6394 may be superior to the patient's ears (e.g., at approximately the same position as the tabs 6324 of the conduit headgear 6319). This may allow the superior straps 6305 connected to the tabs 6394 to also be positioned superior to the patient's ears. For example, each tab 6394 may be positioned proximate to a temporal bone, and each superior strap 6305 may overlay the temporal bone and extend toward the occipital bone.

The superior straps 6305 may provide a tensile force directed toward the posterior portion of the patient's head. In the illustrated example, the superior straps 6305 may extend in an inclined direction toward an inferior region of the patient's head (e.g., toward the occipital bone). The tensile force may maintain the four-point arm sleeve 6380 in a desired position (e.g., so that they do not slip over the patient's eyes) and/or provide an additional force to maintain the seal-forming structure 6100 in the sealing position.

In some forms, the positioning and stabilizing structure 6300 provides a force F_{PSS} that assists in maintaining the cushion 6050 in the sealing position on the patient's face. The positioning and stabilizing force F_{PSS} may be the resultant force from the various force vectors of the different elements of the positioning and stabilizing structure 6300.

In the illustrated example in FIG. 48, the patient is oriented in an upright position so force F_{PSS} acts to oppose the external forces acting in that orientation. In other orientations of the patient, the magnitude of the force F_{PSS} (as well as any other force) may change.

For example, the four-point connection sleeve 6380 may provide a sleeve force Fₛₗₑₑᵥₑ in order to hold the seal-forming structure 6100 against the patient's face. As described above, the four-point connection sleeve 6380 may be sized (and/or adjusted) so that it fits snuggly against the patient's head. The sleeve force Fₛₗₑₑᵥₑ may be directed in the superior and/or posterior directions.

The force Fₛₗₑₑᵥₑ may also be directed at least partially in the superior direction in order to overcome the gravitational force F_{g}. The gravitational force F_{g} may be specifically shown for the seal-forming structure 6100 and the plenum chamber 6200, but gravity would act on the entirely of the patient interface 6000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the cushion 6050 in the inferior direction (as viewed in FIG. 48), the frictional force F_{f} would act in the superior direction (e.g., against a patient's face). For example, the patient may experience the frictional force F_{f} against his lip superior (and/or other surfaces of the patient's face in contact with the seal-forming structure 6100) in order to oppose the motion in the inferior direction (which may help to stabilizing the cushion 6050 in place). Although the frictional force F_{f} is shown specifically opposing the gravitational force F_{g} of the cushion 6050, components of an overall frictional force (not shown) would also oppose the gravitational force F_{g} associated with the positioning and stabilizing structure 6300 and any other portions of the patient interface 6000. A force of friction can act along any place where the patient interface 6000 contacts the patient's skin (or hair). The frictional force F_{f} extends in the opposite direction of the gravitational force F_{g} and along the patient's skin (or hair).

Additionally, headgear straps may individually provide a strap force Fₛₜᵣₐₚ in order to hold the seal-forming structure 6100 against the patient's face. Each strap may provide a different strap force Fₛₜᵣₐₚ based on how tight the individual strap was tightened.

In some forms, the sum of the various forces may equal zero so that the patient interface 6000 is at equilibrium (e.g., not moving along the patient's face while in use). Specifically, the gravitational force F_{g} and the blowout force Fₚₗₑₙᵤₘ tend to move the seal-forming structure 6100 away from the desired sealing position. The positioning and stabilizing force F_{PSS} is applied in order to counteract the gravitational force F_{g} and the blowout force Fₚₗₑₙᵤₘ (as well as any frictional forces F_{f}) and keep the seal-forming structure 6100 properly situated. Although the positioning and stabilizing force F_{PSS} may exceed the sum of the other forces and still maintain the seal-forming structure 6100 in an appropriate sealing position, patient comfort may be sacrificed. Maximum patient comfort may be achieved when the net force on the patient interface 6000 is zero and the positioning and stabilizing force F_{PSS} is approximately strong enough to achieve this. As described below, various positions of the patient's head while using the patient interface 6000 may determine the positioning and stabilizing force F_{PSS} necessary to achieve equilibrium.

The magnitude of the forces may change as a result of different in use positions (e.g., when the patient is sleeping in different positions). For example, FIG. 48-1 illustrates the forces acting while the patient sleeps on their back. In this position, the gravitational force F_{g} is directed toward the patient's face and the frictional force F_{f} is directed away from the patient's face. The gravitational force F_{g} may therefore be directed in a similar direction as the force F_{PSS}. Therefore, the gravitational force F_{g} may not oppose the positioning and stabilising force F_{PSS}, which may allow the positioning and stabilising structure 6300 to be less tight and maintain the same sealing force (e.g., an improve patient comfort).

Similarly, FIG. 48-2 illustrates the forces acting while the patient sleeps on their side. In the illustrated example, the gravitational force F_{g} is represented as an “x” within a circle in order to show the force directed into the page. The gravitational force F_{g} may be directed substantially perpendicular to the plenum chamber force Fₚₗₑₙᵤₘ and/or the positioning and stabilizing force F_{PSS}. As described above, the positioning and stabilizing force F_{PSS} may have to counteract both the gravitational force F_{g} and the plenum chamber force Fₚₗₑₙᵤₘ. Also, the plenum chamber 6200 and/or the four-point connection sleeve 6380 may tend to compress on the inferior side, yet be in tension on the superior side. The frictional force F_{f} may still be directed opposite of the gravitational force F_{g} (e.g., out of the page).

In some forms, a tube drag force may provide an additional force on the system. In the tube-down configuration illustrated in FIGS. 48 to 48-2, a tube drag force may act at a location proximate to the patient's nose and/or mouth. Depending on the orientation of the tube (e.g., the angular position of the swivel), the tube drag force may act with the gravitational force F_{g} and/or act with the frictional force F_{f}. This may change throughout use as the position of the tube changes.

### 5.3.10.2.3 Nasal Tube-Up Interface

As illustrated in FIGS. 51 to 54, the patient may wear the cushion 7050 in a tube-up configuration with the conduit headgear 6319 and the two-point headgear 7302. The same conduit headgear 6319 may be used as with the alternate tube-up configuration. In other words, the conduit headgear 6319 may be interchangeable between the tube-up configurations (i.e., the conduit headgear 6319 may connect to either cushion 6050, 7050). As described above, a sleeve also may not be needed with this configuration.

The elements may be assembled as described above. For example, conduits 6320 (via the conduit connection structure 6500), may be used to connect the conduit headgear 6319 to the cushion 7050. The conduit sleeves 6350 are not required with the nasal cushion 7050 because the two-point headgear 7302 is used. However, a sleeve 7350 may be used in order to cover the conduits 6320 and provide a comfortable material against the patient's head.

In other words, the interior of the sleeves 7350 may receive at least a portion of the conduits 6320. A patient cannot directly contact the portion of the conduits 6320 covered by the conduit sleeves 7350. As shown in FIGS. 51 to 52-2, a portion of the conduit headgear 6319 is exposed and contacts the patient while another portion is covered by a sleeve 7350, so that the sleeve 7350 and not the conduit headgear 6319 contacts the patient in that location.

In some forms, because the connection between the conduit headgear 6319 and either cushion is substantially the same, one cushion (e.g., the full-face cushion 6050) may be interchanged for the other cushion (e.g., the nasal cushion 7050). This may be particularly useful in a setting where components are shared among multiple patients. For example, a first patient may use the full-face cushion 6050 and a second patient may use a nasal cushion 7050. The same conduit headgear 6319 may be interchanged between the two cushions 6050, 7050, thus reducing the total number of versions.

As illustrated in FIGS. 51 and 52, the two-point headgear 7302 may connect in two separate locations in order to provide a tensile force that maintains the cushion 7050 in a sealing position on the patient's head.

With continued reference to FIGS. 51 and 52, the conduits 6320 may be positioned along the patient's cheek and may extend to a location superior to the patient's ears. For example, each conduit 6320 may extend along a side of the patient's head toward the crown of the patient's head. This may cause each conduit 6320 to overlay the sphenoid bone and/or the temporal bone, and to extend toward the frontal bone and/or the parietal bone.

The second seal forming structure 6102 may contact the underside of the patient's nose. For example, the seal forming structure 7100 may avoid contact with the patient's nasal ridge and may contact the patient's nose at or below the pronasale and against the columella. The conduits 6320 may provide an additional tensile force that is directed in both the superior and posterior directions (e.g., as viewed in FIG. 52). In this way, the seal forming structure 7100 may be pulled up onto an underside of the patient's nose in a sealing position (e.g., as a result of the superior directed force). The conduits 6320 may also pull the cushion 7050 into the patient's head in order to retain the cushion 6050 in the sealing position (and assist with sealing the first seal forming structure 6101).

In some forms, the conduits 6320 may be generally inextensible and may be structured in order to provide a tensile force when worn by the patient. As described above, the length of the conduits 6320 may be smaller than the patient's head so that they provide tension when worn. Even when the concertina sections 6328 expand, the conduits 6320 may fit snuggly against the patient's head and the passage through the conduits 6320 may remain wide enough to allow a continuous flow of air.

The conduit headgear 6319 may include a tab 6324 on either conduit 6320. As illustrated in FIG. 52, a superior strap 7305 may be connected to the respective tab 6324. When the conduit headgear 6319 is worn by the patient, the tabs 6324 may be superior to the patient's ears. This may allow the superior straps 7305 connected to the tabs 6324 to also be positioned superior to the patient's ears. For example, each tab 6324 may be positioned proximate to a temporal bone, and each superior strap 7305 may overlay the temporal bone and extend toward the occipital bone.

The superior straps 7305 may provide a tensile force directed toward the posterior portion of the patient's head. In the illustrated example, the superior straps 7305 may extend in an inclined direction toward an inferior region of the patient's head (e.g., toward the occipital bone). The tensile force may maintain the conduits 6320 in a desired position (e.g., so that they do not slip over the patient's eyes) and/or provide an additional force to maintain the seal-forming structure 7100 in the sealing position.

In some forms, the presence of the tabs 6324 on the conduits 6320 may allow the different headgear straps to be interchanged with the same conduit headgear 6319. As described above, the superior straps 7305 of the two-point connection headgear 7302 connect to the tabs 6324. The superior straps 6305 of the four-point connection headgear 6302 also connect to the tabs 6324 when used. This common connection point allows for interchangeability between the two styles of headgear 6302, 7302.

In some forms, the positioning and stabilizing structure 6300 provides a force F_{PSS} that assists in maintaining the cushion 7050 in the sealing position on the patient's face. The positioning and stabilizing force F_{PSS} may be the resultant force from the various force vectors of the different elements of the positioning and stabilizing structure 6300.

In the illustrated example in FIG. 52, the patient is oriented in an upright position so force F_{PSS} acts to oppose the external forces acting in that orientation. In other orientations of the patient, the magnitude of the force F_{PSS} (as well as any other force) may change.

For example, the conduit headgear 6319 may provide a tube force F_{tube} in order to hold the seal-forming structure 7100 against the patient's face. As described above, the conduit headgear 6319 may be sized so that it fits snuggly against the patient's head. The concertina sections 6328 may extend in order to provide the necessary F_{tube}. The tube force F_{tube} may be directed in the superior and/or posterior directions.

The force F_{tube} may also be directed at least partially in the superior direction in order to overcome the gravitational force F_{g}. The gravitational force F_{g} may be specifically shown for the seal-forming structure 7100 and the plenum chamber 7200, but gravity would act on the entirely of the patient interface 7000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the cushion 7050 in the inferior direction (as viewed in FIG. 52), the frictional force F_{f} would act in the superior direction (e.g., against a patient's face). For example, the patient may experience the frictional force F_{f} against his lip superior (and/or other surfaces of the patient's face in contact with the seal-forming structure 7100) in order to oppose the motion in the inferior direction (which may help to stabilizing the cushion 7050 in place). Although the frictional force F_{f} is shown specifically opposing the gravitational force F_{g} of the cushion 7050, components of an overall frictional force (not shown) would also oppose the gravitational force F_{g} associated with the positioning and stabilizing structure 7300 and any other portions of the patient interface 7000. A force of friction can act along any place where the patient interface 7000 contacts the patient's skin (or hair). The frictional force F_{f} extends in the opposite direction of the gravitational force F_{g} and along the patient's skin (or hair).

Additionally, headgear straps may individually provide a strap force Fₛₜᵣₐₚ in order to hold the seal-forming structure 7100 against the patient's face. Each strap may provide a different strap force Fₛₜᵣₐₚ based on how tight the individual strap was tightened.

In some forms, the sum of the various forces may equal zero so that the patient interface 6000 is at equilibrium (e.g., not moving along the patient's face while in use). Specifically, the gravitational force F_{g} and the blowout force Fₚₗₑₙᵤₘ tend to move the seal-forming structure 6100 away from the desired sealing position. The positioning and stabilizing force F_{PSS} is applied in order to counteract the gravitational force F_{g} and the blowout force Fₚₗₑₙᵤₘ (as well as any frictional forces F_{f}) and keep the seal-forming structure 6100 properly situated. Although the positioning and stabilizing force F_{PSS} may exceed the sum of the other forces and still maintain the seal-forming structure 6100 in an appropriate sealing position, patient comfort may be sacrificed. Maximum patient comfort may be achieved when the net force on the patient interface 6000 is zero and the positioning and stabilizing force F_{PSS} is exactly strong enough to achieve this. As described below, various positions of the patient's head while using the patient interface 7000 may determine the positioning and stabilizing force F_{PSS} necessary to achieve equilibrium.

The magnitude of the forces may change as a result of different in use positions (e.g., when the patient is sleeping in different positions). For example, FIG. 52-1 illustrates the forces acting while the patient sleeps on their back. In this position, the gravitational force F_{g} is directed toward the patient's face and the frictional force F_{f} is directed away from the patient's face. The gravitational force F_{g} may therefore be directed in a similar direction as the force F_{PSS}. Therefore, the gravitational force F_{g} may not oppose the positioning and stabilising force F_{PSS}, which may allow the positioning and stabilising structure 7300 to be less tight and maintain the same sealing force (e.g., an improve patient comfort).

Similarly, FIG. 52-2 illustrates the forces acting while the patient sleeps on their side. In the illustrated example, the gravitational force F_{g} is represented as an “x” within a circle in order to show the force directed into the page. The gravitational force F_{g} may be directed substantially perpendicular to the plenum chamber force Fₚₗₑₙᵤₘ and/or the positioning and stabilizing force F_{PSS}. As described above, the positioning and stabilizing force F_{PSS} may have to counteract both the gravitational force F_{g} and the plenum chamber force Fₚₗₑₙᵤₘ. Also, the plenum chamber 7200 and/or the conduit headgear 6319 may tend to compress on the inferior side, yet be in tension on the superior side. The frictional force F_{f} may still be directed opposite of the gravitational force F_{g} (e.g., out of the page).

In some forms, a tube drag force may provide an additional force on the system. In the tube-up configuration illustrated in FIGS. 52 to 52-2, a tube drag force may act at a superior region of the patient's head. Depending on the orientation of the tube (e.g., the angular position of the swivel), the tube drag force may assist the positioning and stabilising force F_{PSS} and/or oppose the positioning and stabilising force F_{PSS}. This may change throughout use as the position of the tube changes.

### 5.3.10.2.4 Nasal Tube-Down Interface

As illustrated in FIGS. 55 to 59, the patient may wear the cushion 7050 in a tube-down configuration with the rigidizer arms 6340, the two-point headgear 7302, and the two-point arm sleeve 6380-1. The same cushion 7050 and the two-point headgear 7302 may be used in both nasal configurations (e.g., FIGS. 51-54 and 55-58).

The elements may be assembled as described above. For example, the two-point arm sleeve 6380-1 may be connected to the rigidizer arms 6340. The rigidizer arms 6340 (via the arm connection structure 6504), may be used to connect the rigidizer arms 6340 to the cushion 7050.

As illustrated in FIGS. 55 and 56, the two-point headgear 7302 may connect in two separate locations in order to provide a tensile force that maintains the cushion 7050 in a sealing position on the patient's head.

With continued reference to FIGS. 55 and 56, the rigidizer arms 6340 (covered by the two-point arm sleeve 6380-1) may be positioned along the patient's cheek and may extend to a location superior to the patient's ears. For example, each rigidizer arm 6340 may extend along a side of the patient's head toward the crown of the patient's head. This may cause each rigidizer arm 6340 to overlay the sphenoid bone and/or the temporal bone, and to extend toward the frontal bone and/or the parietal bone. In some forms, the rigidizer arm 6340 may extend along a similar path of the patient's face as the conduit 6320.

In these figures, the rigidizer arms 6340 may be covered by the two-point arm sleeve 6380-1 so that each rigidizer arm 6340 is positioned within the interior of the two-point arm sleeve 6380-1. The rigidizer arms 6340 are therefore not visible and do not directly contact the patient

The seal forming structure 7100 may contact the underside of the patient's nose. For example, the seal forming structure 7100 may avoid contact with the patient's nasal ridge and may contact the patient's nose at or below the pronasale and against the columella. The two-point arm sleeve 6380-1 and the rigidizer arms 6340 may provide an additional tensile force that is directed in both the superior and posterior directions (e.g., as viewed in FIG. 56). In this way, the seal forming structure 7100 may be pulled up onto an underside of the patient's nose in a sealing position (e.g., as a result of the superior directed force). The two-point arm sleeve 6380-1 and/or the rigidizer arms 6340 may also pull the cushion 7050 into the patient's head in order to retain the cushion 7050 in the sealing position.

In some forms, the rigidizer arms 6340 may be generally inextensible and may be structured in order to provide a tensile force when worn by the patient. The two-point arm sleeve 6380-1 may also be at least partially inextensible along its length (although it may be able to stretch). The superior section 6392-1 of the two-point arm sleeve 6380-1 may be adjustable (e.g., via hook and loop material) in order tighten the two-point arm sleeve 6380-1 against the patient's head and create the tensile force.

The two-point arm sleeve 6380-1 may include a tab 6394-1 on the inferior sections 6390-1. As illustrated in FIG. 56, a superior strap 7305 may be connected to the respective tab 6394-1. When the two-point arm sleeve 6380-1 is worn by the patient, the tabs 6394-1 may be superior to the patient's ears (e.g., at approximately the same position as the tabs 6324 of the conduit headgear 6319). This may allow the superior straps 6305 connected to the tabs 6394-1 to also be positioned superior to the patient's ears. For example, each tab 6394-1 may be positioned proximate to a temporal bone, and each superior strap 6305 may overlay the temporal bone and extend toward the occipital bone.

The superior straps 6305 may provide a tensile force directed toward the posterior portion of the patient's head. In the illustrated example, the superior straps 6305 may extend in an inclined direction toward an inferior region of the patient's head (e.g., toward the occipital bone). The tensile force may maintain the four-point arm sleeve 6380 in a desired position (e.g., so that they do not slip over the patient's eyes) and/or provide an additional force to maintain the seal-forming structure 7100 in the sealing position.

In some forms, the tabs 6394, 6394-1 on the respective sleeves 6380, 6380-1 may be positioned in approximately the same location on a patient's head. As described above, the superior straps 7305 of the two-point connection headgear 7302 connect to the tabs 6394-1. The superior straps 6305 of the four-point connection headgear 6302 also connect to the tabs 6394 when used. The two styles of sleeves 6380, 6380-1 with similarly located tabs 6394, 6394-1, facilitate the interchangeability of the four-point and two point connection headgear 6302, 7302 with the same rigidizer arms 6340.

In some forms, the positioning and stabilizing structure 6300 provides a force F_{PSS} that assists in maintaining the cushion 6050 in the sealing position on the patient's face. The positioning and stabilizing force F_{PSS} may be the resultant force from the various force vectors of the different elements of the positioning and stabilizing structure 6300.

In the illustrated example in FIG. 56, the patient is oriented in an upright position so force F_{PSS} acts to oppose the external forces acting in that orientation. In other orientations of the patient, the magnitude of the force F_{PSS} (as well as any other force) may change.

For example, the two-point connection sleeve 6380-1 may provide a sleeve force Fₛₗₑₑᵥₑ in order to hold the seal-forming structure 7100 against the patient's face. As described above, the two-point connection sleeve 6380-1 may be sized (and/or adjusted) so that it fits snuggly against the patient's head. The sleeve force Fₛₗₑₑᵥₑ may be directed in the superior and/or posterior directions.

The force Fₛₗₑₑᵥₑ may also be directed at least partially in the superior direction in order to overcome the gravitational force F_{g}. The gravitational force F_{g} may be specifically shown for the seal-forming structure 7100 and the plenum chamber 7200, but gravity would act on the entirely of the patient interface 7000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the cushion 7050 in the inferior direction (as viewed in FIG. 56), the frictional force F_{f} would act in the superior direction (e.g., against a patient's face). For example, the patient may experience the frictional force F_{f} against his lip superior (and/or other surfaces of the patient's face in contact with the seal-forming structure 7100) in order to oppose the motion in the inferior direction (which may help to stabilizing the cushion 7050 in place). Although the frictional force F_{f} is shown specifically opposing the gravitational force F_{g} of the cushion 7050, components of an overall frictional force (not shown) would also oppose the gravitational force F_{g} associated with the positioning and stabilizing structure 7300 and any other portions of the patient interface 7000. A force of friction can act along any place where the patient interface 7000 contacts the patient's skin (or hair). The frictional force F_{f} extends in the opposite direction of the gravitational force F_{g} and along the patient's skin (or hair).

Additionally, headgear straps may individually provide a strap force Fₛₜᵣₐₚ in order to hold the seal-forming structure 7100 against the patient's face. Each strap may provide a different strap force Fₛₜᵣₐₚ based on how tight the individual strap was tightened.

In some forms, the sum of the various forces may equal zero so that the patient interface 7000 is at equilibrium (e.g., not moving along the patient's face while in use). Specifically, the gravitational force F_{g} and the blowout force Fₚₗₑₙᵤₘ tend to move the seal-forming structure 7100 away from the desired sealing position. The positioning and stabilizing force F_{PSS} is applied in order to counteract the gravitational force F_{g} and the blowout force Fₚₗₑₙᵤₘ (as well as any frictional forces F_{f}) and keep the seal-forming structure 7100 properly situated. Although the positioning and stabilizing force F_{PSS} may exceed the sum of the other forces and still maintain the seal-forming structure 7100 in an appropriate sealing position, patient comfort may be sacrificed. Maximum patient comfort may be achieved when the net force on the patient interface 7000 is zero and the positioning and stabilizing force F_{PSS} is exactly strong enough to achieve this. As described below, various positions of the patient's head while using the patient interface 7000 may determine the positioning and stabilizing force F_{PSS} necessary to achieve equilibrium.

The magnitude of the forces may change as a result of different in use positions (e.g., when the patient is sleeping in different positions). For example, FIG. 56-1 illustrates the forces acting while the patient sleeps on their back. In this position, the gravitational force F_{g} is directed toward the patient's face and the frictional force F_{f} is directed away from the patient's face. The gravitational force F_{g} may therefore be directed in a similar direction as the force F_{PSS}. Therefore, the gravitational force F_{g} may not oppose the positioning and stabilising force F_{PSS}, which may allow the positioning and stabilising structure 7300 to less tight and maintain the same sealing force (e.g., an improve patient comfort).

Similarly, FIG. 56-2 illustrates the forces acting while the patient sleeps on their side. In the illustrated example, the gravitational force F_{g} is represented as an “x” within a circle in order to show the force directed into the page. The gravitational force F_{g} may be directed substantially perpendicular to the plenum chamber force Fₚₗₑₙᵤₘ and/or the positioning and stabilizing force F_{PSS}. As described above, the positioning and stabilizing force F_{PSS} may have to counteract both the gravitational force F_{g} and the plenum chamber force Fₚₗₑₙᵤₘ. Also, the plenum chamber 7200 and/or the two-point connection sleeve 6380-1 may tend to compress on the inferior side, yet be in tension on the superior side. The frictional force F_{f} may still be directed opposite of the gravitational force F_{g} (e.g., out of the page).

In some forms, a tube drag force may provide an additional force on the system. In the tube-down configuration illustrated in FIGS. 48 to 48-2, a tube drag force may act at a location proximate to the patient's nose and/or mouth. Depending on the orientation of the tube (e.g., the angular position of the swivel), the tube drag force may act with the gravitational force F_{g} and/or act with the frictional force F_{f}. This may change throughout use as the position of the tube changes.

### 5.3.10.2.5 Modularity of Elements

FIG. 59 illustrates how the different elements can be combined in order to form the four different patient interfaces described above. As illustrated, the different components may be reused for different styles of patient interfaces. This may allow for easier manufacturing and assembly, because a large number of the same components may be produced and used in a variety of styles. The only components not used in multiple styles may be the sleeves. However, the sleeves may be easier to manufacture.

### 5.4 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, at least 6 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller, a therapy device controller, a pressure generator 4140, one or more protection circuits, memory, transducers 4270, data communication interface and one or more output devices. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000.

### 5.4.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 5.4.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. No. 7,866,944; U.S. Patent No. 8,638,14; U.S. Pat. No. 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000.

In one form, a signal from a transducer 4270 may be filtered, such as by low-pass, high-pass or band-pass filtering.

### 5.4.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.4.2 RPT Device Algorithms

As mentioned above, in some forms of the present technology, the central controller may be configured to implement one or more algorithms expressed as computer programs stored in a non-transitory computer readable storage medium, such as memory. The algorithms are generally grouped into groups referred to as modules.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000.

As shown in FIG. 42, the air circuit 4170 may be a hose that can convey the flow of pressurized air. The air circuit 4170 may be connected directly to the plenum chamber 6200, 7200 (e.g., in the respective vent opening 6402, 7402). In this configuration (i.e., “tube down”), the patient interface 6000, 7000 may not include a vent 6400. Instead the hose of the air circuit 4170 may include a vent.

The air circuit 4170 may also be used in the tube up configuration and may be connected to the inlet 6332.

### 5.6 Humidifier

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.6.2 Humidifier Components

### 5.6.2.1 Water Reservoir

According to one arrangement, the humidifier 5000 may comprise a water reservoir 5110 configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building's water supply system.

According to one aspect, the water reservoir 5110 is configured to add humidity to a flow of air from the RPT device 4000 as the flow of air travels therethrough. In one form, the water reservoir 5110 may be configured to encourage the flow of air to travel in a tortuous path through the reservoir 5110 while in contact with the volume of water therein.

According to one form, the reservoir 5110 may be removable from the humidifier 5000, for example in a lateral direction as shown in FIG. 5A and FIG. 5B.

The reservoir 5110 may also be configured to discourage egress of liquid therefrom, such as when the reservoir 5110 is displaced and/or rotated from its normal, working orientation, such as through any apertures and/or in between its sub-components. As the flow of air to be humidified by the humidifier 5000 is typically pressurised, the reservoir 5110 may also be configured to prevent losses in pneumatic pressure through leak and/or flow impedance.

### 5.6.2.2 Conductive Portion

According to one arrangement, the reservoir 5110 comprises a conductive portion 5120 configured to allow efficient transfer of heat from the heating element 5240 to the volume of liquid in the reservoir 5110. In one form, the conductive portion 5120 may be arranged as a plate, although other shapes may also be suitable. All or a part of the conductive portion 5120 may be made of a thermally conductive material such as aluminium (e.g. approximately 2 mm thick, such as 1 mm, 1.5 mm, 2.5 mm or 3 mm), another heat conducting metal or some plastics. In some cases, suitable heat conductivity may be achieved with less conductive materials of suitable geometry.

### 5.6.2.3 Humidifier Reservoir Dock

In one form, the humidifier 5000 may comprise a humidifier reservoir dock 5130 (as shown in FIG. 5B) configured to receive the humidifier reservoir 5110. In some arrangements, the humidifier reservoir dock 5130 may comprise a locking feature such as a locking lever 5135 configured to retain the reservoir 5110 in the humidifier reservoir dock 5130.

### 5.6.2.4 Water Level Indicator

The humidifier reservoir 5110 may comprise a water level indicator 5150 as shown in FIG. 5A-5B. In some forms, the water level indicator 5150 may provide one or more indications to a user such as the patient 1000 or a care giver regarding a quantity of the volume of water in the humidifier reservoir 5110. The one or more indications provided by the water level indicator 5150 may include an indication of a maximum, predetermined volume of water, any portions thereof, such as 25%, 50% or 75% or volumes such as 200 ml, 300 ml or 400 ml.

### 5.6.2.5 Heating Element

A heating element 5240 may be provided to the humidifier 5000 in some cases to provide a heat input to one or more of the volume of water in the humidifier reservoir 5110 and/or to the flow of air. The heating element 5240 may comprise a heat generating component such as an electrically resistive heating track. One suitable example of a heating element 5240 is a layered heating element such as one described in the PCT Patent Application Publication No. WO 2012/171072, which is incorporated herewith by reference in its entirety.

In some forms, the heating element 5240 may be provided in the humidifier base 5006 where heat may be provided to the humidifier reservoir 5110 primarily by conduction as shown in FIG. 5B.

### 5.7 Breathing Waveforms

FIG. 6 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Glossary

For the purposes of the present technology disclosure, in certain forms of the present technology, one or more of the following definitions may apply. In other forms of the present technology, alternative definitions may apply.

### 5.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar 0.001 atm). In this specification, unless otherwise stated, pressure is given in units of cmH2O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

5.8.1.1 Materials & their Properties

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45.

Polycarbonate: a thermoplastic polymer of Bisphenol-A Carbonate.

### 5.8.1.2 Mechanics

Deformation: The process where the original geometry of a member changes when subjected to forces, e.g. a force in a direction with respect to an axis. The process may include stretching or compressing, bending and, twisting.

Stiffness: The ability of a structure or component to resist deformation in response to an applied load. A structure or component may have an axial stiffness, a bending stiffness, and a torsional stiffness. A structure or component is said to be stiff when it does not deform easily when subject to mechanical forces. Stiffness of a structure or component is related to its material properties and its shape. The inverse of stiffness is flexibility.

Elasticity: The ability of a material to return to its original geometry after deformation.

Viscous: The ability of a material to resist flow.

Visco-elasticity: The ability of a material to display both elastic and viscous behaviour in deformation.

Yield: The situation when a material can no longer return back to its original geometry after deformation.

### 5.8.1.3 Structural Elements

Beam: A beam will be taken to mean an element that is relatively long in one direction.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Membrane: Membrane will be taken to mean a typically thin element that has, preferably, substantially no resistance to bending, but has resistance to being stretched. There may also be resistance to compression. The membrane may be relatively long in two dimensions with one thin dimension.

Plate: A plate will be taken to mean relatively long in two dimensions with one thin dimension. The plate has bending, tensile, and compressive stiffness.

Load transfer member: A structural member which transfers load from one location to another.

Load support member: A structural member which transfers load from one location to a non-structural item, such as the face.

Tension member: A structural element that is subjected to tensional forces.

Tie (noun): A structure designed to resist tension.

Compression member: A structural element that is subjected to compression forces.

Strut: A strut will be taken to be a structural component designed to increase the compression resistance of another component in at least one direction.

Stiffener: A stiffener will be taken to mean a structural component designed to increase the bending resistance of another component in at least one direction.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.8.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.8.3 Anatomy

### 5.8.3.1 Anatomy of the Face

Ala: the external outer wall or “wing” of each nostril (plural: alar).

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion.

### 5.8.3.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.8.3.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.8.4 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilizing structure designed to hold a device, e.g. a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.8.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.8.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.8.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical—topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.8.5.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 5.8.5.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term as used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

5.10 REFERENCE SIGNS LIST

| | |
|---|---|
| patient | 1000 |
| bed partner | 1100 |
| patient interface | 3000 |
| seal-forming structure | 3100 |
| plenum chamber | 3200 |
| chord | 3210 |
| superior point | 3220 |
| inferior point | 3230 |
| positioning and stabilizing structure | 3300 |
| vent | 3400 |
| connection port | 3600 |
| forehead support | 3700 |
| RPT device | 4000 |
| external housing | 4010 |
| upper portion | 4012 |
| portion | 4014 |
| panel | 4015 |
| chassis | 4016 |
| handle | 4018 |
| pneumatic block | 4020 |
| air filter | 4110 |
| inlet air filter | 4112 |
| outlet air filter | 4114 |
| muffler | 4120 |
| inlet muffler | 4122 |
| outlet muffler | 4124 |
| pressure generator | 4140 |
| blower | 4142 |
| motor | 4144 |
| anti - spill back valve | 4160 |
| air circuit | 4170 |
| air circuit | 4171 |
| electrical components | 4200 |
| single Printed Circuit Board Assembly | 4202 |
| power supply | 4210 |
| input device | 4220 |
| transducer | 4270 |
| humidifier | 5000 |
| humidifier inlet | 5002 |
| humidifier outlet | 5004 |
| humidifier base | 5006 |
| humidifier reservoir | 5110 |
| conductive portion | 5120 |
| humidifier reservoir dock | 5130 |
| locking lever | 5135 |
| water level indicator | 5150 |

1. A patient interface comprising:
a plenum chamber pressurisable to a therapeutic pressure of at least 4 cmH₂O above ambient air pressure, said plenum chamber including at least one plenum chamber inlet port sized and structured to receive a flow of air at the therapeutic pressure for breathing by a patient, a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a positioning and stabilising structure to provide a force to hold the seal-forming structure in a therapeutically effective position on the patient's head, the positioning and stabilising structure including:
an inextensible element configured to overlay the patient's cheeks,
a sleeve configured to cover at least a portion of the inextensible element, the sleeve being removably positioned around the at least a portion of the inextensible element, and
headgear straps configured to provide at least a portion of the force to maintain the seal-forming structure in a sealing position;
wherein
the patient interface is configured to allow the patient to breathe from ambient through their mouth in the absence of a flow of pressurised air through the plenum chamber inlet port, or the patient interface is configured to leave the patient's mouth uncovered.

2. The patient interface of claim 1, wherein:
the inextensible element is a conduit headgear including a pair of conduits each configured to convey the flow of air at the therapeutic pressure to the plenum chamber;
the sleeve is a first conduit sleeve, the positioning and stabilising structure further includes a second conduit sleeve having the same structure as the first conduit sleeve, the second conduit sleeve being removably connected to one conduit of the pair of conduits, wherein the first and second conduit sleeves each include:
a longitudinal extension forming a pathway that extends between a superior opening and an inferior opening, the pathway configured to receive one conduit of the pair of conduits;
an inferior extension positioned outside of the pathway and proximate to the inferior opening; and
a connection member connected to the inferior extension.

3. The patient interface of claim 1, wherein:
the inextensible element is a pair of rigidizer arms configured to extend along a contour of the patient's face;
the sleeve is a single sleeve configured to receive both rigidizer arms of the pair of rigidizer arms, the sleeve includes:
a superior section; and
a pair of inferior sections, wherein each inferior section of the pair of inferior sections is connected to the superior section, wherein each inferior section of the pair of inferior sections includes a longitudinal extension that forms a pathway with an inferior opening, the pathway configured to receive one rigidizer arm of the pair of rigidizer arms, wherein each inferior section of the pair of inferior sections further includes:
each inferior section of the pair of inferior sections includes an inferior extension connected proximate to the inferior opening, the inferior extension being positioned outside of the pathway, and
a connection member connected to the inferior extension.

4. The patient interface of claim 1, wherein:
the inextensible element is a pair of rigidizer arms configured to extend along a contour of the patient's face;
the sleeve is a single sleeve configured to receive both rigidizer arms of the pair of rigidizer arms, the sleeve includes:
a superior section that is substantially inextensible; and
a pair of inferior sections that are at least partially extensible, wherein each inferior section of the pair of inferior sections is connected to the superior section, wherein each inferior section of the pair of inferior sections includes a longitudinal extension that forms a pathway with an inferior opening, the pathway configured to receive one rigidizer arm of the pair of rigidizer arms, wherein each pathway is isolated from the other pathway.

5. The patient interface of claim 1, wherein the inextensible element is a conduit headgear including a pair of conduits each configured to convey the flow of air at the therapeutic pressure to the plenum chamber.

6. The patient interface of claim 5, wherein each conduit of the pair of conduits includes a tab, and wherein superior straps of the headgear straps configured to removably connect to the tabs.

7. The patient interface of claim 6, wherein each tab is configured to be positioned superior to the patient's ears, in use.

8. The patient interface of claim 5, wherein the at least one plenum chamber inlet is a pair of plenum chamber inlet ports, the pair of conduits removably connected to the pair of plenum chamber inlet ports.

9. The patient interface of claim 8, wherein each conduit of the pair of conduits includes a clip configured to engage one plenum chamber inlet port of the pair of plenum chamber inlet ports.

10. The patient interface of claim 5, wherein the sleeve includes a longitudinal extension forming a pathway that extends between a superior opening and an inferior opening, the pathway configured to receive one conduit of the pair of conduits.

11. The patient interface of claim 10, wherein material around the superior opening and/or the inferior opening is elastic and configured to allow the superior opening and/or the inferior opening to stretch and expand a width of the respective opening.

12. The patient interface of claim 10, wherein the material between the superior opening and the inferior opening is substantially inextensible.

13. The patient interface of claim 10, wherein the inferior opening is configured to be positioned proximate to the plenum chamber, in use.

14. The patient interface of claim 10, wherein the superior opening is configured to be positioned at a location inferior to the patient's ear, in use.

15. The patient interface of claim 10, wherein the sleeve includes an inferior extension positioned outside of the pathway and proximate to the inferior opening.

16. The patient interface of claim 15, wherein the inferior extension is more rigid than the pathway.

17. The patient interface of claim 15, wherein the inferior extension is formed with a rigid material.

18. The patient interface of claim 15, wherein the inferior extension is rigidized using a stitching method.

19. The patient interface of claim 15, wherein the inferior extension is substantially inextensible.

20. The patient interface of claim 15, further comprising a connection member connected to the inferior extension.

21. The patient interface of claim 20, wherein the connection member is a magnet.

22. The patient interface of claim 20, wherein the inferior opening and the connection member are oriented in opposite directions.

23. The patient interface of claim 5, wherein the sleeve is a first conduit sleeve, the positioning and stabilising structure further includes a second conduit sleeve having the same structure as the first conduit sleeve, the second conduit sleeve being removably connected to one conduit of the pair of conduits.

24. The patient interface of claim 1, wherein the seal-forming structure is configured to form a seal around the patient's mouth and the patient's nares.

25. The patient interface of claim 1, wherein the inextensible element is a pair of rigidizer arms configured to extend along a contour of the patient's face.

26. The patient interface of claim 25, wherein the plenum chamber inlet port is configured to align with the patient's mouth, the plenum chamber further including a pair of arm openings, and wherein the pair of rigidizer arms being removably connected to the pair of arm openings.

27. The patient interface of claim 25, wherein the pair of rigidizer arms are flexible in one direction and rigid in another direction, the pair of rigidizer arms are configured to bend in order to conform to the shape of the patient's cheeks.

28. The patient interface of claim 25, wherein each rigidizer arm of the pair of rigidizer arms includes a free end and a clip opposite to the free end, the clip configured to engage one arm opening of the pair of arm openings.

29. The patient interface of claim 28, wherein each clip is configured to limit airflow through the respective arm opening.

30. The patient interface of claim 25, wherein the sleeve is a single sleeve configured to receive both rigidizer arms of the pair of rigidizer arms.

31. The patient interface of claim 25, wherein the sleeve includes:
a superior section;
a pair of inferior sections, wherein each inferior section of the pair of inferior sections is connected to the superior section, wherein each inferior section of the pair of inferior sections includes a longitudinal extension that forms a pathway with an inferior opening, the pathway configured to receive one rigidizer arm of the pair of rigidizer arms.

32. The patient interface of claim 31, wherein each pathway is isolated from the other pathway.

33. The patient interface of claim 31, wherein the superior section is constructed from a different material than the pair of inferior sections.

34. The patient interface of claim 33, wherein the superior section is substantially inextensible and the pair of inferior sections are at least partially extensible.

35. The patient interface of claim 34, wherein material around the inferior opening is elastic and configured to allow the inferior opening to stretch and expand a width of the respective opening.

36. The patient interface of claim 31, wherein the superior section includes a length-adjustable section and is configured to be adjusted based on a size of the patient's head.

37. The patient interface of claim 31, wherein each inferior section of the pair of inferior sections further includes a tab disposed proximate to the superior section and configured to receive a headgear strap.

38. The patient interface of claim 31, wherein each inferior section of the pair of inferior sections includes an inferior extension connected proximate to the inferior opening, the inferior extension being positioned outside of the pathway.

39. The patient interface of claim 38, wherein the inferior extension is more rigid than the pathway.

40. The patient interface of claim 38, wherein the inferior extension is form with a rigid material.

41. The patient interface of claim 38, wherein the inferior extension is rigidized using a stitching method.

42. The patient interface of claim 38, wherein the inferior extension is substantially inextensible.

43. The patient interface of claim 38, further comprising a connection member connected to the inferior extension.

44. The patient interface of claim 43, wherein the connection member is a magnet.

45. The patient interface of claim 43, wherein the connection member and the connection member are oriented in opposite directions.

46. The patient interface of claim 25, wherein the seal-forming structure is configured to form a seal around the patient's mouth and the patient's nares.

47. The patient interface of claim 25, wherein the seal-forming structure is configured to form a seal around the patient's nares and is configured to leave the patient's mouth exposed to ambient.

48.-81. (canceled)
